(19) United States
(12) Patent Application Publication
(10) Pub. No.: US 2006/0264493 A1
(43) Pub. Date: Nov. 23, 2006

(54) Tetracyclic pyrazole derivatives as kinase inhibitors, process for their preparation and pharmaceutical compositions comprising them

(76) Inventors: Ermes Vanotti, Milan (IT); Giovanni Cervi, Como (IT); Maurizio Pulici, Milan (IT); Maria Menichincheri, Milan (IT); Mario Varasi, Milan (IT); Paola Vianello, Milan (IT)

Correspondence Address: SCULLY, SCOTT, MURPHY & PRESSER, 400 GARDEN CITY PLAZA, SUITE 300, GARDEN CITY, NY, 11530, US

(21) Appl. No.: 10/545,768

(22) PCT Filed: Feb. 3, 2004
(86) PCT No.: PCT/EP04/50071

§ 371 Date: Jun. 22, 2006

Related U.S. Application Data
(60) Provisional application No. 60/448,049, filed on Feb. 17, 2003.

(51) Int. Cl. A61K 31/4162; C07D 487/02

(52) U.S. Cl. 514/406; 548/358.5

(57) ABSTRACT

The present invention provides a method for treating diseases caused by and/or associated with an altered protein kinase activity which comprises administering to a mammal in need thereof an effective amount of a tetracyclic pyrazole. The invention also provides specific tetracyclic pyrazole derivatives, useful intermediates, a library comprising at least two of them, a process for their preparation and the pharmaceutical compositions containing them, which are useful in the treatment of diseases caused by and/or associated with an altered protein kinase activity such as cancer, cell proliferative disorders, viral infections, autoimmune diseases and neurodegenerative disorders.

### BACKGROUND OF THE INVENTION

1. Field of the Invention

The present invention relates to pyrazole derivatives active as kinase inhibitors and, more in particular, it relates to tetracyclic pyrazole derivatives, to a process for their preparation, to pharmaceutical compositions comprising them and to their use as therapeutic agents, particularly in the treatment of diseases linked to deregulated protein kinases.

2. Discussion of the Background

The malfunctioning of protein kinases (PKs) is the hallmark of numerous diseases.

A large share of the oncogenes and proto-oncogenes are involved in human cancers code for PKs. The enhanced activities of PKs are also implicated in many non-malignant diseases such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

Several pyrazole derivatives and analogues thereof are known in the art, for instance as synthetic intermediates or even as therapeutic agents.

As an example, carboxamido-pyrazoles possessing cdk inhibitory activity have been described in U.S. Pat. No. 6,218,418 to Pevarello et al.

Pyrazole derivatives have been described for use in the treatment of inflammation. U.S. Pat. No. 5,134,142 to Matsuo et al describes 1,5-diaryl pyrazole derivatives, and specifically, 1-(4-fluorophenyl)-5-[4-(methylsulfonyl)phenyl]-3-trifluoromethyl pyrazole, as having anti-inflammatory activity.

U.S. Pat. No. 3,940,418 to R. Hamilton describes tricyclic 4,5-dihydrobenz[g]indazole derivatives as anti-inflammatory agents. In addition, R. Hamilton [J. Heterocyclic Chem., 13, 545 (1976)] describes tricyclic 4,5-dihydrobenz[g]indazole derivatives as anti-inflammatory agents. U.S. Pat. No. 5,134,155 describes fused tricyclic pyrazole derivatives having a saturated ring bridging the pyrazole and a phenyl radical as HMG-CoA reductase inhibitors. European publication EP 477,049, published Mar. 25, 1992, describes [4,5-dihydro-1-phenyl-1H-benz[g]indazol-3-yl]amides as having antipsychotic activity. European publication EP 347,773, published Dec. 27, 1989, describes (4,5-dihydro-1-phenyl-1H-benz[g]indazol-3-yl]propanamide derivatives as immunostimulants. M. Hashem et al [J. Med. Chem., 19, 229 (1976)] describes fused tricyclic pyrazole derivatives, having a saturated ring bridging the pyrazole and a phenyl radical, as antibiotics.

Certain substituted pyrazolyl-benzenesulfonamide derivatives have been described in the literature as synthetic intermediates. Specifically, 4-[5-(4-chlorophenyl)-3-phenyl-1H-pyrazol-1-yl]benzenesulfonamide has been prepared from a pyrazoline compound as an intermediate for compounds having hypoglycemic activity [R. Soliman et al, J. Pharm. Sci., 76, 626 (1987)]. 4-[5-[2-(4-Bromophenyl)-2H-1,2,3-triazol-4-yl]-3-methyl-1H-pyrazol-1-yl]benzenesulfonamide has been prepared from a pyrazoline compound and described as potentially having hypoglycemic activity [H. Mokhtar, Pak. J. Sci. Ind. Res., 31, 762 (1988)]. Similarly, 4-[4-bromo-5-[2-(4-chlorophenyl)-2H-1,2,3-triazol-4-yl]-3-methyl-1H-pyrazol-1-yl]benzenesulfonamide has been prepared [H. Mokhtar et al, Pak. J. Sci. Ind. Res., 34, 9 (1991)].

The phytotoxicity of pyrazole derivatives is described (M. Cocco et al, Il. Farmaco-Ed. Sci., 40, 272 (1985)], specifically for 1-[4-(aminosulfonyl)phenyl]-5-phenyl-1H-pyrazole-3,4-dicarboxylic acid.

The use of styryl pyrazole esters for antidiabetic drugs is described (H. Mokhtar et al, Pharmazie, 33, 649-651 (1978)]. The use of styryl pyrazole carboxylic acids for antidiabetes drugs is described [R. Soliman et al, Pharmazie, 33, 184-5 (1978)]. The use of 4-[3,4,5-trisubstituted-pyrazol-1-yl]benzenesulfonamide derivatives as intermediates for sulfonylurea anti-diabetes agents is described, and specifically, 1-[4-(aminosulfonyl)phenyl]-3-methyl-5-phenyl-1H-pyrazole-4-carboxylic acid [R. Soliman et al, J. Pharm. Sci., 72, 1004 (1983)]. A series of 4-[3-substituted methyl-5-phenyl-1H-pyrazol-1-yl]benzenesulfonamide derivatives has been prepared as intermediates for anti-diabetes agents, and more specifically, 4-[3-methyl-5-phenyl-1H-pyrazol-1-yl]benzenesulfonamide [H. Feid-Allah, Pharmazie, 36, 754 (1981)]. In addition, 1-(4-[aminosulfonyl]phenyl)-5-phenylpyrazole-3-carboxylic acid has been prepared from the above described 4-[3-methyl-5-phenyl-1H-pyrazol-1-yl]benzenesulfonamide compound [R. Soliman et al, J. Pharm. Sci., 70, 602 (1981)].

WO00/27822 discloses tricyclic pyrazole derivatives, WO00/59901 discloses dihydroindeno pyrazole derivatives, WO95/15315 discloses diphenyl pyrazole compounds, WO95/15317 discloses triphenyl pyrazole compounds, WO95/15318 discloses tri-substituted pyrazole compounds, and WO96/09293 discloses benz[g]indazolyl derivatives. WO95/15316 discloses substituted pyrazolyl benzenesulfamide derivatives.

### SUMMARY OF THE INVENTION

The present inventors have now discovered that some tetracyclic pyrazole derivatives are endowed with multiple protein kinase inhibiting activity and are thus useful in therapy in the treatment of diseases caused by and/or associated with disregulated protein kinases.

As such, it is an object of the invention to provide compounds useful as therapeutic agents against a host of diseases caused by a disregulated protein kinase activity.

It is another object to provide compounds endowed with multiple protein kinase inhibiting activity.

More specifically, the tetracyclic, pyrazole derivatives of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xenoderoma pigmentosum, keratoctanthoma, thyroid follicular cancer and Kaposi's sarcoma.

Due to the key role of PKs in the regulation of cellular proliferation, these tetracyclic pyrazole derivatives are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

The compounds of the invention can be useful in the treatment of Alzheimer's disease, as suggested by the fact that cdk5 is involved in the phosphorylation of tau protein (J. Biochem., 1995, 117, 741-749).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis.

The compounds of this invention may also act as inhibitors of other protein kinases, e.g. protein kinase C in different isoforms, Met, PAK-4, PAK-5, ZC-1, STLK-2, DDR-2, Aurora 1, Aurora 2, Bub-1, PLK, Chk1, Chk2, HER2, raf1, MEK1, MAPK, EGF-R, PDGF-R, FGF-R, IGF-R, PI3K, weel kinase, Src, Abl, Akt, ILK, MK-2, IKK-2, Nek, Cdc7, and thus be effective in the treatment of diseases associated with other protein kinase malfunctioning.

Accordingly, the present invention provides a method for treating diseases caused by and/or associated with an altered protein kinase activity which comprises administering to a mammal in need thereof an effective amount of a tetracyclic pyrazole derivative represented by formula (I):

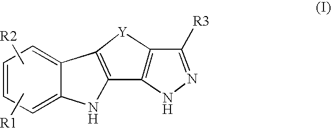

wherein

R1 and R2, being the same or different, are independently hydrogen or halogen atom, nitro, cyano, hydroxy, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl arylaminocarbonyl, heteroarylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl, aryl C₁-C₆ alkyl group, aryl C₁-C₆ alkyloxy group, aryloxy, heteroaryl, heteroaryl C₁-C₆ alkyl group, a straight or branched C₁-C₆ alkylthio, C₁-C₆ alkylsulphinyl, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆alkyloxycarbonylamino, aryloxycarbonylamino, ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆alkylaminosulfonyl and arylaminosulfonyl group;

Y is a —(CH₂)ₙ— group wherein n is 1, 2 or 3, or a carbon-carbon double bond (—CH₂═CH₂—);

R3 is hydrogen atom, cyano, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, an aryl, an aryl C₁-C₆ alkyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxy-carbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkyloxyaminocarbonyl, aryloxyamino carbonyl, C₁-C₆ alkylcarbonyloxy, arylcarbonyloxy,

C₁-C₆ alkylcarbonyl, arylcarbonyl, heterocyclylcarbonyl, aryl C₁-C₆ alkyloxy group, aryloxy, a straight or branched C₁-C₆ alkylthio, aryl C₁-C₆ alkylthio, C₁-C₆ alkylsulphinyl group, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, an ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonyl-amino, arylaminothiocarbonylamino, C₁-C₆ alkylsulfonylamino, arylsulfonylamino, C₁-C₆ alkylaminosulfonyl, and arylamino-sulfonyl group, or a pharmaceutically acceptable salt thereof.

The present invention also provides a tetracyclic pyrazole derivative of the formula (I):

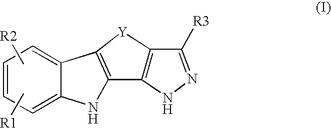

wherein

R1 and R2, being the same or different, are independently hydrogen or halogen atom, nitro, cyano, hydroxy, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl arylaminocarbonyl, heteroarylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl, aryl C₁-C₆ alkyl group, aryl C₁-C₆ alkyloxy group, aryloxy, heteroaryl, heteroaryl C₁-C₆ alkyl group, a straight or branched C₁-C₆ alkylthio, C₁-C₆ alkylsulphinyl, C₁-C₆ alkylsulphonyl, arylthio, arylsulphinyl, arylsulphonyl, C₁-C₆alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆alkylaminosulfonyl and arylaminosulfonyl group;

Y is a —(CH₂)ₙ— group wherein n is 1, 2 or 3, or a carbon-carbon double bond (—CH₂═CH₂—);

R3 is hydrogen atom, cyano, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, an aryl, an aryl C₁-C₆ alkyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl C₁-C₆ alkyloxy group, aryloxy, a straight or branched C₁-C₆ alkylthio, arylC₁-C₆ alkylthio, C₁-C₆ alkylsulphinyl group, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, an ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆ alkylaminosulfonyl, and arylaminosulfonyl group, with the proviso that when R2 and R3 are both hydrogen atoms and Y is a —CH₂—CH₂— group, then R1 is not hydrogen or 7-chloro, 7-bromo atom, 7-cyclohexyl or 7-methyl group, or a pharmaceutically acceptable salt thereof.

In a preferred embodiment of the method described above, the disease caused by and/or associated with an altered protein kinase activity is selected from the group consisting of cancer, cell proliferative disorders, Alzheimer's disease, viral infections, autoimmune diseases and neurodegenerative disorders.

Specific types of cancer that may be treated according to the invention include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderoma pigmentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis. In addition, the method object of the present invention provides tumor angiogenesis and metastasis inhibition.

The tetracyclic pyrazole derivatives of formula (I), object of the invention, are obtainable through a synthetic process comprising well known reactions carried out according to conventional techniques, as well as through a new and extremely versatile solid-phase combinatorial process, being both comprised within the scope of the invention.

The present invention also provides a pharmaceutical composition comprising the tetracyclic pyrazole derivatives of formula (I) with the above proviso and at least one pharmaceutically acceptable excipient, carrier or diluent. A more complete appreciation of the invention and many of the attendant advantages thereof will be readily obtained, as the same becomes better understood by reference to the following detailed description.

### DETAILED DESCRIPTION OF THE INVENTION

The compounds of formula (I), object of the present invention, may have asymmetric carbon atoms and may therefore exist either as racemic admixtures or as individual optical isomers. Accordingly, all the possible isomers and their admixtures and of both the metabolites and the pharmaceutically acceptable bio-precursors (otherwise referred to as pro-drugs) of the compounds of formula (I), as well as any therapeutic method of treatment comprising them, are also within the scope of the present invention.

In addition to the above, as will be readily appreciated, the unsubstituted ring nitrogen pyrazoles in the compounds of the invention are known to rapidly equilibrate, in solution, as admixtures of both tautomers, A and B:

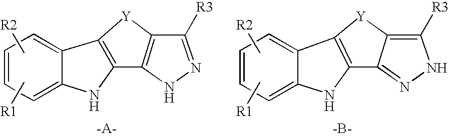

wherein R1, R2, R3 and Y are as defined above.

Accordingly, in the present invention and unless otherwise indicated, where only one tautomer A is indicated for the compounds of formula (I), the other, B, is also within the scope of the present invention.

As used herein, unless otherwise specified, with the term straight or branched C₁-C₈ alkyl, hence also comprising C₁-C₆ alkyl, we intend a group such as, for instance, methyl, ethyl, n-propyl, isopropyl, n-butyl, isobutyl, sec-butyl, tert-butyl, n-pentyl, neopentyl, n-hexyl, isohexyl, n-heptyl, 2-methyl-hexyl-2-yl, n-octyl, and the like.

With the term straight or branched C₂-C₈ alkenyl we intend a group such as, for instance, vinyl, 1- or -2-propenyl, isopropenyl, 1-, 2- or 3-butenyl, pentenyl, hexenyl, heptenyl, octenyl and the like.

With the term saturated or unsaturated C₃-C₇ cycloalkyl or cycloalkyloxy group we intend, for instance, cyclopropyl, cyclobutyl, cyclopentyl, cyclohexyl, cyclopentenyl; cyclohexenyl, cyclopentyloxy, cyclohexyloxy and the like.

With the term aryl we intend an aromatic carbocycle such as, for instance, phenyl, biphenyl, 1-naphthyl, 2-naphthyl, and the like.

With the term heteroaryl we intend an optionally condensed 5 or 6 membered heterocycle with 1 to 4 heteroatoms selected among nitrogen, oxygen or sulphur.

With the term 5 or 6 membered heterocycle with 1 to 4 heteroatoms selected among nitrogen, oxygen or sulphur, we intend a saturated, partly unsaturated or fully unsaturated either aromatic or non aromatic heterocycle such as, for instance, thiophene, furan, pyrrole, imidazole, pyrazole, thiazole, isothiazole, oxazole, isoxazole, pyridine, pyrazine, pyrimidine, pyridazine, pyrrolidine, pyrroline, imidazolidine, imidazoline, piperidine, piperazine, morpholine, tetrahydrofuran, tetrahydropyran, tetrahydrothiopyran, and the like. With the term optionally condensed heterocycle and unless otherwise indicated we intend any of the above defined heterocycles further condensed, through any one of the available bonds, with other heterocycle(s) as defined above or benzene ring(s) such as, for instance, quinoline, isoquinoline, chroman, chromene, thionaphthene, indoline, and the like.

In this respect, unless otherwise indicated, with the term halogen atom we intend a fluorine, chlorine, bromine or iodine atom.

With the term perfluorinated C₁-C₈ alkyl we intend any alkyl group as above defined being substituted by two or more fluorine atoms such as, for instance, trifluoromethyl, 2,2,2-trifluoroethyl, 1,1-difluoroethyl, and the like.

From all of the above, it is clear to the skilled man that any of the groups or substituents being defined, for instance, as arylalkyl, heteroaryllalkyl, alkylaryl, alkoxy, alkoxyalkyloxy, arylalkyloxy, alkylaminocarbonyl, heteroarylcarbonyl, alkylamino, arylamino, alkylthio, arylthio, alkylsulphonyl, arylsulphonyl and the like, have to be construed from the names of the groups from which they originate. As an example, unless specifically noted otherwise, any arylalkyloxycarbonylamino group has to be intended as a carbonylamino group being substituted by alkyloxy wherein the alkyl moiety is further substituted by aryl, both aryl and alkyl being as above defined.

The term “optionally substituted” means that the group may be substituted or unsubstituted; the substituents which may be present in the groups in any of the above definitions of R1-R3 include the following:

halo (i.e., fluoro, bromo, chloro or iodo);

hydroxy;

nitro;

azido;

mercapto (i.e., —SH), and acetyl or phenylacetyl esters thereof (i.e., —SCOCH₃ and —SCOCH₂C₆H₅);

amino (i.e., —NH₂ or —NHR^{I} or —NR^{I}R^{II}, wherein R^{I} and R^{II}, which are the same or different, are straight or branched C₁-C₆ alkyl, phenyl, biphenyl (i.e., —C₆H₄—C₆H₅), or benzyl groups, optionally substituted by hydroxy, methoxy, methyl, amino, methylamino, dimethylamino, chloro or fluoro; or R^{I} and R^{II} taken together with the nitrogen atom to which they are attached form a heterocyclic ring such as morpholino, pyrrolidino, piperidino, pyperazino or N-methylpyperazino;

guanidino, i.e., —NHC(═NH)NH₂;

formyl (i.e. —CHO);

cyano;

carboxy (i.e. —COOH), or esters thereof (i.e., —COOR^{I}), or amides thereof (i.e., —CONH₂, —CONHR^{I} or —CONHR^{I}R^{II}), wherein R^{I} and R^{II} are as defined above, and including morpholino-amides, pyrrolidino-amides, and carboxymethylamides —CONHCH₂COOH;

sulfo (i.e., —SO₃H);

acyl, i.e., —C(O)R^{I}, wherein R^{I} is as defined above, including monofluoroacetyl, difluoroacetyl, trifluoroacetyl;

carbamoyloxy (i.e., —OCONH₂) and N-methylcarbamoyloxy;

acyloxy, i.e., —OC(O)R^{I} wherein R^{I} is as defined above, or formyloxy;

acylamino, i.e., —NHC(O)R^{I}, or —NHC(O)OR^{I}, wherein R^{I} is as defined above or is a group —(CH₂)ₜCOOH where t is 1, 2 or 3;

ureido, i.e., —NH(CO)NH₂, —NH(CO)NHR^{I}, —NH(CO)NR^{I}R^{II}, wherein R^{I} and R^{II} are as defined above, including —NH(CO)-(4-morpholino), —NH(CO)-(1-pyrrolidino), —NH(CO)-(1-piperazino), —NH(CO)-(4-methyl-1-piperazino);

sulfonamido, i.e., —NHSO₂R^{I} wherein R^{I} is as defined above;

a group —(CH₂)ₜCOOH, and esters and amides thereof, i.e.,

(CH₂)ₜCOOR^{I} and —(CH₂)ₜCONH₂, —(CH₂)ₜCONHR^{I}, —(CH₂)ₜCONR^{I}R^{II}, wherein t, R^{I} and R^{II} are as defined above;

a group —NH(SO₂)NH₂, —NH(SO₂)NHR^{II}, —NH(SO₂)NR^{I}R^{II}, wherein R^{I} and R^{II} are as defined above, including —NH(SO₂)-(4-morpholino), —NH(SO₂)-(1-pyrrolidino), —NH(SO₂)-(1-piperazino), —NH(SO₂)-(4-methyl-1-piperazino);

a group —OC(O)OR^{I}, wherein R^{I} is as defined above;

a group —OR^{I}, wherein R^{I} is as defined above, including —OCH₂COOH;

a group —O—CH₂—O—, methylendioxy or —O—CH₂—CH₂—O—, ethylendioxy;

a group —SR^{I}, wherein R^{I} is as defined above, including —SCH₂COOH;

a group —S(O)R^{I}, wherein R^{I} is as defined above;

a group —S(O₂)R^{I}, wherein R^{I} is as defined above;

a group —SO₂NH₂, —SO₂NHR^{I}, or —SO₂NR^{I}R^{II}, wherein R^{I} and R^{II} are as defined above;

C₁-C₆ alkyl or C₂-C₆ alkenyl;

C₃-C₇ cycloalkyl;

substituted methyl selected from chloromethyl, fluoromethyl, difluoromethyl, trifluoromethyl, aminomethyl, N,N-dimethylaminomethyl, azidomethyl, cyanomethyl, carboxymethyl, sulfomethyl, carbamoylmethyl, carbamoyloxymethyl, hydroxymethyl, methoxycarbonylmethyl, ethoxycarbonylmethyl, tert-butoxycarbonylmethyl and guanidinomethyl.

Most preferred substituents are methoxy, trifluoromethyl, methylendioxy, dimethylamino, and ethoxycarbonyl groups.

When present, carboxy, hydroxy, mercapto and amino groups may be either free or in a protected form. Protected forms of said groups are any of those generally known in the art. Preferably, carboxy groups are protected as esters thereof, in particular methyl, ethyl, tert-butyl, benzyl, and 4-nitrobenzyl esters. Preferably, hydroxy groups are protected as silyl-ethers, ethers or esters thereof, in particular trimethyl silyl, tert-butyldiphenyl silyl, triethyl silyl, triisopropyl silyl or tert-butyldimethylsilyl ethers, methoxymethyl ethers, tetrahydropyranyl ethers, benzyl ethers, acetates or benzoates. Preferably, mercapto groups are protected as thioethers or thioesters, in particular tert-butyl thioethers, thioacetates or thiobenzoates. Preferably, amino groups are protected as carbamates, e.g. tert-butoxycarbonyl derivatives, or as amides, e.g. acetamides and benzamides.

Furthermore, hydrates, solvates of compounds of formula (I) are included within the scope of the present invention. With the term oxo we intend a carbonyl (>C═O) group. Pharmaceutically acceptable salts of the compounds of formula (I) are the acid addition salts with inorganic or organic, e.g. nitric, hydrochloric, hydrobromic, sulphuric, perchloric, phosphoric, acetic, trifluoroacetic, propionic, glycolic, lactic, oxalic, malonic, malic, maleic, tartaric, citric, benzoic, cinnamic, mandelic, methanesulphonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

Preferred compounds of formula (I) are the compounds wherein R1 is hydrogen or halogen atom, cyano or hydroxy group, or an optionally substituted group selected from a straight or branched optionally substituted C₁-C₈ alkyl group, a perfluorinated C₁-C₈ alkyl and a saturated or unsaturated C₃-C₇ cycloalkyl group; R2 is hydrogen or halogen atom, cyano, hydroxy, carboxy, or an optionally substituted group selected from aminocarbonyl, amino, hydroxyaminocarbonyl, sulfonamido, ureido, thioureido group, a straight or branched C₁-C₈ alkyl group, a perfluorinated C₁-C₈ alkyl, a saturated or unsaturated C₃-C₇ cycloalkyl, C₁-C₆ alkyloxycarbonyl, aryloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkoxyaminocarbonyl, aryloxyaminocarbonyl, C₁-C₆ alkylcarbonyloxy arylcarbonyloxy, C₁-C₆ alkylamino, arylamino, aryl C₁-C₆ alkylamino, heteroarylamino, heteroarylC₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, C₁-C₆ alkylaminocarbonylamino, aryl C₁-C₆ alkylaminocarbonylamino, C₁-C₆ dialkylaminocarbonylamino, arylaminocarbonylamino, C₁-C₆ alkylsulfonylamino, arylsulfonylamino, C₁-C₆ alkylaminosulfonyl, straight or branched C₁-C₆ alkylthio and arylaminosulfonyl group;

Y is a —(CH₂)ₙ— group, wherein n is 1, 2 or 3, or a carbon-carbon double bond —CH═CH—;

R3 is hydrogen atom, carboxy or an optionally substituted group selected from C₁-C₆ straight or branched alkyl, perfluorinated C₁-C₆ alkyl, aryl C₁-C₆ alkyl group, C₁-C₆ alkyloxycarbonyl, aryl C₁-C₆ alkyloxycarbonyl, straight or branched C₁-C₆ alkylthio, C₁-C₆ alkylaminocarbonyl, C₁-C₆ dialkylaminocarbonyl, arylaminocarbonyl and aryl C₁-C₆ alkylaminocarbonyl, with the proviso that when R2 and R3 are both hydrogen atoms and Y is a —CH₂—CH₂— group, then R1 is not hydrogen, 7-chloro or 7-bromo atom or 7-cyclohexyl or 7-methyl group, or a pharmaceutically acceptable salt thereof.

Still more preferred, within this class, are the 1,4,5,10-tetrahydropyrazolo[3,4-a]carbazole derivatives of formula (IA)

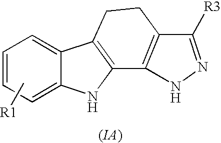

wherein R1 is halogen atom, cyano, nitro, hydroxy, carboxy, aminocarbonyl, hydroxyaminocarbonyl, amino or sulfonamido group, or an optionally substituted group selected from a straight or branched C₁-C₈ alkyl group, a perfluorinated C₁-C₈ alkyl, a saturated or unsaturated C₃-C₇ cycloalkyl, a straight or branched C₁-C₈ alkoxy group, alkyloxycarbonyl, aryloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkoxyaminocarbonyl, aryloxyaminocarbonyl, C₁-C₆ alkylcarbonyloxy, arylcarbonyloxy, an C₁-C₆ alkylamino, arylamino, aryl C₁-C₆ alkylamino, C₁-C₆ alkylcarbonylamino, arylcarbonylamino, aryloxycarbonylamino, C₁-C₆ alkylaminocarbonylamino, C₁-C₆ dialkylaminocarbonylamino, aryl C₁-C₆ alkylaminocarbonylamino, arylaminocarbonylamino, C₁-C₆ alkylsulfonylamino, arylsulfonylamino, C₁-C₆ alkylaminosulfonyl and arylaminosulfonyl;

R3 is hydrogen atom, a carboxy group or an optionally substituted group selected from C₁-C₆ straight or branched alkyl, C₁-C₆ alkyloxycarbonyl, aryl C₁-C₆ alkyloxycarbonyl, C₁-C₆ alkylaminocarbonyl, C₁-C₆ dialkylaminocarbonyl, arylaminocarbonyl and aryl C₁-C₆ alkylaminocarbonyl, with the proviso that when R3 is hydrogen atom, then R1 is not hydrogen or 7-chloro, 7-bromo atom, 7-cyclohexyl or 7-methyl group, or a pharmaceutically acceptable salt thereof.

For clarity, we point out that the framework of the preferred compounds of formula (IA) of the present invention is numbered as follows:

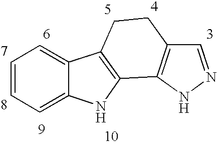

Specific, not limiting, preferred compounds of formula (IA) of the invention, whenever appropriate in the form of pharmaceutically acceptable salts, are the following:

1 6-fluoro-2,4,5,10-tetrahydropyrazolo[3,4-a]carbazole;

2 7-fluoro-2,4,5,10-tetrahydropyrazolo[3,4-a]carbazole;

3 8-fluoro-2,4,5,10-tetrahydropyrazolo[3,4-a]carbazole;

4 6-chloro-2,4,5,10-tetrahydropyrazolo[3,4-a]carbazole;

5 8-chloro-2,4,5,10-tetrahydropyrazolo[3,4-a]carbazole;

6 6-bromo-2,4,5,10-tetrahydropyrazolo[3,4-a]carbazole;

7 8-bromo-2,4,5,10-tetrahydropyrazolo[3,4-a]carbazole;

8 6-cyano-2,4,5,10-tetrahydropyrazolo[3,4-a]carbazole;

9 7-cyano-2,4,5,10-tetrahydropyrazolo[3,4-a]carbazole;

10 8-cyano-2,4,5,10-tetrahydropyrazolo[3,4-a]carbazole;

11 6-nitro-2,4,5,10-tetrahydropyrazolo[3,4-a]carbazole;

12 7-nitro-2,4,5,10-tetrahydropyrazolo[3,4-a]carbazole;

13 8-nitro-2,4,5,10-tetrahydropyrazolo[3,4-a]carbazole;

14 6-methyl-2,4,5,10-tetrahydropyrazolo[3,4-a]carbazole;

15 8-methyl-2,4,5,10-tetrahydropyrazolo[3,4-a]carbazole;

16 6-trifluoromethyl-2,4,5,10-tetrahydropyrazolo[3,4-a]carbazole;

17 7-trifluoromethyl-2,4,5,10-tetrahydropyrazolo[3,4-a]carbazole;

18 8-trifluoromethyl-2,4,5,10-tetrahydropyrazolo[3,4-a]carbazole;

19 6-methoxy-2,4,5,10-tetrahydropyrazolo[3,4-a]carbazole;

20 7-methoxy-2,4,5,10-tetrahydropyrazolo[3,4-a]carbazole;

21 8-methoxy-2,4,5,10-tetrahydropyrazolo[3,4-a]carbazole;

22 6-hydroxy-2,4,5,10-tetrahydropyrazolo[3,4-a]carbazole;

23 7-hydroxy-2,4,5,10-tetrahydropyrazolo[3,4-a]carbazole;

24 8-hydroxy-2,4,5,10-tetrahydropyrazolo[3,4-a]carbazole;

25 2,4,5,10-tetrahydropyrazolo[3,4-a]carbazole-6-carboxylic acid;

26 2,4,5,10-tetrahydropyrazolo[3,4-a]carbazole-7-carboxylic acid;

27 2,4,5,10-tetrahydropyrazolo[3,4-a]carbazole-8-carboxylic acid;

28 methyl 2,4,5,10-tetrahydropyrazolo[3,4-a]carbazole-6-carboxylate;

29 methyl 2,4,5,10-tetrahydropyrazolo[3,4-a]carbazole-7-carboxylate;

30 methyl 2,4,5,10-tetrahydropyrazolo[3,4-a]carbazole-8-carboxylate;

31 ethyl 2,4,5,10-tetrahydropyrazolo[3,4-a]carbazole-6-carboxylate;

32 ethyl 2,4,5,10-tetrahydropyrazolo[3,4-a]carbazole-7-carboxylate;

33 ethyl 2,4,5,10-tetrahydropyrazolo[3,4-a]carbazole-8-carboxylate;

34 i-butyl 2,4,5,10-tetrahydropyrazolo[3,4-a]carbazole-6-carboxylate;

35 i-butyl 2,4,5,10-tetrahydropyrazolo[3,4-a]carbazole-7-carboxylate;

36 i-butyl 2,4,5,10-tetrahydropyrazolo[3,4-a]carbazole-8-carboxylate;

37 1,4,5,10-tetrahydropyrazolo[3,4-a]carbazole-3-carboxylic acid

38 methyl 2,4,5,10-tetrahydropyrazolo[3,4-a]carbazole-3-carboxylate;

39 ethyl 2,4,5,10-tetrahydropyrazolo[3,4-a]carbazole-3-carboxylate;

40 propyl 2,4,5,10-tetrahydropyrazolo[3,4-a]carbazole-3-carboxylate;

41 i-propyl 2,4,5,10-tetrahydropyrazolo[3,4-a]carbazole-3-carboxylate;

42 butyl 2,4,5,10-tetrahydropyrazolo[3,4-a]carbazole-3-carboxylate;

43 i-butyl 2,4,5,10-tetrahydropyrazolo[3,4-a]carbazole-3-carboxylate;

44 1,4,5,10-tetrahydropyrazolo[3,4-a]carbazole-6-carboxamide;

45 1,4,5,10-tetrahydropyrazolo[3,4-a]carbazole-7-carboxamide;

46 1,4,5,10-tetrahydropyrazolo[3,4-a]carbazole-8-carboxamide;

47 N-methyl-1,4,5,10-tetrahydropyrazolo[3,4-a]carbazole-6-carboxamide;

48 N-methyl-1,4,5,10-tetrahydropyrazolo[3,4-a]carbazole-7-carboxamide;

49 N-methyl-1,4,5,10-tetrahydropyrazolo[3,4-a]carbazole-8-carboxamide;

50 N-ethyl-1,4,5,10-tetrahydropyrazolo[3,4-a]carbazole-6-carboxamide;

51 N-ethyl-1,4,5,10-tetrahydropyrazolo[3,4-a]carbazole-7-carboxamide;

52 N-ethyl-1,4,5,10-tetrahydropyrazolo[3,4-a]carbazole-8-carboxamide;

53 N-propyl-1,4,5,10-tetrahydropyrazolo[3,4-a]carbazole-6-carboxamide;

54 N-propyl-1,4,5,10-tetrahydropyrazolo[3,4-a]carbazole-7-carboxamide;

55 N-propyl-1,4,5,10-tetrahydropyrazolo[3,4-a]carbazole-8-carboxamide;

56 N-isopropyl-1,4,5,10-tetrahydropyrazolo[3,4-a]carbazole-6-carboxamide;

57 N-isopropyl-1,4,5,10-tetrahydropyrazolo[3,4-a]carbazole-7-carboxamide;

58 N-isopropyl-1,4,5,10-tetrahydropyrazolo[3,4-a]carbazole-8-carboxamide;

59 N-butyl-1,4,5,10-tetrahydropyrazolo[3,4-a]carbazole-6-carboxamide;

60 N-butyl-1,4,5,10-tetrahydropyrazolo[3,4-a]carbazole-7-carboxamide;

61 N-butyl-1,4,5,10-tetrahydropyrazolo[3,4-a]carbazole-8-carboxamide;

62 N-isobutyl-1,4,5,10-tetrahydropyrazolo[3,4-a]carbazole-6-carboxamide;

63 N-isobutyl-1,4,5,10-tetrahydropyrazolo[3,4-a]carbazole-7-carboxamide;

64 N-isobutyl-1,4,5,10-tetrahydropyrazolo[3,4-a]carbazole-8-carboxamide;

65 N-terbutyl-1,4,5,10-tetrahydropyrazolo[3,4-a]carbazole-6-carboxamide;

66 N-terbutyl-1,4,5,10-tetrahydropyrazolo[3,4-a]carbazole-7-carboxamide;

67 N-terbutyl-1,4,5,10-tetrahydropyrazolo[3,4-a]carbazole-8-carboxamide;

68 N-phenyl-1,4,5,10-tetrahydropyrazolo[3,4-a]carbazole-6-carboxamide;

69 N-phenyl-1,4,5,10-tetrahydropyrazolo[3,4-a]carbazole-7-carboxamide;

70 N-phenyl-1,4,5,10-tetrahydropyrazolo[3,4-a]carbazole-8-carboxamide;

71 N-benzyl-1,4,5,10-tetrahydropyrazolo[3,4-a]carbazole-6-carboxamide;

72 N-benzyl-1,4,5,10-tetrahydropyrazolo[3,4-a]carbazole-7-carboxamide;

73 N-benzyl-1,4,5,10-tetrahydropyrazolo[3,4-a]carbazole-8-carboxamide;

74 N-(3-dimethylamino)propyl-1,4,5,10-tetrahydropyrazolo[3,4-a]carbazole-6-carboxamide;

75 N-(3-dimethylamino)propyl-1,4,5,10-tetrahydropyrazolo[3,4-a]carbazole-7-carboxamide;

76 N-(3-dimethylamino)propyl-1,4,5,10-tetrahydropyrazolo[3,4-a]carbazole-8-carboxamide;

77 N-(3-dimethylamino)propyl-1,10-dihydropyrazolo[3,4-a]carbazole-6-carboxamide;

78 N-(3-dimethylamino)propyl-1,10-dihydropyrazolo[3,4-a]carbazole-7-carboxamide;

79 N-(3-dimethylamino)propyl-1,10-dihydropyrazolo[3,4-a]carbazole-8-carboxamide;

80 N-(5-hydroxy-1H-pyrazol-3-yl)-1,4,5,10-tetrahydropyrazolo[3,4-a]carbazole-6-carboxamide;

81 N-(5-hydroxy-1H-pyrazol-3-yl)-1,4,5,10-tetrahydropyrazolo[3,4-a]carbazole-7-carboxamide;

82 N-(5-hydroxy-1H-pyrazol-3-yl)-1,4,5,10-tetrahydropyrazolo[3,4-a]carbazole-8-carboxamide;

83 N-(5-hydroxy-1H-pyrazol-3-yl)-1,10-dihydropyrazolo[3,4-a]carbazole-6-carboxamide;

84 N-(5-hydroxy-1H-pyrazol-3-yl)-1,10-dihydropyrazolo[3,4-a]carbazole-7-carboxamide;

85 N-(5-hydroxy-1H-pyrazol-3-yl)-1,10-dihydropyrazolo[3,4-a]carbazole-8-carboxamide;

86 N-(3-morpholin-4-yl-propyl)-1,4,5,10-tetrahydropyrazolo[3,4-a]carbazole-6-carboxamide;

87 N-(3-morpholin-4-yl-propyl)-1,4,5,10-tetrahydropyrazolo[3,4-a]carbazole-7-carboxamide;

88 N-(3-morpholin-4-yl-propyl)-1,4,5,10-tetrahydropyrazolo[3,4-a]carbazole-8-carboxamide;

89 N-(3-morpholin-4-yl-propyl)-1,10-dihydropyrazolo[3,4-a]carbazole-6-carboxamide;

90 N-(3-morpholin-4-yl-propyl)-1,10-dihydropyrazolo[3,4-a]carbazole-7-carboxamide;

91 N-(3-morpholin-4-yl-propyl)-1,10-dihydropyrazolo[3,4-a]carbazole-8-carboxamide;

92 N-(2-phenylamino-ethyl)-1,4,5,10-tetrahydropyrazolo[3,4-a]carbazole-6-carboxamide;

93 N-(2-phenylamino-ethyl)-1,4,5,10-tetrahydropyrazolo[3,4-a]carbazole-7-carboxamide;

94 N-(2-phenylamino-ethyl)-1,4,5,10-tetrahydropyrazolo[3,4-a]carbazole-8-carboxamide;

95 N-(2-phenylamino-ethyl)-1,10-dihydropyrazolo[3,4-a]carbazole-6-carboxamide;

96 N-(2-phenylamino-ethyl)-1,10-dihydropyrazolo[3,4-a]carbazole-7-carboxamide;

97 N-(2-phenylamino-ethyl)-1,10-dihydropyrazolo[3,4-a]carbazole-8-carboxamide;

98 N-[2-(1H-imidazol-4-yl)-ethyl]-1,4,5,10-tetrahydropyrazolo[3,4-a]carbazole-6-carboxamide;

99 N-[2-(1H-imidazol-4-yl)-ethyl]-1,4,5,10-tetrahydropyrazolo[3,4-a]carbazole-7-carboxamide;

100 N-[2-(1H-imidazol-4-yl)-ethyl]-1,4,5,10-tetrahydropyrazolo[3,4-a]carbazole-8-carboxamide;

101 N-[2-(1H-imidazol-4-yl)-ethyl]-1,10-dihydropyrazolo[3,4-a]carbazole-6-carboxamide;

102 N-[2-(1H-imidazol-4-yl)-ethyl]-1,10-dihydropyrazolo[3,4-a]carbazole-7-carboxamide;

103 N-[2-(1H-imidazol-4-yl)-ethyl]-1,10-dihydropyrazolo[3,4-a]carbazole-8-carboxamide;

104 N-(4-hydroxy-butyl)-1,4,5,10-tetrahydropyrazolo[3,4-a]carbazole-6-carboxamide;

105 N-(4-hydroxy-butyl)-1,4,5,10-tetrahydropyrazolo[3,4-a]carbazole-7-carboxamide;

106 N-(4-hydroxy-butyl)-1,4,5,10-tetrahydropyrazolo[3,4-a]carbazole-8-carboxamide;

107 N-(4-hydroxy-butyl)-1,10-dihydropyrazolo[3,4-a]carbazole-6-carboxamide;

108 N-(4-hydroxy-butyl)-1,10-dihydropyrazolo[3,4-a]carbazole-7-carboxamide;

109 N-(4-hydroxy-butyl)-1,10-dihydropyrazolo[3,4-a]carbazole-8-carboxamide;

110 N-(2-hydroxymethyl-phenyl)-1,4,5,10-tetrahydropyrazolo[3,4-a]carbazole-6-carboxamide;

111 N-(2-hydroxymethyl-phenyl)-1,4,5,10-tetrahydropyrazolo[3,4-a]carbazole-7-carboxamide;

112 N-(2-hydroxymethyl-phenyl)-1,4,5,10-tetrahydropyrazolo[3,4-a]carbazole-8-carboxamide;

113 N-(2-hydroxymethyl-phenyl)-1,10-dihydropyrazolo[3,4-a]carbazole-6-carboxamide;

114 N-(2-hydroxymethyl-phenyl)-1,10-dihydropyrazolo[3,4-a]carbazole-7-carboxamide;

115 N-(2-hydroxymethyl-phenyl)-1,10-dihydropyrazolo[3,4-a]carbazole-8-carboxamide;

116 N-(furan-2-ylmethyl)-1,4,5,10-tetrahydropyrazolo[3,4-a]carbazole-6-carboxamide;

117 N-(furan-2-ylmethyl)-1,4,5,10-tetrahydropyrazolo[3,4-a]carbazole-7-carboxamide;

118 N-(furan-2-ylmethyl)-1,4,5,10-tetrahydropyrazolo[3,4-a]carbazole-8-carboxamide;

119 N-(furan-2-ylmethyl)-1,10-dihydropyrazolo[3,4-a]carbazole-6-carboxamide;

120 N-(furan-2-ylmethyl)-1,10-dihydropyrazolo[3,4-a]carbazole-7-carboxamide;

121 N-(furan-2-ylmethyl)-1,10-dihydropyrazolo[3,4-a]carbazole-8-carboxamide;

122 N-(pyridin-4-ylmethyl)-1,4,5,10-tetrahydropyrazolo[3,4-a]carbazole-6-carboxamide;

123 N-(pyridin-4-ylmethyl)-1,4,5,10-tetrahydropyrazolo[3,4-a]carbazole-7-carboxamide;

124 N-(pyridin-4-ylmethyl)-1,4,5,10-tetrahydropyrazolo[3,4-a]carbazole-8-carboxamide;

125 N-(pyridin-4-ylmethyl)-1,10-dihydropyrazolo[3,4-a]carbazole-6-carboxamide;

126 N-(pyridin-4-ylmethyl)-1,10-dihydropyrazolo[3,4-a]carbazole-7-carboxamide;

127 N-(pyridin-4-ylmethyl)-1,10-dihydropyrazolo[3,4-a]carbazole-8-carboxamide;

128 N-[(methoxycarbonyl)methyl]-1,4,5,10-tetrahydropyrazolo[3,4-a]carbazole-6-carboxamide;

129 N-[(methoxycarbonyl)methyl]-1,4,5,10-tetrahydropyrazolo[3,4-a]carbazole-7-carboxamide;

130 N-[(methoxycarbonyl)methyl]-1,4,5,10-tetrahydropyrazolo[3,4-a]carbazole-8-carboxamide;

131 N-[(methoxycarbonyl)methyl]-1,10-dihydropyrazolo[3,4-a]carbazole-6-carboxamide;

132 N-[(methoxycarbonyl)methyl]-1,10-dihydropyrazolo[3,4-a]carbazole-7-carboxamide;

133 N-[(methoxycarbonyl)methyl]-1,10-dihydropyrazolo[3,4-a]carbazole-8-carboxamide;

134 N-(ethane-2-sulfonic acid)-1,4,5,10-tetrahydropyrazolo[3,4-a]carbazole-6-carboxamide;

135 N-(ethane-2-sulfonic acid)-1,4,5,10-tetrahydropyrazolo[3,4-a]carbazole-7-carboxamide;

136 N-(ethane-2-sulfonic acid)-1,4,5,10-tetrahydropyrazolo[3,4-a]carbazole-8-carboxamide;

137 7-[(4-methylpiperazin-1-yl)carbonyl]-1,4,5,10-tetrahydropyrazolo[3,4-a]carbazole;

138 1,4,5,10-tetrahydropyrazolo[3,4-a]carbazol-6-amine;

139 1,4,5,10-tetrahydropyrazolo[3,4-a]carbazol-7-amine;

140 1,4,5,10-tetrahydropyrazolo[3,4-a]carbazol-8-amine;

141 N-(1,4,5,10-tetrahydropyrazolo[3,4-a]carbazol-6-yl)acetamide;

142 N-(1,4,5,10-tetrahydropyrazolo[3,4-a]carbazol-7-yl)acetamide;

143 N-(1,4,5,10-tetrahydropyrazolo[3,4-a]carbazol-8-yl)acetamide;

144 N-(1,4,5,10-tetrahydropyrazolo[3,4-a]carbazol-6-yl)propanamide;

145 N-(1,4,5,10-tetrahydropyrazolo[3,4-a]carbazol-7-yl)propanamide;

146 N-(1,4,5,10-tetrahydropyrazolo[3,4-a]carbazol-8-yl)propanamide;

147 2-methyl-N-(1,4,5,10-tetrahydropyrazolo[3,4-a]carbazol-6-yl)propanamide;

148 2-methyl-N-(1,4,5,10-tetrahydropyrazolo[3,4-a]carbazol-7-yl)propanamide;

149 2-methyl-N-(1,4,5,10-tetrahydropyrazolo[3,4-a]carbazol-8-yl)propanamide;

150 N-(1,4,5,10-tetrahydropyrazolo[3,4-a]carbazol-6-yl)butanamide;

151 N-(1,4,5,10-tetrahydropyrazolo[3,4-a]carbazol-7-yl)butanamide;

152 N-(1,4,5,10-tetrahydropyrazolo[3,4-a]carbazol-8-yl)butanamide;

153 N-(1,4,5,10-tetrahydropyrazolo[3,4-a]carbazol-6-yl)benzamide;

154 N-(1,4,5,10-tetrahydropyrazolo[3,4-a]carbazol-7-yl)benzamide;

155 N-(1,4,5,10-tetrahydropyrazolo[3,4-a]carbazol-8-yl)benzamide;

156 N-(1,4,5,10-tetrahydropyrazolo[3,4-a]carbazol-6-yl)phenylacetamide;

157 N-(1,4,5,10-tetrahydropyrazolo[3,4-a]carbazol-7-yl)phenylacetamide;

158 N-(1,4,5,10-tetrahydropyrazolo[3,4-a]carbazol-8-yl)phenylacetamide;

159 3-methyl-N-(1,4,5,10-tetrahydropyrazolo[3,4-a]carbazol-6-yl)butanamide;

160 3-methyl-N-(1,4,5,10-tetrahydropyrazolo[3,4-a]carbazol-7-yl)butanamide;

161 3-methyl-N-(1,4,5,10-tetrahydropyrazolo[3,4-a]carbazol-8-yl)butanamide;

162 N-(2,4,5,10-tetrahydropyrazolo[3,4-a]carbazol-7-yl)thiophene-2-carboxamide;

163 N-methyl-N′-(1,4,5,10-tetrahydropyrazolo[3,4-a]carbazol-7-yl)urea;

164 N-propyl-N′-(1,4,5,10-tetrahydropyrazolo[3,4-a]carbazol-7-yl)urea;

165 N-benzyl-N′-(1,4,5,10-tetrahydropyrazolo[3,4-a]carbazol-7-yl)urea;

166 N-phenyl-N′-(1,4,5,10-tetrahydropyrazolo[3,4-a]carbazol-7-yl)urea;

167 4,9-dihydro-1H-pyrazolo[4′,3′:4,5]cyclopenta[1,2-b]indole-6-carboxamide;

168 N-(4,9-dihydro-1H-pyrazolo[4′,3′:4,5]cyclopenta[1,2-b]indol-6-yl)acetamide;

169 N-(4,9-dihydro-1H-pyrazolo[4′,3′:4,5]cyclopenta[1,2-b]indol-6-yl)-3-methylbutanamide;

170 N-(4,9-dihydro-1H-pyrazolo[4′,3′:4,5]cyclopenta[1,2-b]indol-6-yl)-2-phenylacetamide;

171 6-chloro-4,9-dihydro-1H-pyrazolo[4′,3′:4,5]cyclopenta[1,2-b]indole;

172 N-isobutyl-4,9-dihydro-1H-pyrazolo[4′,3′:4,5]cyclopenta[1,2-b]indole-6-carboxamide;

173 N-benzyl-4,9-dihydro-1H-pyrazolo[4′,3′:4,5]cyclopenta[1,2-b]indole-6-carboxamide;

174 ethyl 4,9-dihydro-1H-pyrazolo[4′,3′:4,5]cyclopenta [1,2-b]indole-3-carboxylate;

175 4,5,6,11-tetrahydro-1H-pyrazolo[4′,3′:6,7]cyclohepta [1,2-b]indole-8-carboxamide;

176 3-methyl-N-(4,5,6,11-tetrahydro-1H-pyrazolo[4′,3′:6,7]cyclohepta[1,2-b]indol-8-yl)butanamide;

177 8-chloro-4,5,6,11-tetrahydro-1H-pyrazolo[4′,3′:6,7]cyclohepta[1,2-b]indole;

178 N-benzyl-4,5,6,11-tetrahydro-1H-pyrazolo[4′,3′:6,7]cyclohepta[1,2-b]indole-8-carboxamide;

179 N-isobutyl-4,5,6,11-tetrahydro-1H-pyrazolo[4′,3′:6,7]cyclohepta[1,2-b]indole-8-carboxamide;

180 ethyl 4,5,6,11-tetrahydro-1H-pyrazolo[4′,3′:6,7]cyclohepta[1,2-b]indole-3-carboxylate;

181 N-(4,5,6,11-tetrahydro-1H-pyrazolo[4′,3′:6,7]cyclohepta [1,2-b]indol-8-yl)acetamide;

182 2-phenyl-N-(4,5,6,11-tetrahydro-1H-pyrazolo[4′,3′:6,7]cyclohepta[1,2-b]indol-8-yl)acetamide and

183 3-methylsulfanyl-1,4,5,10-tetrahydropyrazolo[3,4-a]carbazole.

As set forth above, the processes for preparing the compounds of formula (I) and the pharmaceutically acceptable salts thereof are further objects of the present invention.

In a further aspect, the present invention also provides a process for preparing a compound of formula (I), which process comprises:

i) treating a compound of formula (VII) 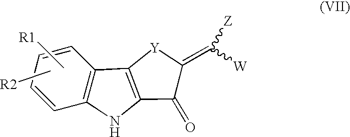
wherein Y is —(CH₂)ₙ—; n, R1 and R2 are as above defined; W and Z have, respectively, one the following couple of meanings:
a) W is a dialkylamino group, and Z is a hydrogen atom;
b) W is a hydroxy group, and Z is a hydrogen atom, a C₁-C₄ alkoxycarbonyl group or a methyl group;
c) Z is a C₁-C₆ alkylthio or arylC₁-C₆ alkylthio group, for instance a methylthio or a benzylthio group and W is:
i) a methylthio group,
ii) a substituted or disubstituted amino group, for instance an alkylamino or arylamino group;
iii) a group of general formula —CH(J)(X) where J and X are, the same or different, electron withdrawing groups, such as, for instance, nitrile, alkoxycarbonyl, aryl including heteroaryl groups;
iv) an alkyl or aryl group;
v) an alkyl- or aryl-carbonyl group;
vi) a cyano group or
d) both Z and W are substituted or disubstituted amino groups;

with hydrazine in a suitable solvent such as methanol, ethanol, N,N-dimethyl formamide, dimethoxyethane, 1,4-dioxane and the like, to give a compound of general formula (I) wherein Y is a —(CH₂)ₙ— group, n, R1 and R2 are as described above, and R3 is C₁-C₆ alkylthio or arylC₁-C₆ alkylthio group, a substituted or disubstituted amino group; a group of the formula —CH(J) (X) wherein X and J are, the same or different, electron withdrawing groups, such as, for instance, C₁-C₆ alkoxycarbonyl, aryl including heteroaryl groups; a C₁-C₆ alkyl or aryl group; a C₁-C₆ alkyl- or aryl-carbonyl group; a cyano group and

ii) optionally converting a compound of general formula (I) into a different compound of formula (I), if necessary separating a mixture of a compound of formula (I) wherein Y is a —CH₂—CH₂— group and a compound of formula (I) wherein Y is a —CH═CH— group and, if desired, converting a compound of formula (I) into a pharmaceutically acceptable salt thereof or converting a salt into the free compound (I).

The reaction i) with hydrazine can be carried for example as described in Pharmaceut. Chem J. 1994, 28, 566; at a temperature ranging from 0° C. to 100° C.

In another aspect, the present invention also provides a process for preparing a compound of the formula (I) wherein Y is a carbon-carbon double bond —C═C—, which process comprises:

i) treating with hydrazine a compound of formula (VIIa)

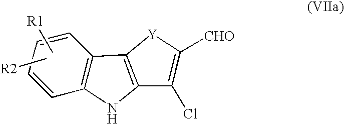

wherein Y is a carbon-carbon double bond —CH═CH—, R1 and R2 are as above defined, to give a compound of general formula (I) wherein Y is a carbon-carbon double bond and R1, R2 are as described above, and R3 is hydrogen atom, and

ii) optionally converting a compound of general formula (I) into a different compound of formula (I) and, if desired, converting a compound of formula (I) into a pharmaceutically acceptable salt thereof or converting a salt into the free compound (I).

The treatment with hydrazine of a compound of formula (VIIa) according to step i) with can be carried out as described for example in Indian J. Chem. 1998, 37B, 314.

It is clear to the person skilled in the art that if a compound of formula (I), prepared according to the above processes, is obtained as an admixture of isomers, their separation into the single isomers of formula (I), carried out according to conventional techniques, is still within the scope of the present invention.

Likewise, the salification of a compound of formula (I) or the conversion of its salt into the free compound (I), carried out according to well-known procedures in the art, are still within the scope of the invention.

The optional conversion of a compound of general formula (I) into a different compound of formula (I) may be carried out in different ways, depending on the desired transformation of the substituents. When in a compound of general formula (I) Y is —CH₂—CH₂— group, the conversion may lead to a mixture of a compound of formula (I) wherein Y is a —CH₂—CH₂— group and a compound of formula (I) wherein Y is a —CH═CH— group, that is, a fully aromatized compound. The two different compounds of formula (I) can be conveniently separated by known chromatographic technique. According to a preferred aspect of the invention, for avoiding the unwanted by-products formation, a compound of formula (I) could be first supported onto a suitable solid support, such as a resin and then, after appropriate reactions for the conversion, cleaved to give a different compound of formula (I). We describe herein below some methods for said conversion, carried out in solution or on solid support.

A) For example, a compound of formula (I) wherein Y is a —(CH₂)ₙ— group, n, R1 and R2 are as described above, R3 is a straight or branched opt. substituted C₁-C₆ alkylthio, aryl C₁-C₆ alkylthio, C₁-C₆ alkylsulphinyl, C₁-C₆ alkylsulphonyl, arylthio, arylsulphinyl or arylsulphonyl group, said compound of formula (I) having optionally protected the pyrazole and cycloalkan [b]indole nitrogen atoms with suitable N-protecting groups, may be reacted with a compound of general formula R-M where R is a suitable aliphatic or aromatic group as defined above for R3, and M represents magnesium halide, zinc halide, boronic acid or alkyl ester, to give a compound of formula (I) wherein Y is a —(CH₂)ₙ— group, n, R1 and R2 are as described above, and R3 represents an aliphatic or aromatic group as defined above.

B) A compound of formula (I) wherein Y is a —(CH₂)ₙ— group, n, R1 and R2 are as described above, R3 is a C₁-C₆ alkylthio or aryl C₁-C₆ alkylthio group, said compound of formula (I) having optionally protected the pyrazole and cycloalkan [b]indole nitrogen atoms with suitable protecting groups, may be oxidised and

B′) the resultant compound of formula (I) is then reacted with an appropriate nucleophile, to give a compound of general formula (I) wherein Y is a —(CH₂)ₙ— group, n, R1 and R2 are as described above and R3 represents:

a) a different alkylthio group;

b) an alkyloxy group;

c) a substituted or disubstituted amino group;

d) a group of general formula —CH(J) (X) where J and X are as above defined;

e) a cyano group.

The conversion under A) above is preferably carried out in the optional presence of a certain amount a transition metal-based salt or complex, such as, for instance, palladium acetate, tetrakis(triphenylphosphine) palladium, palladium chloride, bis(triphenylphosphine) nickel bromide, copper iodide, copper thiophene-2-carboxylate, in the optional presence of an organic base, such as for instance, triethylamine, or an inorganic salt, such as, for instance, caesium fluoride, caesium carbonate, potassium carbonate potassium orthophosphate and the like, in a suitable solvent such as, for instance, tetrahydrofuran, dimethoxyethane or dimethylformamide, using temperature ranging from −20° to 10° C.

The oxidation under B) above can be carried out for instance by means of m-chloroperbenzoic acid, oxone, and the like, in a suitable solvent, for instance dichloromethane, tetrahydrofuran and the like, at a temperature ranging from −20° C. C to the reflux temperature, for a time ranging from 5 minutes to 72 hours. The reaction under B′) above is carried out with an appropriate nucleophile in the proper conditions according to the substituents mentioned above respectively:
a) an alkyl or aryl mercaptane in the presence of a suitable organic or inorganic base, such as, for instance, diisopropyl ethyl amine or potassium carbonate and the like;
b) an alcohol or phenol in the presence of a suitable organic or inorganic base diisopropyl ethyl amine or potassium carbonate and the like;
c) an aliphatic or aromatic primary or secondary amine;
d) a compound of general formula J(CH₂)X where J and X are as above defined, in the presence of a suitable base, for instance sodium hydride in a inert solvent like tetrahydrofuran or dimethylformamide, at temperature ranging from 0° to 100° C. or
e) an inorganic cyanide, such as, for instance, sodium cyanide or copper cyanide.

Other functionality modifications on the final tetracycle for converting a compound of formula (I) into a different compound of formula (I) are reported herein below.

The esterification step of a carboxy pyrazolo[3,4-a]cycloalkan[b]indole derivative of formula (I), the reduction step of a nitro pyrazolo[3,4-a]cycloalkan[b]indole derivative of formula (I) and the hydrolysis of the ester group of alkyl pyrazolo[3,4-a]cycloalkan[b]indole-3-carboxylate of formula (I) are reported in the following scheme, wherein Re is an ester residue and Y, R1, R2 and R3 are as defined above.

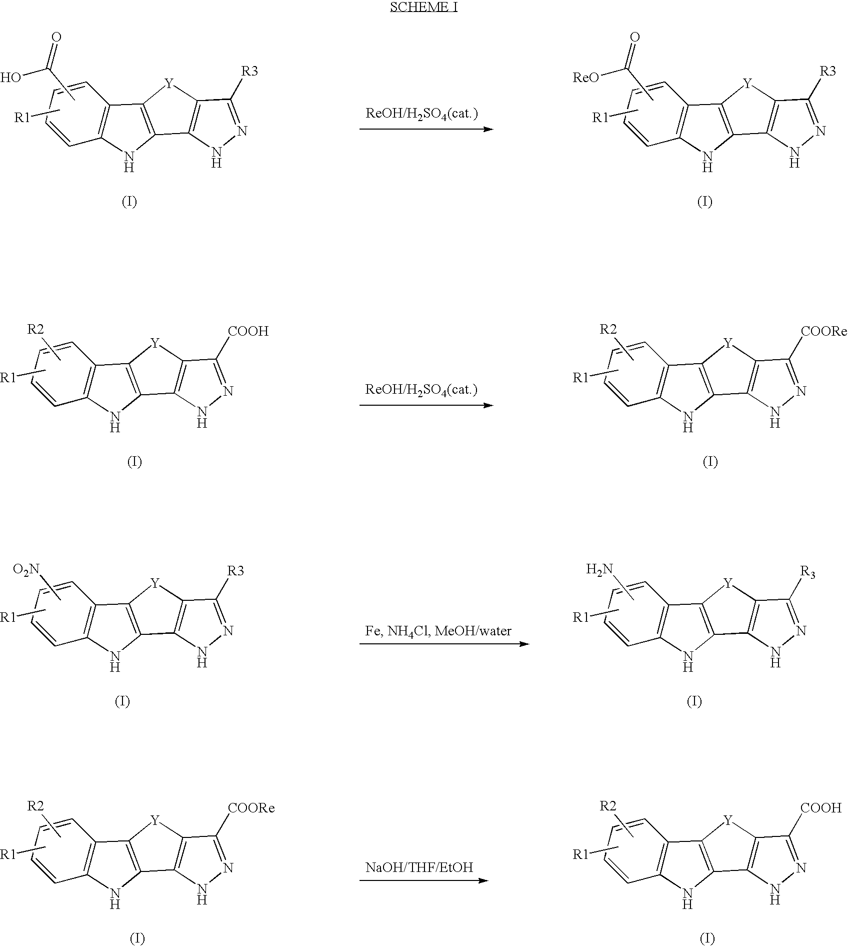

The esterification steps can be performed by standard methods as well as the ester group hydrolysis. The transformation of nitro into amino can be performed by means of well known methods, such as, for instance, chemical reduction with iron or zinc in acids or ammonium chloride or tin (II) chloride. The reaction may occur in a suitable solvent such as, for instance, N,N-dimethylformamide, 1,4-dioxane, ethanol/water, methanol/water, 1-methyl-2-pyrrolidinone or acetonitrile, at a temperature ranging from about −10° C. to reflux and for a suitable time, for instance from about 30 minutes to about 96 hours.

The said reduction may be also performed as a catalytic hydrogenation, according to conventional techniques, in the presence of a suitable catalyst such as, for instance, copper (II) acetate, palladium on charcoal or 4-dimethylaminopyridine.

As said before, the conversion of a compound of formula (I) into a different compound of formula (I) may be preferably carried out on a solid support. That conversion, which is another object of the present invention, may be carried out by reacting a compound of formula (I) as defined above with a suitable activated solid support, then making the desired functionality modifications, and cleaving the resultant compound so as to eliminate the solid support obtaining the desired compound of formula (I).

Some examples of direct functionality modifications on the final tetracycle on solid phase are reported in the following schemes.

For example, in Scheme II the conversion into derivatives of general formula (I) containing acylamines as substituents is shown, wherein Y, R2 and R3 are as described above, L is CO, SO₂, NHCO, CHRa and Ra and Rb are independently hydrogen, straight or branched opt. substituted C₁-C₈ alkyl group, aryl C₁-C₆ alkyl group, 5 or 6 membered saturated or unsaturated heterocyclic or heterocyclyl C₁-C₆ alkyl group or aryl; Q represents a resin of general formula Res-B wherein B represents an acid-labile linker, such as, for instance, trityl, (4-methoxyphenyl)(phenyl)-methyl, 4-hydroxyphenyl-methyl, 4-hydroxyphenyl-methyl-oxycarbonyl and the like, while Res represents either a neutral core resin, such as polystyrene resin, or a hydroxyl core resin, such as, for instance, Novagel™ or Tentagel™ resins.

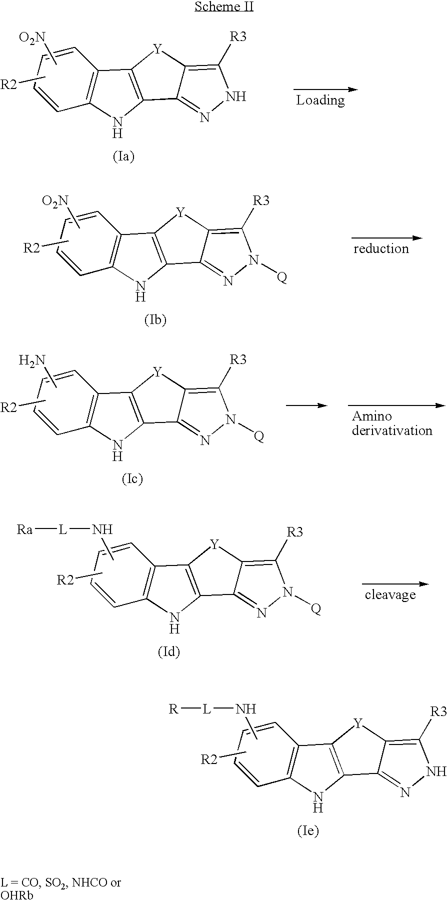

In step one (loading) the tetracyclic derivative is supported on the solid support by reacting it with a resin, for instance, trityl resin, 4-benzyloxybenzyl bromide resin, 4-nitrophenyl carbonate resin and the like using a suitable solvent, like, for instance, dichioromethane, tetrahydrofuran, N,N-dimethylformamide and the like, in the presence of a suitable base, like, for instance, diisopropylethylamine, diazabicyclo[5.4.0]undec-7-ene and the like at temperature ranging from 0 C to about 70 C for a time varying from 15 minutes to 72 hours.

In step two (reduction) the nitro group is reduced by means of well-known methods, such as, for instance, chemical reduction with iron, zinc or tin (II) chloride treatment. The reaction may occur in a suitable solvent such as, for instance, N,N-dimethylformamide, 1,4-dioxane, 1-methyl-2-pyrrolidinone, at a temperature ranging from about −10° C. to reflux and for a suitable time, for instance from about 30 minutes to about 96 hours.

In step three (amino derivativation) acylation of the amino group can be performed by reacting it with carboxylic acids (as formula Xa as defined below) or their derivatives, such as acyl chlorides as formula Xb as defined below) and bromides, with sulphonic acid derivatives, namely sulphonylchlorides (as formula XI as defined below) and bromides, with isocyanates or isothiocyanates (as formula XII as defined below) to yield respectively carboxamido derivatives, sulphonamido derivatives, ureido or thioureido derivatives.

The reaction between the solid-supported tetracyclic derivative and a carboxylic acid can be carried out in the presence of a coupling agent such as, for instance, benzotriazol-1-yloxytris(pyrrolidino)phosphonium hexafluorophosphate, 1,3-dicyclohexylcarbodiimide, 1,3-diisopropylcarbodiimide, o-benzotriazol-1-yl-n,n,n′,n′-tetramethyluronium tetrafluoroborate, carbonyldiimidazole, in a suitable solvent such as, for instance, dichloromethane, chloroform, tetrahydrofuran, 1,4-dioxane, toluene or N,N-dimethylformamide, at a temperature ranging from about −10° C. to the reflux temperature of the solvent and for a suitable time ranging from about 30 minutes to about 96 hours.

The said reaction is optionally carried out in the presence of a suitable catalyst, for instance 4-dimethylaminopyridine, or in the presence of a further coupling agent such as N-hydroxybenzotriazole. The reaction can also be carried out through a mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, 1,4-dioxane or N,N-dimethylformamide, and at a temperature ranging from about −30° C. to room temperature.

The reaction between the solid-supported tetracyclic derivative and an acyl chloride or acyl bromide can be carried out in the presence of a tertiary base such as triethylamine, N,N-diisopropylethylamine or pyridine, in a suitable solvent such as toluene, dichloromethane, chloroform, tetrahydrofuran, or N,N-dimethylformamide, and at a temperature ranging from about −10° C. to the reflux temperature of the solvent.

The reaction between the solid-supported tetracyclic derivative and a sulphonyl derivative, such as the chloride or the bromide, can be carried out in the presence of a tertiary base such as triethylamine, N,N-diisopropylethylamine or pyridine, in a suitable solvent such as toluene, dichloromethane, chloroform, tetrahydrofuran, or N,N-dimethylformamide, at a temperature ranging from about −10° C. to the reflux temperature of the solvent.

Finally, the reaction between the solid-supported tetracyclic derivative and an isocyanate can be carried out in the presence of a tertiary base such as triethylamine, N,N-diisopropylethylamine or pyridine, in a suitable solvent such as toluene, dichloromethane, chloroform, tetrahydrofuran, or N,N-dimethylformamide, and at a temperature ranging from about −10° C. to the reflux temperature of the solvent.

Alternatively the solid-supported tetracyclic derivative is reacted under reductive conditions with an aldehyde (as formula XIII as defined below) or ketone derivative of formula RaRbCO so as to obtain the corresponding amine wherein Ra and Rb are as above defined.

From the above, it is clear to the skilled man that by reacting an aldehyde derivative of RaCORb, for instance wherein Rb is a hydrogen atom, the corresponding derivative wherein L is a —CH₂— group will be obtained; likewise, by reacting a ketone derivative of formula RaCORb, a —CHRb— group will correspond to L.

This reaction, widely known as reductive alkylation of amines, occurs in the presence of a reducing agent such as, for instance, sodium borohydride, sodium cyanoborohydride or sodium triacetoxyborohydride, in a suitable solvent such as N,N-dimethylformamide, N,N-dimethylacetamide, chloroform, dichloromethane, or tetrahydrofuran, optionally in the presence of acetic acid, methanol or ethanol as co-solvents, at a temperature ranging from about 0° C. to reflux and for a time varying from about 30 minutes to about 96 hours.

In step four (cleavage) the final compound of general formula (Ie) is obtained by reacting the compound of general formula (Id) under acidic conditions, for instance, using a certain amount, typically from 1% to 50%, of trifluoroacteic acid in dichloromethane or chloroform at temperature ranging from 0° C. to the reflux temperature of the solvent, for a time ranging from 5 minutes to 10 hours. The synthetic pathway reported in Scheme III illustrates a procedure for conversion into derivatives of general formula (I) containing carboxamides as substituents, where Y, Q, R2 and R3 are as defined above, Rc and Rd are independently hydrogen atom or a suitable organic residue, and R5 is a C₁-C₅ alkyl group, such as, for instance methyl, ethyl and the like.

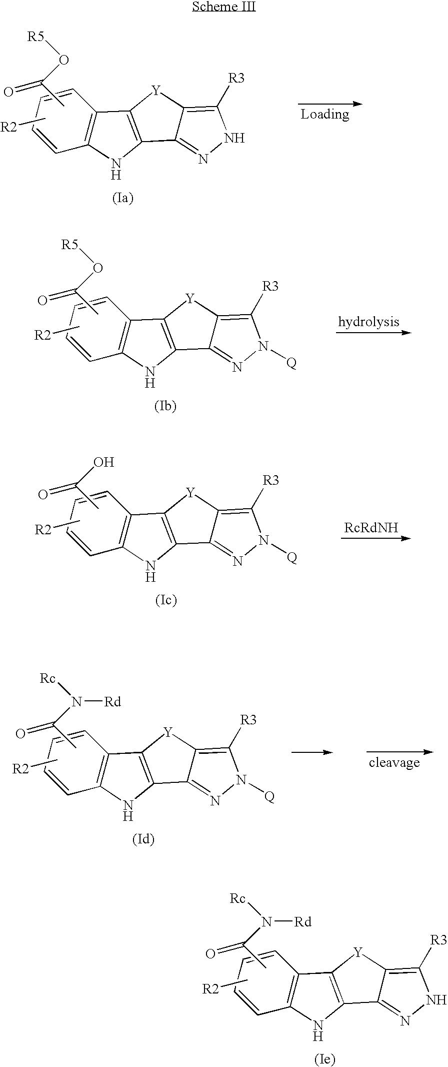

In step one the tetracyclic derivative is supported on the solid support by reacting it with a resin, for instance, trityl resin, 4-benzyloxybenzyl bromide resin, 4-nitrophenyl carbonate resin and the like using a suitable solvent, like, for instance, dichloromethane, tetrahydrofuran, N,N-dimethylformamide and the like, in the presence of a suitable base, like, for instance, diisopropylethylamine, diazabicyclo[5.4.0]undec-7-ene and the like at temperature ranging from 0 C to about 70 C for a time varying from 15 minutes to 72 hours.

In step two the ester is hydrolyzed by using an inorganic base, such as, for instance, lithium hydroxide or sodium hydroxide in a suitable solvent, like tetrahydrofuran, N,N-dimethylformamide, and the like, in the presence of a certain amount of water as a cosolvent, at temperature ranging from 0° C. to the reflux temperature of the solvent, for a time ranging from 1 hour to 96 hours.

In step three the solid-supported carboxyketocycloalkan[b]indole, is reacted with a compound of formula RcRdNH, wherein Rc and Rd are as defined above (as formula XIV as defined below) by means of well known methods. For instance, the reaction can be carried out in the presence of a coupling agent such as, for instance, benzotriazol-1-yloxytris(pyrrolidino)phosphonium hexafluorophosphate, 1,3-dicyclohexylcarbodiimide, 1,3-diisopropylcarbodiimide, o-benzotriazol-1-yl-n,n,n′,n′-tetramethyluronium tetrafluoroborate, carbonyldiimidazole, in a suitable solvent such as, for instance, dichloromethane, chloroform, tetrahydrofuran, 1,4-dioxane, or N,N-dimethylformamide, at a temperature ranging from about −10° C. to the reflux temperature of the solvent and for a suitable time ranging from about 30 minutes to about 96 hours.

The said reaction is optionally carried out in the presence of a suitable catalyst, for instance 4-dimethylamino pyridine, or in the presence of a further coupling agent such as N-hydroxybenzotriazole. The reaction can also be carried out through a mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, 1,4-dioxane or N,N-dimethylformamide, and at a temperature ranging from about −30° C. to room temperature.

In step four the final compound of general formula (Ie′) is obtained by reacting the compound of general formula (Id′) under acidic conditions, for instance, using a certain amount, typically from 1% to 50%, of trifluoroacteic acid in dichloromethane or chloroform at temperature ranging from 0 C to reflux, for a time ranging from 5 minutes to 10 hours.

The synthetic pathway reported in Scheme IV illustrates an alternative procedure for the conversion into compounds of general formula (I) containing carboxamides as substituents, wherein Y, Rc, Rd, R2 and R3 are as defined above, and Qa is a resin of general formula Rs-Ka wherein Ka represents an activated type linker, such as, for instance, 4-hydroxy-2,3,5,6-tetrafluorobenzamide (as described for instance in J. Comb. Chem., 2000, 2, 691), and 4-hydroxy-3-nitrobenzamide (as described for instance in J. Heterocycl. Chem., 2000, 37, 1003) and the like, or a safety catch type linker, such as, for instance, 3-terbutoxy-4-hydroxyaniline (as described for instance in J. Org. Chem., 2001, 66, 2240) and the like. Rs represents a neutral core resin, such as polystyrene resin.

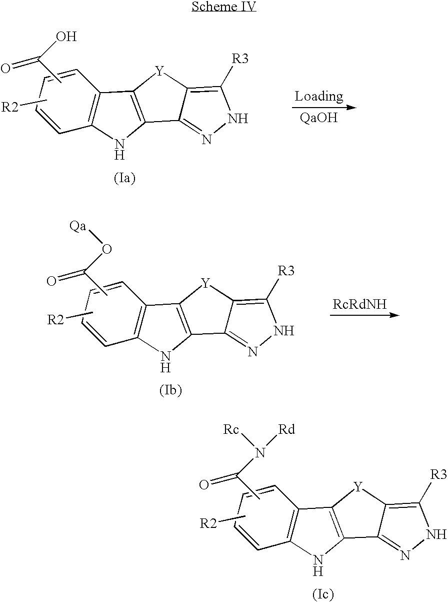

In step one the tetracyclic scaffold, optionally protected at the indole and pyrazole nitrogen atoms with the appropriate protecting groups, is loaded on the resin by means of well known methods, for instance through the mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, 1,4-dioxane or N,N-dimethylformamide, and at a temperature ranging from about −30° C. to room temperature.

Alternatively this reaction can be carried out in the presence of a coupling agent such as, for instance, benzotriazol-1-yloxytris(pyrrolidino)phosphonium hexafluorophosphate, 1,3-dicyclohexylcarbodiimide, 1,3-diisopropylcarbodiimide, o-benzotriazol-1-yl-n,n,n′,n′-tetramethyluronium tetrafluoroborate, carbonyldiimidazole, in a suitable solvent such as, for instance, dichloromethane, chloroform, tetrahydrofuran, 1,4-dioxane, or N,N-dimethylformamide, at a temperature ranging from about −10° C. to reflux and for a suitable time ranging from about 30 minutes to about 96 hours.

The said reaction is optionally carried out in the presence of a suitable catalyst, for instance 4-dimethylaminopyridine, or in the presence of a further coupling agent such as N-hydroxybenzotriazole.

In step two the supported tetracyclic derivative, after activation of the linker when required (as described for instance in J. Org. Chem., 2001, 66, 2240), is treated with a suitable amount of an amine that cleaves the final product of general formula (Ic″) off the resin.

As stated above in general for the conversion of a compound of formula (I) into a different compound of formula (I), converting a compound of general formula (Ia), (Ia′), (Ia″) wherein Y is —CH₂—CH₂— group according to the processes depicted respectively in schemes II, III or IV, may lead to a mixture of a final compound of formula (I) wherein Y is a —CH₂—CH₂— group and a final compound of formula (I) wherein Y is a —CH═CH— group, that is, a fully aromatized compound. The two different compounds of formula (I) can be conveniently separated by known chromatographic technique. It is therefore a further object of the present invention any specific compound of formula (I) which is obtainable through the combinatorial chemistry technique described in scheme II above, by reacting each of the derivatives of formula (X), as set forth in tables I and II, each of the derivatives of formula (XI), as set forth in table III, each of the derivatives of formula (XII), as set forth in table IV, each of the derivatives of formula (XIII), as set forth in table V with any one of the derivatives of formula (Ic), wherein R2, R3 and Q are as defined above, which are obtainable as above indicated.

TABLE I
Compounds of formula (X)
Ra—COOH, (Xa, acid)

| | |
|---|---|
| 1. | 9-fluorenecarboxylic acid; |
| 2. | 1-phenyl-1-cyclopropanecarboxylic acid; |
| 3. | 1-methylcyclopropane-1-carboxylic acid; |
| 4. | Cyclobutanecarboxylic acid; |
| 5. | cyclopentanecarboxylic acid; |
| 6. | (−)-menthoxyacetic acid; |
| 7. | 1,2,3,4-tetrahydro-2-naphthoic acid; |
| 8. | 2-fluorobenzoic acid; |
| 9. | 2,5-dimethoxybenzoic acid; |
| 10. | 2-biphenylcarboxylic acid; |
| 11. | 2-(4-chlorobenzoyl)benzoic acid; |
| 12. | 2,6-dimethylbenzoic acid; |
| 13. | 3-cyanobenzoic acid; |
| 14. | 3-bromobenzoic acid; |
| 15. | 3,4-dimethoxybenzoic acid; |
| 16. | 3,4,5-trimethoxybenzoic acid; |
| 17. | 3,4-diethoxybenzoic acid; |
| 18. | 4-cyanobenzoic acid; |
| 19. | 4-iodobenzoic acid; |
| 20. | 4-diethylaminobenzoic acid; |
| 21. | 4-biphenylcarboxylic acid; |
| 22. | 3-methyl-2-oxovaleric acid; |
| 23. | pyruvic acid; |
| 24. | 2-methylvaleric acid; |
| 25. | tert-butylacetic acid; |
| 26. | 3-(2-methoxyphenyl)propionic acid; |
| 27. | 5-nitro-2-furoic acid; |
| 28. | 1-naphthoic acid; |
| 29. | 2-naphthoic acid; |
| 30. | 2-ketobutyric acid; |
| 31. | pivalic acid; |
| 32. | 2,2-dimethylbutyric acid; |
| 33. | diphenylacetic acid; |
| 34. | N,N-dimethylglycine; |
| 35. | 2,3-dichlorophenoxyacetic acid; |
| 36. | phenylacetic acid; |
| 37. | 2,4-dichlorophenylacetic acid; |
| 38. | 3-fluorophenylacetic acid; |
| 39. | 4-ethoxyphenylacetic acid; |
| 40. | p-tolylacetic acid; |
| 41. | 4-pentynoic acid; |
| 42. | mono-methyl glutarate; |
| 43. | monomethyl adipate; |
| 44. | 6-acetamidohexanoic acid; |
| 45. | 1-pyroglutamic acid; |
| 46. | 3-furoic acid; |
| 47. | thiophene-3-carboxylic acid; |
| 48. | thiophene-3-acetic acid; |
| 49. | nicotinic acid; |
| 50. | nalidixic acid; |
| 51. | 2-nitro-4-trifluoromethylbenzoic acid; |
| 52. | 4-methyl-3-nitrobenzoic acid; |
| 53. | 3-nitrobenzoic acid; |
| 54. | 3-nitrophenylacetic acid; |
| 55. | 4-carboxybenzenesulfonamide; |
| 56. | succinamic acid; |
| 57. | N-(4-nitrobenzoyl)-beta-alanine; |
| 58. | 3-(phenylsulfonyl)propionic acid; |
| 59. | 2,2,3,3-tetramethylcyclopropanecarboxylic |
| | acid; |
| 60. | 2-(4-nitrophenyl)propionic acid; |
| 61. | 2,2-dimethyl-4-pentenoic acid; |
| 62. | 3-(diethylamino)propionicacid hydrochloride; |
| 63. | 4-dimethylaminobutyric acid hydrochloride; |
| 64. | 4-isopropylphenoxyacetic acid; |
| 65. | 5-benzoylpentanoic acid; |
| 66. | 4-acetamido-3-nitrobenzoic acid; |
| 67. | d-campholic acid; |
| 68. | 2,5-dibromobenzoic acid; |
| 69. | 3-acetoxybenzoic acid; |
| 70. | 2,4,6-trimethoxyphenylacetic acid; |
| 71. | 2-benzyloxyphenylacetic acid; |
| 72. | (3,5-dimethoxyphenyl)acetic acid; |
| 73. | 2-nitrophenoxyacetic acid; |
| 74. | chromone-3-carboxylic acid; |
| 75. | N-acetyl-4-fluoro-dl-phenylalanine; |
| 76. | N-m-tolylphthalamic acid; |
| 77. | 4-acetamidobutyric acid; |
| 78. | 3-(2-thenoyl)-propionic acid; |
| 79. | 3,5-diacetamidobenzoic acid; |
| 80. | 5-acetamido-2-nitrobenzoic acid; |
| 81. | acetic acid; |
| 82. | 5-methylhexanoic acid; |
| 83. | N-benzoyl-b-alanine; |
| 84. | 4-bromo-3-methylbenzoic acid; |
| 85. | 4,5-dibromothiophene-2-carboxylic acid; |
| 86. | 2-acetamido-5-bromobenzoic acid; |
| 87. | 4-bromo-2-methylbenzoic acid; |
| 88. | 2-fluoro-6-iodobenzoic acid; |
| 89. | 2-furanglyoxylic acid; |
| 90. | N,N-dimethylsuccinamic acid; |
| 91. | 2-(2-methoxyethoxy)acetic acid; |
| 92. | 4-chloro-alpha-methylphenylacetic acid; |
| 93. | 1-(p-tolyl)-1-cyclopentanecarboxylic acid; |
| 94. | picolinic acid hydrochloride; |
| 95. | 3,5-dibromobenzoic acid; |
| 96. | 5-chlorothianaphthene-3-acetic acid; |
| 97. | 2-nitrothiophene-4-carboxylic acid; |
| 98. | 3-chloro-2-methylbenzoic acid; |
| 99. | 2-bromo-4-fluorobenzoic acid; |
| 100. | 3-(2-chloro-6-fluorophenyl)-5- |
| | methylisoxazole-4-carboxylic acid; |
| 101. | fenbufen; |
| 102. | indoprofen; |
| 103. | chrysanthemum monocarboxylic acid; |
| 104. | 6-acetoxy-2-naphthoic acid; |
| 105. | 3-methylthiopropionic acid; |
| 106. | (R)-(+)-N-(1-phenylethyl)phthalamic acid; |
| 107. | alpha-ketovaleric acid; |
| 108. | 5-methyl-1-phenylpyrazole-4-carboxylic acid; |
| 109. | 3-methyl-1-cyclohexanecarboxylic acid; |
| 110. | 3-methoxycyclohexanecarboxylic acid; |
| 111. | dicyclohexylacetic acid; |
| 112. | 5,6-dichloronicotinic acid; |
| 113. | 4-(dimethylamino)phenylacetic acid; |
| 114. | (R)-(+)-N-(1-phenylethyl)succinamic acid; |
| 115. | (S)-(−)-N-(1-phenylethyl)succinamic acid; |
| 116. | (+)-menthyloxyacetic acid; |
| 117. | suprofen; |
| 118. | N,N-dimethyl-1-phenylalanine; |
| 119. | 4-iodophenylacetic acid; |
| 120. | 4-(3,4-dimethoxyphenyl)butyric acid; |
| 121. | 2-fluoro-5-nitrobenzoic acid; |
| 122. | N,N-diethyl-3,6-difluorophthalamic acid; |
| 123. | 2-bromo-5-nitrobenzoic acid; |
| 124. | 4-bromo-2-fluorobenzoic acid; |
| 125. | 5-(2-thienyl)pentanoic acid; |
| 126. | isoxazole-5-carboxylic acid; |
| 127. | 5-nitrothiophene-2-carboxylic acid; |
| 128. | 2-(4-pyridyl)thiazole-4-carboxylic acid; |
| 129. | 2-methyl-4,4,4-trifluorobutyric acid; |
| 130. | 1-(aminocarbonyl)-1-cyclopropanecarboxylic |
| | acid; |
| 131. | 1-cyanocyclopropanecarboxylic acid; |
| 132. | (S)-(−)-2-acetoxypropionic acid; |
| 133. | 3-(methylsulfonyl)benzoic acid; |
| 134. | 2-chloro-4-methylsulfonylbenzoic acid; |
| 135. | 2,6-dichloropyridine-4-carboxylic acid; |
| 136. | 3-pyridinepropionic acid; |
| 137. | 5-(4-chloro-2-nitrophenyl)-2-furoic acid; |
| 138. | 7-chloro-1-ethyl-6-fluoro-4- |
| | oxohydroquinoline-3-carboxylic acid; |
| 139. | cis-2-(2-thiophenecarbonyl)-1- |
| | cyclohexanecarboxylic acid; |
| 140. | 5-bromo-3-pyridylacetic acid; |
| 141. | 5-methylisoxazole-4-carboxylic acid; |
| 142. | 2,2-dimethylhexanoic acid; |
| 143. | 3-carboxypropanesulfonamide; |
| 144. | 6-cyanonicotinic acid; |
| 145. | (R)-(−)-2-methoxypropionic acid; |
| 146. | (S)-(+)-2-methoxypropionic acid; |
| 147. | 4-(tert-butoxymethyl)benzoic acid; |
| 148. | cis-2-(benzyloxycarbonylamino)- |
| | cyclohexanecarboxylic acid; |
| 149. | cis-2-(benzyloxycarbonylamino)-4- |
| | cyclohexene-1-carboxylic acid. |

TABLE II
Compounds of formula (X)
Ra—COCl, (Xb, acyl chloride)

| | |
|---|---|
| 1. | 3,5-bis(trifluoromethyl)benzoyl chloride |
| 2. | benzoyl chloride |
| 3. | 2-bromobenzoyl chloride |
| 4. | 2-fluorobenzoyl chloride |
| 5. | 2,4-difluorobenzoyl chloride |
| 6. | 2,6-difluorobenzoyl chloride |
| 7. | 2-chlorobenzoyl chloride |
| 8. | 2,4-dichlorobenzoyl chloride |
| 9. | 2-methoxybenzoyl chloride |
| 10. | 2-(trifluoromethyl)benzoyl chloride |
| 11. | o-toluoyl chloride |
| 12. | 3-bromobenzoyl chloride |
| 13. | 3-fluorobenzoyl chloride |
| 14. | 3-chlorobenzoyl chloride |
| 15. | 3,4-dichlorobenzoyl chloride |
| 16. | m-anisoyl chloride |
| 17. | 3,4-dimethoxybenzoyl chloride |
| 18. | 3,4,5-trimethoxybenzoyl chloride |
| 19. | 3,5-dimethoxybenzoyl chloride |
| 20. | 3-(trifluoromethyl)benzoyl chloride |
| 21. | m-toluoyl chloride |
| 22. | 4-bromobenzoyl chloride |
| 23. | 4-fluorobenzoyl chloride |
| 24. | 4-chlorobenzoyl chloride |
| 25. | p-anisoyl chloride |
| 26. | 4-ethoxybenzoyl chloride |
| 27. | 4-n-butoxybenzoyl chloride |
| 28. | 4-biphenylcarbonyl chloride |
| 29. | 4-(trifluoromethyl)benzoyl chloride |
| 30. | 4-tert-butylbenzoyl chloride |
| 31. | p-toluoyl chloride |
| 32. | 4-ethylbenzoyl chloride |
| 33. | 4-n-propylbenzoyl chloride |
| 34. | 4-n-butylbenzoyl chloride |
| 35. | pivaloyl chloride |
| 36. | isobutyryl chloride |
| 37. | 2-ethylhexanoyl chloride |
| 38. | acetyl chloride |
| 39. | phenoxyacetyl chloride |
| 40. | 4-chlorophenoxyacetyl chloride |
| 41. | methoxyacetyl chloride |
| 42. | phenylacetyl chloride |
| 43. | tert-butylacetyl chloride |
| 44. | isovaleryl chloride |
| 45. | propionyl chloride |
| 46. | hydrocinnamoyl chloride |
| 47. | butyryl chloride |
| 48. | pentanoyl chloride |
| 49. | 4-iodobenzoyl chloride |
| 50. | cyclopropanecarbonyl chloride |
| 51. | cyclobutanecarbonyl chloride |
| 52. | cyclopentanecarbonyl chloride |
| 53. | 3-cyclopentylpropionyl chloride |
| 54. | cyclohexanecarbonyl chloride |
| 55. | 4-cyanobenzoyl chloride |
| 56. | 2-furoyl chloride |
| 57. | 1-naphthoyl chloride |
| 58. | 2-naphthoyl chloride |
| 59. | thiophene-2-carbonyl chloride |
| 60. | thiophene-2-acetyl chloride |
| 61. | (3,4-dimethoxyphenyl)acetyl chloride |
| 62. | 3,5-dichlorobenzoyl chloride |
| 63. | 2,5-difluorobenzoyl chloride |
| 64. | 3,4-difluorobenzoyl chloride |
| 65. | 9-fluorenone-4-carbonyl chloride |
| 66. | 3,5-difluorobenzoyl chloride |
| 67. | benzyloxyacetyl chloride |
| 68. | 3-cyanobenzoyl chloride |
| 69. | (2,5-dimethoxyphenyl)acetyl chloride |
| 70. | 3-methoxyphenylacetyl chloride |
| 71. | nicotinoyl chloride hydrochloride |
| 72. | isonicotinoyl chloride hydrochloride |
| 73. | 2,4,6-trimethylbenzoyl chloride |
| 74. | diphenylacetyl chloride |
| 75. | 2-methylvaleryl chloride |
| 76. | 3,4-methylenedioxybenzoyl chloride |
| 77. | 2,4-dimethoxybenzoyl chloride |
| 78. | 2-phenoxypropionyl chloride |
| 79. | 2-phenylbutyryl chloride |
| 80. | 2-ethylbutyryl chloride |
| 81. | 2,3-dichlorobenzoyl chloride |
| 82. | 4-chlorophenylacetyl chloride |
| 83. | dl-2-methylbutyryl chloride |
| 84. | 2,3-difluorobenzoyl chloride |
| 85. | 1-(4-chlorophenyl)-1-cyclopentanecarbonyl- |
| | chloride |
| 86. | 2-ethoxy-1-naphthoyl chloride |
| 87. | benzo[b]thiophene-2-carbonyl chloride |
| 88. | 4-(trifluoromethoxy)benzoyl chloride |
| 89. | 2-(trifluoromethoxy)benzoyl chloride |
| 90. | 3-chlorobenzo[b]thiophene-2-carbonyl |
| | chloride |
| 91. | 2-fluoro-3-(trifluoromethyl)benzoyl chloride |
| 92. | 2-fluoro-4-(trifluoromethyl)benzoyl chloride |
| 93. | 2-fluoro-5-(trifluoromethyl)benzoyl chloride |
| 94. | 3-fluoro-5-(trifluoromethyl)benzoyl chloride |
| 95. | 4-fluoro-2-(trifluoromethyl)benzoyl chloride |
| 96. | 4-fluoro-3-(trifluoromethyl)benzoyl chloride |
| 97. | 2-fluoro-6-(trifluoromethyl)benzoyl chloride |
| 98. | 2,3,6-trifluorobenzoyl chloride |
| 99. | 2,4,5-trifluorobenzoyl chloride |
| 100. | 3-(trifluoromethoxy)benzoyl chloride |
| 101. | isoxazole-5-carbonyl chloride |
| 102. | 2,4,6-trifluorobenzoyl chloride |
| 103. | 2,5-bis(trifluoromethyl)benzoyl chloride |
| 104. | 2,3,4-trifluorobenzoyl chloride |
| 105. | 2,4,6-trichlorobenzoyl chloride |
| 106. | 2,4-dichloro-5-fluorobenzoyl chloride |
| 107. | 4-methoxyphenylacetyl chloride |
| 108. | 5-fluoro-2-(trifluoromethyl)benzoyl chloride |
| 109. | 2-chloro-6-fluorobenzoyl chloride |
| 110. | 2-bromo-5-methoxybenzoyl chloride |
| 111. | cyclopentylacetyl chloride |
| 112. | 3-chloro-4-fluorobenzoyl chloride |
| 113. | 3-fluoro-4-(trifluoromethyl)benzoyl chloride |
| 114. | 4-fluorophenylacetyl chloride |
| 115. | 4-tert-butylphenoxyacetyl chloride |
| 116. | 7-Imidazol-1-yl-5,6-dihydro-naphthalene-2- |
| | carbonyl chloride |
| 117. | 4-Imidazol-1-ylmethyl-benzoyl chloride |
| 118. | 4-bromo-3-methylbenzoyl chloride |

TABLE III
Compounds of formula (XI)
Sulfonyl chloride of formula Ra—SO₂Cl (XI)

| | |
|---|---|
| 1. | 1-naphthalenesulfonyl chloride |
| 2. | 2-naphthalenesulfonyl chloride |
| 3. | 2-thiophenesulfonyl chloride |
| 4. | 8-quinolinesulfonyl chloride |
| 5. | benzenesulfonyl chloride |
| 6. | 2,4,5-trichlorobenzenesulfonyl chloride |
| 7. | 2,5-dichlorobenzenesulfonyl chloride |
| 8. | 3,5-dichloro-2-hydroxybenzenesulfonyl chloride |
| 9. | 2-mesitylenesulfonyl chloride |
| 10. | 4-bromobenzenesulfonyl chloride |
| 11. | 4-fluorobenzenesulfonyl chloride |
| 12. | 4-chlorobenzenesulfonyl chloride |
| 13. | pipsyl chloride |
| 14. | 4-methoxybenzenesulfonyl chloride |
| 15. | 4-tert-butylbenzenesulfonyl chloride |
| 16. | p-toluenesulfonyl chloride |
| 17. | isopropylsulfonyl chloride |
| 18. | methanesulfonyl chloride |
| 19. | alpha-toluenesulfonyl chloride |
| 20. | ethanesulfonyl chloride |
| 21. | 1-propanesulfonyl chloride |
| 22. | 1-butanesulfonyl chloride |
| 23. | Pentamethylbenzenesulfonyl chloride |
| 24. | 2,3,5,6-tetramethylbenzenesulfonyl chloride |
| 25. | 3-(trifluoromethyl)benzenesulphonyl chloride |
| 26. | 3,5-bis(trifluoromethyl)benzenesulfonyl chloride |
| 27. | 2,3,4-trichlorobenzenesulfonyl chloride |
| 28. | 2,5-dimethoxybenzenesulfonyl chloride |
| 29. | 4-methoxy-2,3,6-trimethylbenzenesulfonyl |
| | chloride |
| 30. | 3,4-dichlorobenzenesulfonyl chloride |
| 31. | 4,5-dibromothiophene-2-sulfonyl chloride |
| 32. | 3-chloro-4-fluorobenzenesulphonyl chloride |
| 33. | 4-ethylbenzenesulfonyl chloride |
| 34. | 4-N-propylbenzenesulfonyl chloride |
| 35. | 4-N-amylbenzenesulfonyl chloride |
| 36. | 4-isopropylbenzenesulphonyl chloride |
| 37. | 4-bromo-2,5-difluorobenzenesulphonyl chloride |
| 38. | 2-fluorobenzenesulphonyl chloride |
| 39. | 3-fluorobenzenesulphonyl chloride |
| 40. | 4-(trifluoromethoxy)benzenesulphonyl chloride |
| 41. | 4-(trifluoromethyl)benzenesulfonyl chloride |
| 42. | 2,4-difluorobenzenesulphonyl chloride |
| 43. | 2,4-dichloro-5-methylbenzenesulfonyl chloride |
| 44. | 4-chloro-2,5-dimethylbenzenesulphonyl chloride |
| 45. | 2-chlorobenzenesulfonyl chloride |
| 46. | 4-bromo-2,5-dichlorothiophene-3-sulfonyl |
| | chloride |
| 47. | 2,5-dichlorothiophene-3-sulphonyl chloride |
| 48. | 5-chlorothiophene-2-sulfonyl chloride |
| 49. | 2-(trifluoromethyl)benzenesulfonyl chloride |
| 50. | 3-chlorobenzenesulfonyl chloride |
| 51. | 3,5-dichlorobenzenesulfonyl chloride |
| 52. | m-toluenesulfonyl chloride |
| 53. | 2-chloro-6-methylbenzenesulfonyl chloride |
| 54. | 5-bromo-2-methoxybenzenesulfonyl chloride |
| 55. | 3,4-dimethoxybenzenesulfonyl chloride |
| 56. | 2,3-dichlorobenzenesulfonyl chloride |
| 57. | 2-bromobenzenesulfonyl chloride |
| 58. | 2,3-dichlorothiophene-5-sulphonyl chloride |
| 59. | 4-phenylthiophene-2,4-disulfonyl |
| 60. | 5-phenylthiophene-2,5-disulfonyl chloride |
| 61. | 3-chloro-2-methylbenzenesulfonyl chloride |
| 62. | 2-chloro-5-(trifluoromethyl)benzenesulfonyl |
| | chloride |
| 63. | 2,6-dichlorobenzenesulfonyl chloride |
| 64. | 3-bromobenzenesulfonyl chloride |
| 65. | 2-(trifluoromethoxy)benzenesulfonyl chloride |
| 66. | 4-cyanobenzenesulfonyl chloride |
| 67. | 2-cyanobenzenesulfonyl chloride |
| 68. | 4-(N-butoxy)benzenesulfonyl chloride |
| 69. | 4-acetamido-3-chlorobenzenesulfonyl chloride |
| 70. | 3,5-dimethylisoxazole-4-sulfonyl chloride |
| 71. | 2,4-dichlorobenzenesulfonyl chloride |
| 72. | 2-chloro-4-fluorobenzenesulphonyl chloride |
| 73. | 5-fluoro-2-methylbenzenesulphonyl chloride |
| 74. | 5-chloro-2-methoxybenzenesulfonyl chloride |
| 75. | 2,4,6-trichlorobenzenesulfonyl chloride |
| 76. | 4-biphenylsulfonyl chloride |
| 77. | 5-bromothiophene-2-sulfonyl chloride |
| 78. | 2,6-difluorobenzenesulfonyl chloride |
| 79. | 4-n-butylbenzenesulfonyl chloride |
| 80. | 4-methylsulfonylbenzenesulfonyl chloride |
| 81. | 2-methylsulfonylbenzenesulfonyl chloride |
| 82. | 4-acetylbenzenesulfonyl chloride |
| 83. | 3-methoxybenzenesulphonyl chloride |
| 84. | 2-methoxy-4-methylbenzenesulphonyl chloride |

TABLE IV
Compounds of formula (XII)
Isocyanate derivatives of formula (XII, Ra—NCO, Ra—NCS)

| | |
|---|---|
| 1. | Phenyl isocyanate |
| 2. | 2-bromophenyl isocyanate |
| 3. | 2-fluorophenyl isocyanate |
| 4. | 2,4-difluorophenyl isocyanate |
| 5. | 2,6-difluorophenyl isocyanate |
| 6. | 2-chlorophenyl isocyanate |
| 7. | 2,3-dichlorophenyl isocyanate |
| 8. | 2,4-dichlorophenyl isocyanate |
| 9. | 2,5-dichlorophenyl isocyanate |
| 10. | 2,6-dichlorophenyl isocyanate |
| 11. | 2-methoxyphenyl isocyanate |
| 12. | 2,4-dimethoxyphenyl isocyanate |
| 13. | 2,5-dimethoxyphenyl isocyanate |
| 14. | 2-ethoxyphenyl isocyanate |
| 15. | 2-(trifluoromethyl)phenyl isocyanate |
| 16. | o-tolyl isocyanate |
| 17. | 2,6-dimethylphenyl isocyanate |
| 18. | 2-ethylphenyl isocyanate |
| 19. | 3-bromophenyl isocyanate |
| 20. | 3-fluorophenyl isocyanate |
| 21. | 3-chlorophenyl isocyanate |
| 22. | 3,4-dichlorophenyl isocyanate |
| 23. | 3-methoxyphenyl isocyanate |
| 24. | 3-(trifluoromethyl)phenyl isocyanate |
| 25. | m-tolyl isocyanate |
| 26. | 4-bromophenyl isocyanate |
| 27. | 4-fluorophenyl isocyanate |
| 28. | 4-chlorophenyl isocyanate |
| 29. | 4-methoxyphenyl isocyanate |
| 30. | 4-(trifluoromethyl)phenyl isocyanate |
| 31. | p-tolyl isocyanate |
| 32. | benzoyl isocyanate |
| 33. | 1-naphthyl isocyanate |
| 34. | Benzyl isocyanate |
| 35. | 3,5-bis(trifluoromethyl)phenyl isocyanate |
| 36. | 2,5-difluorophenyl isocyanate |
| 37. | 2,4,5-trichlorophenyl isocyanate |
| 38. | 2,4,6-trichlorophenyl isocyanate |
| 39. | 2-isopropylphenyl isocyanate |
| 40. | 2,3-dimethylphenyl isocyanate |
| 41. | 4-methoxy-2-methylphenyl isocyanate |
| 42. | 2,4-dimethylphenyl isocyanate |
| 43. | 2,5-dimethylphenyl isocyanate |
| 44. | 2-ethyl-6-methylphenyl isocyanate |
| 45. | 3-cyanophenyl isocyanate |
| 46. | 5-chloro-2,4-dimethoxyphenyl isocyanate |
| 47. | 3-chloro-4-methylphenyl isocyanate |
| 48. | 3,5-dichlorophenyl isocyanate |
| 49. | 5-chloro-2-methoxyphenyl isocyanate |
| 50. | 3,4,5-trimethoxyphenyl isocyanate |
| 51. | 3,5-dimethoxyphenyl isocyanate |
| 52. | 3-(methylthio)phenyl isocyanate |
| 53. | 3-acetylphenyl isocyanate |
| 54. | 3,4-dimethylphenyl isocyanate |
| 55. | 3,5-dimethylphenyl isocyanate |
| 56. | 2-methoxy-5-methylphenyl isocyanate |
| 57. | 3-ethylphenyl isocyanate |
| 58. | 4-bromo-2-(trifluoromethyl)phenyl isocyanate |
| 59. | 4-chloro-2-(trifluoromethyl)phenyl isocyanate |
| 60. | 4-chloro-3-(trifluoromethyl)phenyl isocyanate |
| 61. | 4-iodophenyl isocyanate |
| 62. | 4-phenoxyphenyl isocyanate |
| 63. | 4-ethoxyphenyl isocyanate |
| 64. | 4-acetylphenyl isocyanate |
| 65. | 4-isopropylphenyl isocyanate |
| 66. | 4-ethylphenyl isocyanate |
| 67. | 4-n-butylphenyl isocyanate |
| 68. | 2,4,6-trimethylphenyl isocyanate |
| 69. | 2-isopropyl-6-methylphenyl isocyanate |
| 70. | 2,6-diethylphenyl isocyanate |
| 71. | 5-chloro-2-methylphenyl isocyanate |
| 72. | 4-chloro-2-methylphenyl isocyanate |
| 73. | 4-(trifluoromethoxy)phenyl isocyanate |
| 74. | 2-chloro-5-(trifluoromethyl)phenyl isocyanate |
| 75. | 2-chloro-6-methylphenyl isocyanate |
| 76. | 2,4,5-trimethylphenyl isocyanate |
| 77. | 3-chloro-2-methoxyphenyl isocyanate |
| 78. | 3-chloro-2-methylphenyl isocyanate |
| 79. | 3-chloro-4-fluorophenyl isocyanate |
| 80. | 4-bromo-2-methylphenyl isocyanate |
| 81. | 4-bromo-2,6-dimethylphenyl isocyanate |
| 82. | 2,6-dibromo-4-fluorophenyl isocyanate |
| 83. | 4-butoxyphenyl isocyanate |
| 84. | 3-fluoro-4-methylphenyl isocyanate |
| 85. | 5-fluoro-2-methylphenyl isocyanate |
| 86. | 2-biphenylyl isocyanate |
| 87. | 4-biphenylyl isocyanate |
| 88. | 2-bromo-4,6-difluorophenyl isocyanate |
| 89. | (r)-(+)-1-phenylethyl isocyanate |
| 90. | 1-(1-naphthyl)ethyl isocyanate |
| 91. | (s)-(+)-1-(1-naphthyl) ethyl isocyanate |
| 92. | 3,4-difluorophenyl isocyanate |
| 93. | 2-(trifluoromethoxy)phenyl isocyanate |
| 94. | 4-benzyloxyphenyl isocyanate |
| 95. | 4-bromo-2-chlorophenyl isocyanate |
| 96. | 4-bromo-2-fluorophenyl isocyanate |
| 97. | 2-fluoro-5-methylphenyl isocyanate |
| 98. | 2,3,4-trifluorophenyl isocyanate |
| 99. | 2-(difluoromethoxy)phenyl isocyanate |
| 100. | 4-(difluoromethoxy)phenyl isocyanate |
| 101. | 2-methylbenzyl isocyanate |
| 102. | 2-chlorobenzyl isocyanate |
| 103. | 4-fluorobenzyl isocyanate |
| 104. | 4-methoxybenzyl isocyanate |
| 105. | 2,6-difluorobenzoyl isocyanate |
| 106. | 4-fluorobenzoyl isocyanate |
| 107. | 2-fluoro-3-(trifluoromethyl)phenyl isocyanate |
| 108. | 2-fluoro-5-(trifluoromethyl)phenyl isocyanate |
| 109. | 2-fluoro-6-(trifluoromethyl)phenyl isocyanate |
| 110. | 4-fluoro-2-(trifluoromethyl)phenyl isocyanate |
| 111. | 2-(tert-butyl)phenyl isocyanate |
| 112. | 3-pyridyl isocyanate |

TABLE V
Compounds of formula (XIII)
Aldehyde derivatives of formula (XIII) Ra—COH

| | |
|---|---|
| 1. | 3,5-diiodo-4-hydroxybenzaldehyde |
| 2. | 3-iodobenzaldehyde |
| 3. | 3,5-dibromobenzaldehyde |
| 4. | 4-bromothiophene-2-carboxaldehyde |
| 5. | 2-naphthaldehyde |
| 6. | n-ethyl-carbazole-3-aldehyde |
| 7. | 4-chloro-1-methylpyrazole-3-carboxaldehyde |
| 8. | (3-formyl-l-phenyl-1h-pyrazol-5-yl)methyl |
| | acetate |
| 9. | 1-acetyl-3-indolecarboxaldehyde |
| 10. | methyl 4-formyl-1-methylpyrrole-2-carboxylate |
| 11. | 3,5-di-tert-butyl-4-hydroxybenzaldehyde |
| 12. | 5-(methylthio)-2-thiophenecarboxaldehyde |
| 13. | 4-(methylthio)benzaldehyde |
| 14. | 3-nitro-4-(2-pyridylthio)benzaldehyde |
| 15. | 5-methyl-2-thiophenecarboxaldehyde |
| 16. | 3-acetoxybenzaldehyde |
| 17. | 3,4-dimethylbenzaldehyde |
| 18. | 4-pyridinecarboxaldehyde n-oxide |
| 19. | 4-fluoro-3-methylbenzaldehyde |
| 20. | 2,6-dichloroisonicotinaldehyde |
| 21. | 5-(2,4-difluorophenyl)-2-furaldehyde |
| 22. | 2-(4-bromobenzoyl)-1-benzofuran-5-carbaldehyde |
| 23. | 2-benzoyl-1-benzofuran-5-carbaldehyde |
| 24. | 2-butyl-4-formylimidazole |
| 25. | 5-benzyloxy-1h-pyrrolo[2,3-c]pyridine-3- |
| | carboxaldehyde |
| 26. | 6-methyl-2-pyridinecarboxaldehyde |
| 27. | 4-[4-(tert-butyl)thiazol-2-yl]benzaldehyde |
| 28. | 5-formyl-2,4-dimethoxy-pyrimidine |
| 29. | 2-[(4-chlorobenzyl)thio]pyrimidine-4- |
| | carbaldehyde |
| 30. | 3-fluoro-2-hydroxybenzaldehyde |
| 31. | 3-hydroxybenzaldehyde |
| 32. | 3-carboxybenzaldehyde |
| 33. | 4-vinylbenzaldehyde |
| 34. | 5-(2,5-dichlorophenyl)-2-furaldehyde |
| 35. | 2-fluoro-5-nitrobenzaldehyde |
| 36. | 5-(4-nitrophenyl)-2-furaldehyde |
| 37. | 4-dimethylaminobenzaldehyde |
| 38. | 4-[3-(dimethylamino)propoxy]benzaldehyde |
| 39. | 4-n-butylbenzaldehyde |
| 40. | 4-(4-benzylpiperazino)benzaldehyde |
| 41. | 2,2′-bithiophene-5-carboxaldehyde |
| 42. | 4-[4-(1-adamantyl)-1,3-thiazol-2-yl]benzaldehyde |
| 43. | 4-formyl-trans-stilbene |
| 44. | 6-chloroimidazo[2,1-b][1,3]thiazole-5- |
| | carbaldehyde |
| 45. | 4-(phenylethynyl)benzaldehyde |
| 46. | 3,3′-(4-formylphenylimino)dipropionitrile |
| 47. | 6-formyl-2-(methylthio)nicotinonitrile |
| 48. | 4-cyanobenzaldehyde |
| 49. | 3-[(4-formylphenoxy)methyl]thiophene-2- |
| | carbonitrile |
| 50. | 2-(3-formyl-1h-indol-1-yl)benzonitrile |
| 51. | 2-formyl-6-methoxyphenyl 2,6-difluorobenzoate |
| 52. | tert-butyl 4-formyl-2-methoxyphenyl carbonate |
| 53. | 4-(difluoromethoxy)benzaldehyde |
| 54. | 2-[1-methyl-5-(trifluoromethyl)pyrazol-3- |
| | yl]thiophene-5-carboxaldehyde |
| 55. | 5-(3-trifluoromethylphenyl)furan-2- |
| | carboxaldehyde |
| 56. | 2,3-difluoro-4-methylbenzaldehyde |
| 57. | 3-chloro-5-(trifluoromethyl)pyridine-2- |
| | carboxaldehyde |
| 58. | 4-(trifluoromethoxy)benzaldehyde |
| 59. | 3-[(2,4-difluorophenyl)thio]-5- |
| | (trifluoromethyl)pyridine-2-carbaldehyde |
| 60. | 3,5-bis(trifluoromethyl)benzaldehyde |
| 61. | 2,3,5,6-tetrafluorobenzaldehyde |
| 62. | 4-(methylsulfonyl)benzaldehyde |
| 63. | 1-[(4-methylphenyl)sulfonyl]-1h-indole-3- |
| | carbaldehyde |
| 64. | 4-formyl-2-methoxyphenyl 2,4,5- |
| | trichlorobenzenesulfonate |
| 65. | 4-formylphenyl 2,3,4,5,6- |
| | pentamethylbenzenesulfonate |
| 66. | 3-(4-formylphenyl)-2-(pyridin-2- |
| | ylsulfonyl)acrylonitrile |
| 67. | 4-acetamidobenzaldehyde |
| 68. | 4-[[5-chloro-2-oxopyrimidin-1(2h)- |
| | yl]methoxy]benzaldehyde |
| 69. | 4-(5-formyl-2-furyl)benzene-1-sulfonamide |
| 70. | 3-Benzo[1,3]dioxol-5-yl-2-methyl-propionaldehyde |
| 71. | 3-(phenylthio)butyraldehyde |
| 72. | 3-chloro-4,4,4-trifluoro-2-phenylbutanal |
| 73. | 2-cyano-2-phenylacetaldehyde |
| 74. | 3-methoxyphenylacetaldehyde |
| 75. | pyridine-3-carboxaldehide |
| 76. | 4-chlorobenzaldehyde |
| 77. | 4-cyanobenzaldehyde |
| 78. | 3-fluorobenzaldehyde |
| 79. | m-tolualdehyde |
| 80. | 2,4-dichlorobenzaldehyde |
| 81. | quinoline-3-carboxaldehyde |
| 82. | 2-(trifluoromethyl)benzaldehyde |
| 83. | methyl 4-formylbenzoate |
| 84. | 4-chloro-3-fluorobenzaldehyde |
| 85. | 4-nitrocinnamaldehyde |
| 86. | 3-thiophenecarboxaldehyde |
| 87. | 3-methoxybenzaldehyde |
| 88. | propionaldehyde |
| 89. | 3,3-dimethylbutyraldehyde |
| 90. | 3-phenylpropionaldehyde |

It is a further object of the present invention any specific compound of formula (I) which is obtainable through the combinatorial chemistry technique described in scheme III, by reacting each of the derivatives of formula (XIV), as set forth in tables VI, with any one of the derivatives of formula (Ic′) of scheme III, wherein Y, Rc, Rd, R2, R3 and Q are as defined above, which are obtainable as above indicated.

TABLE VI
Compounds of formula (XIV)
Amine derivatives of formula (XIV) RcRdNH

| | |
|---|---|
| 1. | piperidine |
| 2. | butylamine |
| 3. | 4-(2-aminoethyl)morpholine |
| 4. | 1-(3-aminopropyl)imidazole |
| 5. | piperazine |
| 6. | tetrahydrofurfurylamine |
| 7. | phenethylamine |
| 8. | 3-phenylpropylamine |
| 9. | n-propylamine |
| 10. | isobutylamine |
| 11. | cyclopropanemethylamine |
| 12. | 2-(2-aminoethyl)-1-methylpyrolidine |
| 13. | 4-methylpiperidine |
| 14. | 1-methylpiperazine |
| 15. | 1-(3-aminopropyl)-2-pyrrolidinone |
| 16. | 1,3-diaminopropane |
| 17. | ethylenediamine |
| 18. | 4-hydroxypiperidine |
| 19. | 3-amino-1-propanol |
| 20. | 2-(2-aminoethyl)pyridine |
| 21. | 1-(2-aminoethyl)piperidine |
| 22. | pyrrolidine |
| 23. | n-acetylethylenediamine |
| 24. | 1-acetylpiperazine |
| 25. | 3-methoxypropylamine |
| 26. | 3-methylpiperidine |
| 27. | 2-methylbutylamine |
| 28. | 1-(2-pyridyl)piperazine |
| 29. | 4-benzylpiperidine |
| 30. | n,n-diethylnipecotamide |
| 31. | 3,5-dimethylpiperidine |
| 32. | 2-(aminomethyl)-1-ethylpyrrolidine |
| 33. | 1-(2-furoyl)piperazine |
| 34. | thiophene-2-ethylamine |
| 35. | 1-(2-aminoethyl)-2-imidazolone |
| 36. | thiomorpholine |
| 37. | propargyl chloroformate |
| 38. | 4-piperidinopiperidine |
| 39. | 1-piperazinecarboxaldehyde |
| 40. | 1-benzylpiperazine |
| 41. | 3-piperidinemethanol |
| 42. | 3-ethoxypropylamine |
| 43. | isoamylamine |
| 44. | 1-(2-fluorophenyl)piperazine |
| 45. | 1-(2-hydroxyethyl)-piperazine |
| 46. | n,n-diethylethylenediamine |
| 47. | 1-(2-methoxyphenyl)piperazine |
| 48. | 4-(1-pyrrolidinyl)piperidine |
| 49. | 3-(dimethylamino)propylamine |
| 50. | 2-phenyl-propylamine |
| 51. | 3-hydroxypiperidine |
| 52. | 1-(3 aminopropyl) pyrrolidene |
| 53. | 1-hydroxyethylethoxypiperazine |
| 54. | 2,6-dimethylpiperazine |
| 55. | 3-isopropoxypropylamine |
| 56. | 1-(2,3-dimethylphenyl)-piperazine |
| 57. | 1-(3-methoxyphenyl)-piperazine |
| 58. | n,n-diisopropylethylenediamine |
| 59. | (x)-(−)-2-methylpiperazine |
| 60. | 1-(2,5-dimethylphenyl)piperazine |
| 61. | 2-methyl-1-(3-methylphenyl)piperazine |
| 62. | 1-cyclohexylpiperazine |
| 63. | 2-methylpiperazine |
| 64. | 1-(4-fluorophenyl)piperazine |
| 65. | 1-ethylpropylamine |
| 66. | dl-alpha-methylbenzylamine |
| 67. | 3,4-dimethoxybenzylamine [veratrylamine] |
| 68. | 2-methylbenzylamine |
| 69. | 2-methoxyethylamine |
| 70. | allylamine |
| 71. | azetidine hydrochloride |
| 72. | Ammonia |

It is a further object of the present invention any specific compound of formula (I) which is obtainable through the combinatorial chemistry technique described in scheme IV, by reacting each of the derivatives of formula (XIV), as set forth in tables VI, with any one of the derivatives of formula (Ib″), wherein Y, Rc, Rd, R2, R3 and Qa are as defined above, which are obtainable as above indicated.

As it will be really appreciated by the man skilled in the art, when preparing the compounds of formula (I) object of the invention, optional functional groups within both the starting materials or the intermediates thereof which could give rise to unwanted side reactions, need to be properly protected according to conventional techniques. Likewise, the conversion of these latter into the free deprotected compounds may be carried out according to known procedures.

In addition to the above, it is also clear to the skilled man that the compounds of formula (I) of the invention can be advantageously prepared by combining the above described reactions in a combinatorial fashion, for example according to solid-phase-synthesis (SPS) techniques, so as to get a combinatorial chemical library of compounds of formula (I). It is therefore a further object of the invention a library of two or more compounds of formula (I) as defined above, which can be obtained starting from one or more compound supported onto a solid support of the formula (Ic), (Ic′) or (Ib″) as defined above.

It is a further object of the present invention to provide useful intermediates of formula VII and VIIa as defined above, preferably those of formula VII wherein Y is a —CH₂—CH₂— group, with the proviso that when R2 is a hydrogen atom, and

i) W is dimethylamino and Z is a hydrogen atom, then R1 is not 7-chloro, hydrogen, 7-bromo atom, 7-cyclohexyl or 7-methyl group, or

ii) W is hydroxy and Z is a hydrogen atom, then R1 is not hydrogen, 7-methoxy group, 7-benzyloxy, or

iii) W is hydroxy and Z is ethyloxycarbonyl group, then R1 is not hydrogen.

For clarity, we point out that the framework of the preferred compounds of formula (VII) of the present invention is numbered as follows:

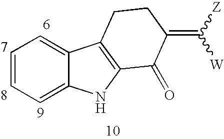

The present invention also provides a process for preparing a compound of the formula (VII) or (VIIa) as above defined, which process comprises:

either i) reacting a compound of formula (VI):

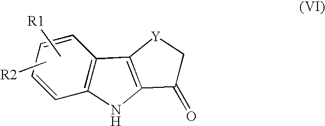

wherein Y, R1 and R2 are as above defined and the indole nitrogen is optionally protected with an appropriate protecting group, with any of the following:
a) a dialkylacetale of dimethylformammide;
b) a carboxylic ester such as alkyl formate, alkyl oxalate, alkyl acetate and the like;
c) dimethyl trithiocarbonate and an alkyl iodide or bromide such as, for instance, methyl iodide or benzyl bromide,
to give a compound of general formula (VII) wherein Y is —(CH₂)ₙ—; n, R1 and R2 are as above defined; W and Z have, respectively, one of the following couple of meanings:
a) W is a dialkylamino group, and Z is a hydrogen atom;
b) W is a hydroxy group, and Z is a hydrogen atom, a C₁-C₄ alkoxycarbonyl group or a methyl group;
c) Z is a C₁-C₆ alkylthio or arylC₁-C₆ alkylthio group, and W is a methylthio group;

and iia) optionally reacting a compound of general formula (VII) where R1, R2 and Y are as described above and W and Z are as defined under c) with any of the following:
a′) an aliphatic or aromatic primary or secondary amine;
b′) a compound of general formula W(CH₂)X where W and X are, the same or different, electron withdrawing groups, such as, for instance, nitrile, alkoxycarbonyl, aryl including heteroaryl groups;
c′) an organometallic compound of general formula RM, where R is either an aliphatic or aromatic group, and M represents lithium or magnesium halide;
d′) an organometallic compound of general formula of (CH₃)₂CuLi₂B, where B is a suitable anion species, like, for instance, a cyano group;
e′) an inorganic cyanide, such as, for instance, sodium cyanide, copper cyanide;
to give a different compound of general formula (VII) where R1, R2 and Y are as defined above, while Z is a C₁-C₆ alkylthio or aryl C₁-C₆ alkylthio group, for instance a methylthio or a benzylthio group and W is
i) a substituted or disubstituted amino group, such as an alkylamino or arylamino group;
ii) a group of general formula —CH(J) (X) where J and X are, the same or different, electron withdrawing groups, such as, for instance, nitrile, alkoxycarbonyl, aryl including heteroaryl groups;
iii) an alkyl or aryl group;
iv) an alkyl- or aryl-carbonyl group;
v) a cyano group or
d)both Z and W are substituted or disubstituted amino groups,

or ii) reacting another compound of formula (VIa):

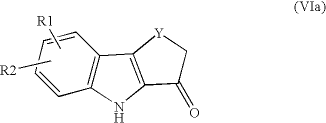

wherein Y is —(CH₂)₂—, R1 and R2 are as above defined, with POCl₃ in dimethylformamide, to give a compound of general formula (VIIa) as defined above.

The reaction i) with the reagents under b) may be carried out in the presence of a strong base like sodium hydride or potassium hydride or sodium methoxyde in solvents like dimethylformamide, tetrahydrofuran and the like, as described for instance Pharmaceut. Chem. J. 1994, 28, 566; JCS Perkin1 1979, 1706; J. Chem. Res. Synop. 1995, 350.

The reaction i) with the reagents under c) may be carried out in the presence of a strong base like potassium terbutoxide, sodium hydride, lithium bis(trimethylsilyl)amide, in solvents like tetrahydrofuran, dimethylformamide and the like, using temperature ranging from −78° to 100° C. The optional reaction iia) with the reagents under b′) may be carried out in the presence of a suitable base, for instance sodium hydride in a inert solvent like tetrahydrofuran or dimethylformamide, at temperature ranging from 0° C. to 100° C.

The optional reaction iia) with the reagents under c′) may be carried out in the optional presence of a certain amount a transition metal-based salt or complex, such as, for instance, copper (I) iodide, copper (I) bromide, copper (I) chloride, in a inert solvent like tetrahydrofuran or dimethoxyethane at temperature ranging from −20° C. to 100° C.

The optional reaction iia) with the reagents under d′) may be carried out in the presence of an aliphatic or aromatic acyl chloride, as described for example in Chem. Lett. 1994, 437.

The processes for preparing the compounds of the formula (VI) as defined above can be conveniently described as set forth below according to the following scheme:

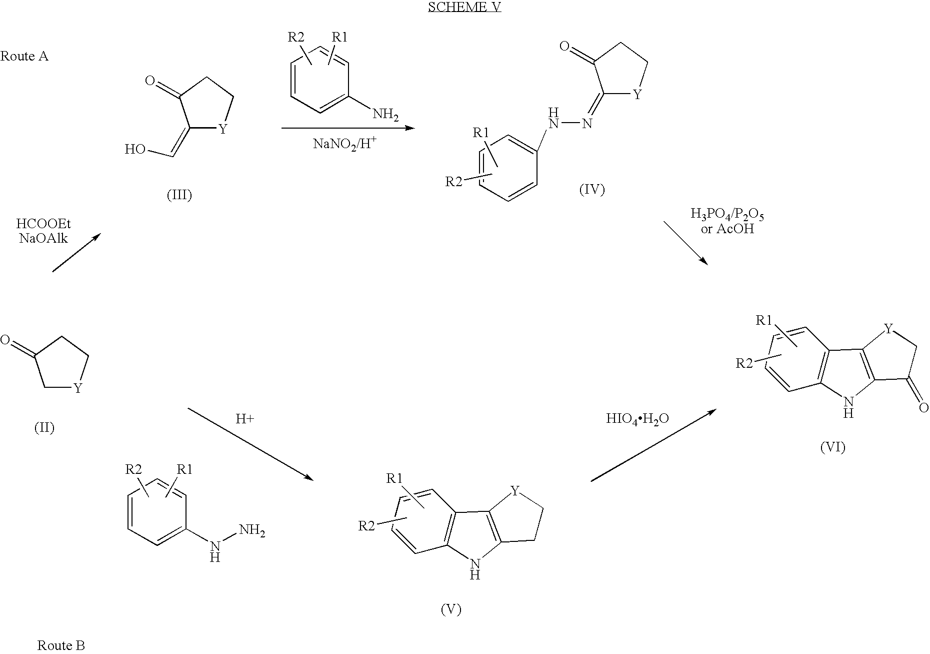

Scheme V describes the synthesis of the ketocycloalkan[b]indole of general formula (VI), where R1, R2 and Y are as described above, which represent key intermediates in the synthesis of the compounds object of the present invention. In route A step one, a cycloalkanone derivative is formylated with ethylformate in the presence of a base like sodium alkoxyde in an inert solvent like, for instance diethyl ether, as described in Organic Syntheses 1963, vol. 4, 536. In step two the aryldiazonium salt, prepared from the aniline and sodium nitrite in acids, is added to a basic hydro alcoholic solution of the cycloalkanone derivative to yield the corresponding hydrazone, as described for instance in Chem. Pharm. Bull. 1981, 699.

Step three describes the Fischer indolization in acidic conditions (for instance poliphosphoric acid or acetic acid or mixtures of acetic and hydrochloric acids) applied to the hydrazone to form the ketocycloalkan[b]indole derivative as described for instance in Heterocycles 1986, 711 or Chem. Pharm. Bull. 1981, 699.

Route B outlines an alternative synthesis where, in step one, a classical Fischer indolization between a cycloalkanone and an aromatic hydrazine is performed under acidic conditions (for instance sulphuric acid in alcohol, a Lewis acid in tetrahydrofuran or neat trifluoroacetic anhydride) in order to achieve a cycloalkan[b]indole. In the subsequent step the cycloalkan[b]indole is oxidized to the corresponding ketocycloalkan[b]indole by means of a suitable oxidizing agent like, for instance, periodic acid or iodine pentoxide as described in J. Heterocyclic Chem. 2000, 37, 11 or Chem. Pharm. Bull. 1987, 35, 4700.

The synthetic pathways reported in the following schemes illustrate procedures that involve manipulation of functional groups on the ketocycloalkan[b]indole before pyrazole ring formation, i.e. the conversion of a compound of formula VI into a different compound of formula VI.

For example in Scheme VI, wherein R2, L, Ra and Rb are as described above, the preparation of derivatives of general formula (VI) containing acylamines as substituents is shown.

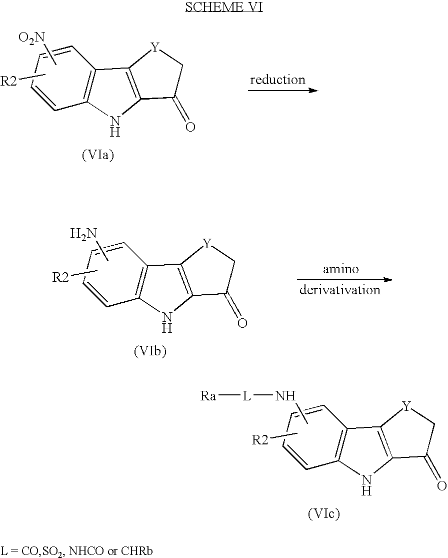

In step one the starting nitroketocycloalkan[b]indole, obtained as described above, after optional protection of the indole nitrogen with a suitable protecting group, is subdued to reduction of the nitro group, by means of well known methods, such as, for instance, chemical reduction with iron, zinc or tin(II) chloride treatment. The reaction may occur in a suitable solvent such as, for instance, N,N-dimethylformamide, 1,4-dioxane, ethanol/water, methanol/water, 1-methyl-2-pyrrolidinone or acetonitrile, at a temperature ranging from about −10° C. to reflux and for a suitable time, for instance from about 30 minutes to about 96 hours.

The said reduction may be also performed as a catalytic hydrogenation, according to conventional techniques, in the presence of a suitable catalyst such as, for instance, copper (II) acetate, palladium on charcoal or 4-dimethylaminopyridine.

In step two acylation of the amino group can occur reacting it with carboxylic acids or their derivatives, such as acyl chlorides and bromides, with sulphonic acid derivatives, namely sulphonyl chlorides and bromides, or with isocyanates to yield respectively carboxamido derivatives, sulphonamido derivatives or ureido derivatives.

The reaction between the aminoketocycloalkan[b]indole and a carboxylic acid can be carried out in the presence of a coupling agent such as, for instance, benzotriazol-1-yloxytris(pyrrolidino)phosphonium hexafluorophosphate, 1,3-dicyclohexylcarbodiimide, 1,3-diisopropylcarbodiimide, o-benzotriazol-1-yl-n,n,n′,n′-tetramethyluronium tetrafluoroborate, 1-(3-dimethylaminopropyl)-3-ethylcarbodimide, N-cyclohexylcarbodiimide-N′-propyloxymethyl polystyrene or N-cyclohexylcarbodiimide-N′-methyl polystyrene, in a suitable solvent such as, for instance, dichloromethane, chloroform, tetrahydrofuran, diethyl ether, 1,4-dioxane, acetonitrile, toluene or N,N-dimethylformamide, at a temperature ranging from about −10° C. to reflux and for a suitable time ranging from about 30 minutes to about 96 hours.

The said reaction is optionally carried out in the presence of a suitable catalyst, for instance 4-dimethylaminopyridine, or in the presence of a further coupling agent such as N-hydroxybenzotriazole. The reaction can also be carried out through a mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, acetonitrile, diethyl ether, 1,4-dioxane or N,N-dimethylformamide, and at a temperature ranging from about −30° C. to room temperature.

The reaction between the aminoketocycloalkan[b]indole and an acyl chloride or bromide can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, and at a temperature ranging from about −10° C. to reflux.

The reaction between the aminoketocycloalkan[b]indole and a sulphonyl derivative, such as the chloride or the bromide, can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, at a temperature ranging from about −10° C. to reflux.

Finally, the reaction between the aminoketocycloalkan[b]indole and an isocyanate can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile, or N,N-dimethylformamide, and at a temperature ranging from about −10° C. to reflux.

Alternatively the aminoketocycloalkan[b]indole is reacted under reductive conditions with a aldehyde or ketone derivative of formula RaRbCO so as to obtain the corresponding amine wherein Ra and Rb are as above defined. From the above, it is clear to the skilled man that by reacting an aldehyde derivative of RaCORb, for instance wherein Rb is a hydrogen atom, the corresponding derivative wherein L is a —CH₂— group will be obtained; likewise, by reacting a ketone derivative of formula RaCORb, a —CHRb— group will correspond to L.

This reaction, widely known as reductive alkylation of amines, occurs in the presence of a reducing agent such as, for instance, sodium borohydride, sodium cyanoborohydride or sodium triacetoxyborohydride, in a suitable solvent such as N,N-dimethylformamide, N,N-dimethylacetamide, chloroform, dichloromethane, tetrahydrofuran or acetonitrile, optionally in the presence of acetic acid, methanol or ethanol as co-solvents, at a temperature ranging from about −10° C. to the reflux temperature of the solvent and for a time varying from about 30 minutes to about 96 hours.

Finally, the ureido derivatives wherein Ra is hydrogen and L is (—NHCO—) may be prepared by reacting the aminoketocycloalkan[b]indole s with a suitable acylating agent, for instance triphosgene or trichloromethyl chloroformate, in the presence of aqueous or gaseous ammonia, according to conventional techniques.

The said reaction is carried out in a suitable solvent such as, for instance, dichloromethane, chloroform, toluene, tetrahydrofuran or dioxane, optionally in the presence of a tertiary base, for instance triethylamine, and of a catalyst such as 4-dimethylaminopyridine, at a temperature ranging from about −10° C. to room temperature and for a time varying from about 30 minutes to about 96 hours.

In Scheme VII, wherein Y is as described above and R4 is an optionally substituted phenyl group, like, for instance, 4-fluorophenyl, 4-methylphenyl and the like, the preparation of derivatives of general formula (VI) containing at one time the hydroxy and carboxy groups or the carboxy group at either the position 4 or 6 of the indole ring in the cycloalkan[b]indole as substituents is shown.

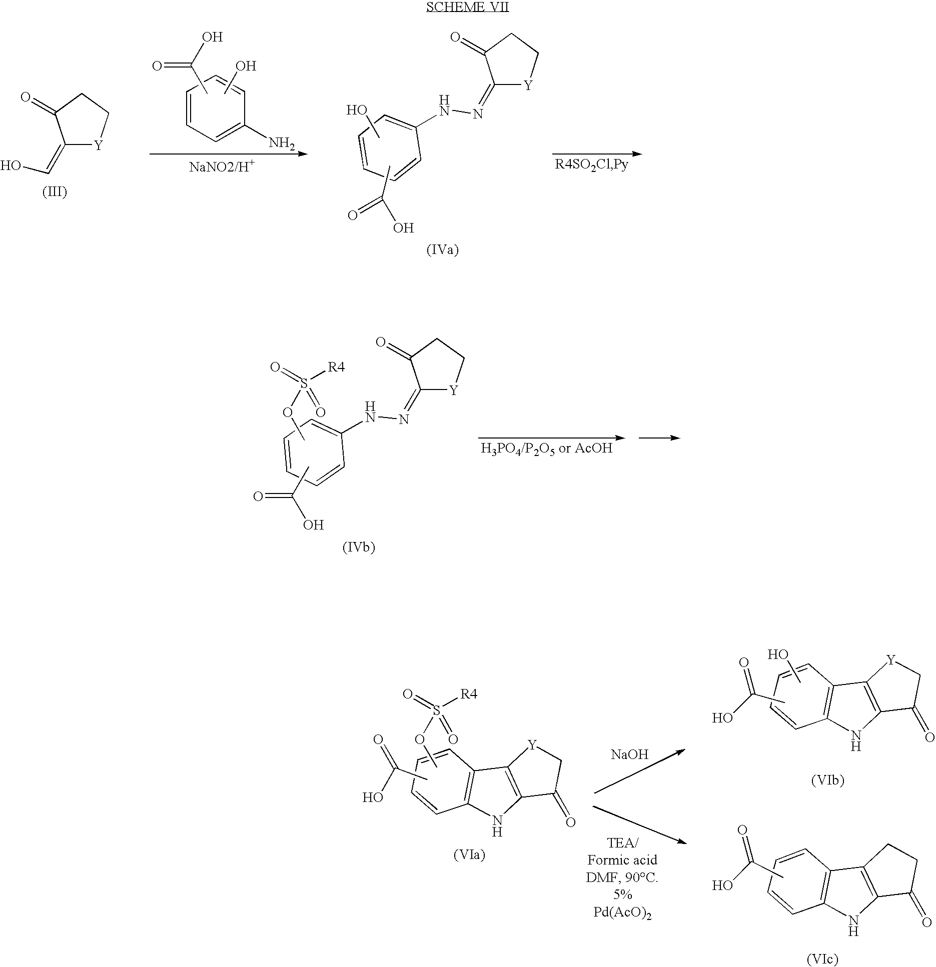

Here in stop one the aryldiazonium salt, prepared from a hydroxy and carboxy substituted aniline and sodium nitrite in acids, is added to a basic hydroalcoholic solution of the cycloalkanone derivative to yield the corresponding hydrazone, as described for instance in Chem. Pharm. Bull. 1981, 699. In step two the hydrazone derivative is reacted with an optionally substituted phenyl sulfonyl chloride in the presence of a suitable base, as described, for instance, in Tetrahedron, 1998, 54, 45, and the resulting sulfonate (IVb) is then subdued to Fischer indole cyclization as above. The ketocycloalkan[b]indole of general formula (VIa″), can be subdued to reduction, using triethylamine/Formic acid in the presence of Palladium acetate, as described, for instance in J. Org. Chem., 1990, 55, 350 to yield the ketocycloalkan[b]indole of general formula (VIc′) where the carboxy group is brought at either the positions 4 or 6 of the indole ring. Alternatively the ketocycloalkan[b]indole of general formula (VIa″), can be subdued to hydrolysis under basic conditions, using, for instance, sodium hydroxide in hydroalcoholic solutions, to furnish the ketocycloalkan[b]indole of general formula (VIb′).

The synthetic pathway reported in Scheme VIII illustrates a procedure for the preparation of derivatives of general formula (VI) containing carboxamides as substituents, wherein Rc, Rd, Y and R2 are as defined above.

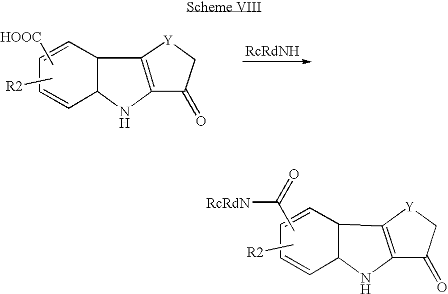

In scheme VIII the starting carboxyketocycloalkan[b]indole, obtained as above described, after optional protection of the indole nitrogen with a suitable protecting group, is reacted with an amine of formula RcRdNH, wherein Rc and Rd are as defined above, by means of well known methods, to give a compound of formula VI wherein R1 represents an optionally substituted group selected from C₁-C₆ alkylaminocarbonyl, C₁-C₆ dialkylaminocarbonyl, arylaminocarbonyl,hydroxyaminocarbonyl, C₁-C₆ alkyloxyaminocarbonyl, aryloxyaminocarbonyl, aminocarbonyl, and the like. For instance, the reaction can be carried out in the presence of a coupling agent such as, for instance, benzotriazol-1-yloxytris(pyrrolidino)phosphonium hexafluorophosphate, 1,3-dicyclohexylcarbodiimide, 1,3-diisopropylcarbodiimide, o-benzotriazol-1-yl-n,n,n′,n′-tetramethyluronium tetrafluoroborate, 1-(3-dimethylaminopropyl)-3-ethylcarbodiimide, N-cyclohexylcarbodiimide-N′-propyloxymethyl polystyrene or N-cyclohexylcarbodiimide-N′-methyl polystyrene, in a suitable solvent such as, for instance, dichloromethane, chloroform, tetrahydrofuran, diethyl ether, 1,4-dioxane, acetonitrile, toluene or N,N-dimethylformamide, at a temperature ranging from about −10° C. to the reflux temperature of the solvent and for a suitable time ranging from about 30 minutes to about 96 hours.

The said reaction is optionally carried out in the presence of a suitable catalyst, for instance 4-dimethylaminopyridine, or in the presence of a further coupling agent such as N-hydroxybenzotriazole. The reaction can also be carried out through a mixed anhydride method, that is by using an alkyl chloroformate such as ethyl, isobutyl, or isopropyl chloroformate, in the presence of a tertiary base such as triethylamine, N,N-diisopropylethylamine or pyridine, in a suitable solvent such as toluene, dichloromethane, chloroform, tetrahydrofuran, acetonitrile, diethyl ether, 1,4-dioxane or N,N-dimethylformamide, and at a temperature ranging from about −30° C. to room temperature.

Alternatively the carboxy group can be activated by transforming it, for example, in an acyl chloride by means of thionyl chloride or oxalyl chloride in a suitable solvent such as tetrahydrofuran, N,N-dimethylformamide at a temperature ranging from about −10° C. to the reflux temperature of the solvent and for a suitable time ranging from about 30 minutes to about 96 hours.

The reaction between the ketocycloalkan[b]indole carbonyl chloride or bromide and a primary (Rc=H) or secondary amine can be carried out in the presence of a tertiary base such as triethylamine, N,N-diisopropylethylamine or pyridine, in a suitable solvent such as toluene, dichloromethane, chloroform, diethyl ether, tetrahydrofuran, acetonitrile or N,N-dimethylformamide, and at a temperature ranging from about −10° C. to the reflux temperature of the solvent. The starting compounds of the formula (II), (III), (X), (XI), (XII), (XIII) and (XIV) are known or can be prepared starting from known compounds using known methods of preparation.

Pharmacology

The compounds of formula (I) are active as protein kinase inhibitors and may be therefore useful, for instance, to restrict the unregulated proliferation of tumour cells. In therapy, they may be used in the treatment of various tumours, such as those formerly reported, as well as in the treatment of other cell proliferative disorders such as psoriasis, vascular smooth cell proliferation associated with atherosclerosis and post-surgical stenosis and restenosis and in the treatment of Alzheimer's disease.

The inhibiting activity of putative cdk/cyclin inhibitors and the potency of selected compounds is determined through a method of assay based on the use of the SPA technology (Amersham Pharmacia Biotech).

The assay consists of the transfer of radioactivity labelled phosphate moiety by the kinase to a biotinylated substrate. The resulting 33P-labelled biotinylated product is allowed to bind to streptavidin-coated SPA beads (biotin capacity 130 pmol/mg), and light emitted was measured in a scintillation counter.

Inhibition Assay of cdk2/Cyclin A Activity

Kinase reaction: 4 μM in house biotinylated histone H1 (Sigma # H-5505) substrate, 10 μM ATP (0.1 microCi P³³γ-ATP), 1.1 nM Cyclin A/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 60 min at room temperature, the reaction was stopped by addition of 100 μL PBS buffer containing 32 mM EDTA, 500 μM cold ATP, 0.1% Triton X100 and 10mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 determination: inhibitors were tested at different concentrations ranging from 0.0015 to 10 μM. Experimental data were analyzed by the computer program GraphPad Prizm using the four parameter logistic equation:
y=bottom+(top−bottom)/(1+10ˆ((log IC50−x)*slope))

where x is the logarithm of the inhibitor concentration, y is the response; y starts at bottom and goes to top with a sigmoid shape.

Ki Calculation:

Experimental method: Reaction was carried out in buffer (10 mM Tris, pH 7.5, 10 mM MgCl₂, 0.2 mg/ml BSA, 7.5 mM DTT) containing 3.7 nM enzyme, histone and ATP (constant ratio of cold/labeled ATP 1/3000). Reaction was stopped with EDTA and the substrate captured on phosphomembrane (Multiscreen 96 well plates from Millipore). After extensive washing, the multiscreen plates were read on a top counter. Control (time zero) for each ATP and histone concentrations was measured.

Experimental design: Reaction velocities are measured at four ATP, substrate (histone) and inhibitor concentrations. An 80-point concentration matrix was designed around the respective ATP and substrate Km values, and the inhibitor IC50 values (0.3, 1, 3, 9 fold the Km or IC50 values). A preliminary time course experiment in the absence of inhibitor and at the different ATP and substrate concentrations allows the selection of a single endpoint time (10 min) in the linear range of the reaction for the Ki determination experiment

Kinetic parameter estimates: Kinetic parameters were estimated by simultaneous nonlinear least-square regression using [Eq.1] (competitive inhibitor respect to ATP, random mechanism) using the complete data set (80 points):

$$v = \frac{Vm \cdot A \cdot B}{\begin{matrix}α \cdot Ka \cdot Kb + α \cdot Ka \cdot B + \\ a \cdot Kb \cdot A + A \cdot B + α \cdot \frac{Ka}{ Ki} \cdot I \cdot ( Kb + \frac{B}{ β} )\end{matrix}} \qquad [ Eq . 1 ]$$

where A=[ATP], B=[Substrate], I=[inhibitor], Vm=maximum velocity, Ka, Kb, Ki the dissociation constants of ATP, substrate and inhibitor respectively. α and β the cooperativity factor between substrate and ATP binding and substrate and inhibitor binding respectively.

In addition the selected compounds are characterized on a panel of ser/thre kinases strictly related to cell cycle (cdk2/cyclin E, cdk1/cyclin B1, cdk5/p25,cdk4/cyclin D1), and also for specificity on MAPK, PKA, EGFR, IGF1-R, Aurora-2 and Cdc 7.

Inhibition Assay of cdk2/Cyclin E Activity

Kinase reaction: 10 μM in house biotinylated histone H1 (Sigma # H-5505) substrate, 30 μM ATP (0.3 microCi P³³γ-ATP), 4 ng GST-Cyclin E/CDK2 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 60 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μL of 5M CsCl. After 4 hours, the plates were read for 2 min in a Packard TOP-Count radioactivity leader.

IC50 Determination: See Above

Inhibition Assay of cdk1/Cyclin B1 Activity

Kinase reaction: 4 μM in house biotinylated histone H1 (Sigma # H-5505) substrate, 20 μM ATP (0.2 microCi P³³γ-ATP), 3 ng Cyclin B/CDK1 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate. After 20 min. incubation for substrate capture, 100 μl 5M CsCl were added to allow statification of beads to the top of the Optiplate and let stand 4 hours before radioactivity counting in the Top-Count instrument

IC50 Determination: See Above

Inhibition Assay of cdk5/p25 Activity

The inhibition assay of cdk5/p25 activity is performed according to the following protocol.

Kinase reaction: 10 μM biotinylated histone H1 (Sigma # H-5505) substrate, 30 μM ATP (0.3 microCi P³³γ-ATP), 15 ng CDK5/p25 complex, inhibitor in a final volume of 30 μl buffer (TRIS HCl 10 mM pH 7.5, MgCl₂ 10 mM, DTT 7.5 DIM+0.2 mg/ml BSA) were added to each well of a 96 U bottom. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 Determination: See Above

Inhibition Assay of cdk4/Cyclin D1 Activity

Kinase reaction: 0.4 μM mouse GST-Rb (769-921) (# sc-4112 from Santa Cruz) substrate, 10 μM ATP (0.5 μCi P³³γ-ATP), 100 ng of baculovirus expressed GST-cdk4/GST-Cyclin D1, suitable concentrations of inhibitor in a final volume of 50 μl buffer (TRIS HCl 10 mM pH 7.5, MgCl₂ 10 mM, 7.5 mM DTT+0.2 mg/ml BSA) were added to each well of a 96 U bottom well plate. After 40 min at 37° C. incubation, reaction was stopped by 20 μl EDTA 120 mM.

Capture: 60 μl were transferred-from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled Rb fragment was detected by radioactivity counting in the Top-Count instrument.

IC50 Determination: See Above

Inhibition Assay of MAPK Activity

Kinase reaction: 10 μM in house biotinylated MBP (Sigma # M-1891) substrate, 15 μM ATP (0.15 microCi P³³γ-ATP), 30 ng GST-MAPK (Upstate Biothecnology # 14-173), inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After incubation for 35 min at room temperature, the reaction was stopped by addition of 100 μl PBS buffer containing 32 mM EDTA, 500 μM cold ATP, 0.1% Triton X100 and 10mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 Determination: See Above

Inhibition Assay of PKA Activity

Kinase reaction: 10 μM in house biotinylated histone H1 (Sigma # H-5505) substrate, 10 μM ATP (0.2 microM P³³γ-ATP), 0.45 U PKA (Sigma # 2645), inhibitor in a final volume of 30 μl buffer (TRIS HCl 10 mM pH 7.5, MgCl₂ 10 mM DTT 7.5 mM+0.2 mg/ml BSA) were added to each well of a 96 U bottom. After incubation for 90 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 Determination: See Above

Inhibition Assay of EGFR Activity

Kinase reaction: 10 μM in house biotinylated MBP (Sigma # M-1891) substrate, 2 μM ATP (0.04 microCi P³³γ-ATP), 36 ng insect cell expressed GST-EGFR, inhibitor in a final volume of 30 μl buffer (Hepes 50 mM pH 7.5, MgCl₂ 3 mM, MnCl₂ 3 mM, DTT 1 mM, NaVO₃ 3 μM,+0.2 mg/ml BSA) were added to each well of a 96 U bottom. After incubation for 20 min at room temperature, the reaction was stopped by addition of 100 μl PBS buffer containing 32 mM EDTA, 500 μM cold ATP, 0.1% Triton X100 and 10mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 Determination: See Above

Inhibition Assay of IGF1-R Activity

The inhibition assay of IGF1-R activity is performed according to the following protocol.

Enzyme activation: IGF1-R must be activated by auto-phosphorylation before starting the experiment. Just prior to the assay, a concentrated enzyme solution (694 nM) is incubated for half a hour at 28° C. in the presence of 100 μM ATP and then brought to the working dilution in the indicated buffer.

Kinase reaction: 10 μM biotinylated IRS1 peptide (PRIMM) substrate, 0-20 μM inhibitor, 6 μM ATP, 1 microCi ³³P-ATP, and 6 nM GST-IGF1-R (pre-incubated for 30 min at room temperature with cold 60 μM cold ATP) in a final volume of 30 μl buffer (50 mM HEPES pH 7.9, 3 mM MnCl₂, 1 mM DTT, 3 μM NaVO₃) were added to each well of a 96 U bottom well plate. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

Inhibition Assay of Aurora-2 Activity

Kinase reaction: 8 μM biotinylated peptide (4 repeats of LRRWSLG), 10 μM ATP (0.5 uCi P³³γ-ATP), 7.5 ng Aurora 2, inhibitor in a final volume of 30 μl buffer (HEPES 50 mM pH 7.0, MgCl₂ 10 mM, 1 mM DTT, 0.2 mg/ml BSA, 3 μM orthovanadate) were added to each well of a 96 U bottom well plate. After 60 minutes at room temperature incubation, reaction was stopped and biotinylated peptide captured by adding 100 μl of bead suspension.

Stratification: 100 μl of CsCl 2 5 M were added to each well and let stand 4 hour before radioactivity was counted in the Top-Count instrument.

IC50 Determination: See Above

Inhibition Assay of Cdc7/dbf4 Activity

The inhibition assay of Cdc7/dbf4 activity is performed according to the following protocol.

The Biotin-MCM2 substrate is trans-phosphorylated by the Cdc7/Dbf4 complex in the presence of ATP traced with γ³³-ATP. The phosphorylated Biotin-MCM2 substrate is then captured by Streptavidin-coated SPA beads and the extent of phosphorylation evaluated by β counting.

The inhibition assay of Cdc7/dbf4 activity was performed in 96 wells plate according to the following protocol.

To each well of the plate were added:
10 μl substrate (biotinylated MCM2, 6 μM final concentration)
10 μl enzyme (Cdc7/Dbf4, 17.9 nM final concentration)
10 μl test compound (12 increasing concentrations in the nM to μM range to generate a dose-response curve)
10 μl of a mix of cold ATP (2 μM final concentration) and radioactive ATP (1/5000 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 μM NaVO₃, 2mM glycerophosphate and 0.2 mg/ml BSA. The solvent for test compounds also contained 10% DMSO.

After incubation for 60 minutes, the reaction was stopped by adding to each well 100 μl of PBS pH 7.4 containing 50 mM EDTA, 1 mM cold ATP, 0.1% Triton X100 and 10 mg/ml streptavidin coated SPA beads.

After 20 min incubation, 110 μL of suspension were withdrawn and transferred into 96-well OPTIPLATEs containing 100 μl of 5M CsCl. After 4 hours, the plates were read for 2 min in a Packard TOP-Count radioactivity reader.

IC50 Determination: See Above.

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from about 10 to about 500 mg pro dose, from 1 to 5 times daily. The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase II inhibitors and the like, optionally within liposomal formulations thereof. If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

The present invention also includes pharmaceutical compositions comprising a compound of formula (I) or a pharmaceutically acceptable salt thereof in association with a pharmaceutically acceptable excipient (which can be a carrier or a diluent).

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatin, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulfates; and, in general, non-toxic and pharmacologically inactive substances used in pharmaceutical formulations. Said pharmaceutical preparations may be manufactured in known manner, for example, by means of mixing, granulating, tabletting, sugar-coating, or film-coating processes.

The liquid dispersions for oral administration may be e.g. syrups, emulsions and suspensions.

The syrups may contain as carrier, for example, saccharose or saccharose with glycerin and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

### Experimental Part

General Methods

Flash chromatografy was performed on silica gel (Merck grade 9385, 60Å). HPLC/MS was performed on a Waters X Terra RP 18 (4.6×50 mm, 3.5 μm) column using a Waters 2790 HPLC system equipped with a 996 Waters PDA detector and a Micromass mod. ZQ single quadrupole mass spectrometer, equipped with an electrospray (ESI) ion source. Mobile phase A was ammonium acetate 5 mM buffer (pH 5.5 with acetic acid/acetonitrile 95:5), and Mobile phase B was H₂O/acetonitrile (5:95). Gradient from 10 to 90% B in 8 minutes, hold 90% B 2 min. UV detection at 220 nm and 254 nm. Flow rate 1 ml/min. Injection volume 10 μl. Full scan, mass range from 100 to 800 amu. Capillary voltage was 2.5 KV; Source temp. was 120° C.; Cone was 10 V. Retention Times (HPLC Rt) are given in minutes at 220 nm or 254 nm. Mass are given as m/z ratio.

When necessary compounds have been purified by Preparative HPLC on a Waters Symmetry C18 (19×50 mm, 5 um) column using a Waters preparative HPLC 600 equipped with a 996 Waters PDA detector and a Micromass mod. ZMD single quadrupole mass spectrometer, electrospray ionisation, positive mode. Mobile phase A was water 0.01% TFA, and Mobile phase B was acetonitrile. Gradient from 10 to 90% B in 8 min, hold 90% B 2 min. Flow rate 20 ml/m.

¹H-NMR spectroscopy was performed on a Mercury VX 400 operating at 400.45 MHz equipped with a 5 mm double resonance probe (1H (15N-31P) ID_PFG Varian).

### EXAMPLES

The following examples are herewith intended to better illustrate the present invention without posing any limitation to it.

Preparation of Compounds of General Formula IV

### Preparation a

### Cyclohexane-1,2-dione (4-nitrophenyl)hydrazone (IVa)

To a stirred solution of 4-nitroaniline (4 g, 29 mmol) in 37% aqueous HCl (9 mL), cooled between −5 and 0° C., a cooled solution of sodium nitrite (4.4 g, 63.8 mmol) in 18 mL of water was slowly dropped, maintaining the temperature between −5 and 0° C. After addition the cooled solution was stirred for 30′ and slowly added to a cold (−5° C.) solution of 2-hydroxymethylenecyclohexanone (4 g, 31.7 mmol) and sodium acetate (6.4 g), in a mixture of methanol (35 mL) and water (160 mL).

During addition a bright yellow precipitate formed. After stirring 30′ at 0° C. the precipitate was filtered and washed thoroughly with water. After drying, the desired hydrazone was obtained as a yellow solid (6.8 g, 27 mml, 92% yield). ¹H-NMR (DMSOd₆), diagnostic signals (ppm): 10.4 (a, 1H), 8.2 (d, 2H), 7.4 (d, 2H), 2.7 (m, 2H), 2.5 (m, 2H), 1.8 (m, 4H).

[M+H]⁺=248

### Preparation b

### 4-[2-(2-Oxocyclohexylidene)hydrazino]benzoic acid (IVb)

The compound was prepared as described above for cyclohexane-1,2-dione (4-nitrophenyl)hydrazone, by using the appropriate aniline derivative. Yellow solid in 70% yield.

¹H-NMR (DMSOd₆), diagnostic signals (ppm): 10.1 (s, 1H), 7.9 (d, 2H), 7.3 (d, 2H), 2.6 (m, 2H), 2.4 (m, 2H), 1.8 (m, 4H).

[M+H]⁺=247

### Preparation c

### 3-[2-(2-Oxocyclohexylidene)hydrazino]benzoic acid (IVc)

The compound was prepared as described above for cyclohexane-1,2-dione (4-nitrophenyl)hydrazone, by using the appropriate aniline derivative. Yellow solid (88% yield).

¹H-NMR (DMSOd₆), diagnostic signals (ppm): 10.0 (s, 1H), 7.9 (s, 1H), 7.5-7.35 (m, 3H), 2.6 (t, 2H), 2.4 (t, 2H), 1.8 (m, 4H).

[M+H]⁺=247

### Preparation d

### 4-hydroxy-3-[2-(2-oxocyclohexylidene)hydrazino]benzoic acid (IVd)

A solution of diazonium salt was prepared from 1 gram (6.5 mmoles) of 3-amino-4-hydroxybenzoic acid and 0.46 grams (6.6 mmoles) of sodium nitrite in 5 mL of water. With constant stirring at room temperature, 2.1 grams of conc. HCl was added to the above. This diazonium salt solution was added dropwise to a solution of 0.8 grams (6.6 mmoles) of 2-hydroxymethylenecyclohexanone and 0.4 grams (6.8 mmoles) of 50% KOH aq. (KOH: H2O=0.4 grams: 0.4 mL) in 10 mL of EtOH stirred at 0° C.

Upon complete diazonium salt addition, the system was stirred an additional 30 minutes at room temperature at which time the thick, red-colored product was poured into 30 mL of water. The dark red precipitates were collected by filtration, washed once with 10 mL of water and dried under vacuum. 1.53 grams of product was so isolated (89%). [M+H]+=263

### Preparation e

### 4-toluenesulfonyloxy-3-[2-(2-oxocyclohexylidene)hydrazino]benzoic acid (IVe)

0.10 grams (0.38 mmoles) of [9-hydroxy-3,4,-dihydrocarbazole-1(2H)-one-5-carboxy late] was dissolved in 2 mL of pyridine to which 1.3 equivalents, (0.09 grams, 0.49 mmoles) of p-toluenesulfonyl chloride was added. The mixture was stirred at room temperature for 16 hours after which time the solvent volume was reduced under vacuum. The crude, red oil was re-dissolved in 10 mL of DCM and washed twice with 10 mL of saturated brine solution. Upon evaporation of the organic solvent, 0.11 grams (76%) of red, crystalline crude product was obtained.

[M+H]⁺=417

### Preparation f

### 4-fluorobenzenesulfonyloxy-3-[(2Z)-2-(2-oxocyclohexylidene)hydrazino]benzoic acid (IVf)

Prepared using the protocol described for 4-toluenesulfonyloxy-3-[2-(2-oxocyclohexylidene)hydrazino]benzoic acid. Here, 0.20 grams (0.76 mmoles) of [9-hydroxy-3,4,-dihydrocarbazole-1(2H)-one-5-carboxy late] and 1.3 equivalents, (0.19 grams, 1.0 mmoles) of 4-fluorobenzenesulfonyl chloride in 4 mL of pyridine were used.

After extraction and solvent evaporation, 0.25 grams (74%) of red, crystalline crude product was obtained.

[M+H]⁺=421

Compounds of General Formula VI

### Preparation g

### 6-Nitro-2,3,4,9-tetrahydro-1H-carbazol-1-one (VIa)

Cyclohexane-1,2-dione (4-nitrophenyl)hydrazone (Iva) (5 g, 20.4 mmol) was added to freshly prepared polyphosphoric acid, obtained mixing under vigorous magnetic stirring P₂O₅ (10 g) and H₃PO₄ (40 mL), and the thick mixture was stirred at 120-125° C. for 45′. Heating was removed and the mixture was let to cool to room temperature before pouring it into 200 mL of stirred water. After 30′ stirring the brown precipitate was filtered. The filtrate was extracted (×4) with ethyl acetate, the organic phase dried over sodium sulphate and concentrated under reduced pressure. The combined raw solids (3.5 g) were purified by silica gel chromatography, eluting with dichloromethane/methanol 25:1. Obtained an orange solid (2.2 g, 9.6 mmol, 47% yield). ¹H-NMR (DMSOd₆), diagnostic signals (ppm): 12.3 (s,m 1H), 8.7 (s, 1H), 8.1 (d, 1H), 7.5 (d, 1H), 3.0 (t, 2H), 2.6 (t, 2H), 2.15 (m, 2H).

M+H]⁺=231

### Preparation h

### 1-Oxo-2,3,4,9-tetrahydro-1H-carbazole-6-carboxylic acid (VIc)

The compound was prepared as described above for 6-nitro-2,3,4,9-tetrahydro-1H-carbazol-1-one, without need for silica gel chromatography. Obtained a brownish solid (94% yield).

¹H-NMR (DMSOd₆), diagnostic signals (ppm): 8.35 (s, 1H), 7.9 (dd, 2H), 7.4 (d, 1H), 3.0 (t, 2H), 2.6 (t, 2H), 2.2 (t, 2H).

[M+H]⁺=230

### Preparation i

### 1-Oxo-2,3,4,9-tetrahydro-1H-carbazole-5-carboxylic acid (VId)+1-oxo-2,3,4,9-tetrahydro-1H-carbazole-7-carboxylic acid (VIe)

The compound was prepared as described above for 6-nitro-2,3,4,9-tetrahydro-1H-carbazol-1-one, purifying the crude by silica gel chromatography, eluting with dichloromethane/methanol 15:1. Obtained a whitish solid, mixture of the two regioisomeric acids (34% yield).

¹H-NMR (DMSOd6), diagnostic signals (ppm): 11.9 (s, 2H), 8.05 (s, 2H), 7.75 (d, 2H), 7.7-7-3 (m, 4H), 7.5-7.35 (m, 3H), 3.15 (m, 2H), 2.95 (t, 2H), 2.5 (m, 4H), 2.1 (m, 4H).

[M+H]⁺=230

### Preparation j

### 6-Amino-2,3,4,9-tetrahydro-1H-carbazol-1-one (VIf)

A suspension of 6-nitro-2,3,4,9-tetrahydro-1H-carbazol-1-one (VIa, 0.12 g, 0.5 mmol) in methanol (3 mL), water (1 mL), powdered iron (0.08 g) and ammonium chloride (0.12 g) was refluxed for 2 hrs under vigorous stirring. The reaction mixture was cooled, filtered through dicalite, concentrated and dissolved in a mixture of dichloromethane/water. The organic phase was washed with water, dried over sodium sulphate and concentrated under reduced pressure to give a yellow solid (0.07 g, 0.35 mmol, 70% yield).

[M+H]⁺=201

### Preparation k

### N-(1-oxo-2,3,4,9-tetrahydro-1H-carbazol-6-yl)acetamide (VIg)

To a solution of 6-amino-2,3,4,9-tetrahydro-1H-carbazol-1-one (VIf, 0.1 g, 0.5 mmol) in dry tetrahydrofuran (2 mL) and DIPEA (0.2 mL) acetyl chloride (0.07 mL, 1 mmol) was added at room temperature under stirring. After 1 hr at room temperature the mixture was concentrated and dissolved in ethyl acetate. The organic phase was washed with water, dried over sodium sulphate and concentrated under reduced pressure to give, after precipitation in ether, a whitish solid (0.11 g, 0.45 mmol, 90% yield).

¹H-NMR (DMSOd₆), diagnostic signals (ppm): 11.4 (s, 1H), 9.8 (s, 1H), 7.95 (s, 1H), 7.3 (m, 2H), 2.8 (t, 2H), 2.5 (t, 2H), 2.15 (m, 2H), 2.0 (s, 3H).

[M+H]⁺=243

### Preparation 1

### 3-Methyl-N-(1-oxo-2,3,4,9-tetrahydro-1H-carbazol-6-yl)butanamide (VIh)

The compound was prepared as described for N-(1-oxo-2,3,4,9-tetrahydro-1H-carbazol-6-yl)acetamide as a brownish solid in 78% yield after precipitation from ether.

¹H-NMR (DMSOd₆), diagnostic signals (ppm): 11.45 (s, 1H), 9.7 (s, 1H), 8.0 (s, 1H), 7.3 (dd, 2H), 2.9 (t, 2H), 2.55 (t, 2H), 2.2-2.0 (m, 5H), 0.9 (d, 6H).

[M+H]⁺=285

### Preparation m

### N-isobutyl-1-oxo-2,3,4,9-tetrahydro-1H-carbazole-6-carboxamide (VIi)

To a solution of 1-oxo-2,3,4,9-tetrahydro-1H-carbazole-6-carboxylic acid (see example 2, 0.46 g, 2 mmol) in dry tetrahydrofuran (20 mL) and dimethylformamide (2 drops), cooled at 0° C. under argon atmosphere, oxalyl chloride (1 mL, 11.8 mmol) was added dropwise. After addition the reaction mixture was let to warm to room temperature and stirred for 1 hr. The reaction mixture was concentrated to dryness under reduced pressure and dissolved in dichloromethane (20 mL) and DMAP (0.2 mL, 1.2 mmol). To this solution at room temperature isobutylamine (0.5 mL, 5 mmol) was slowly added. The reaction mixture was stirred at room temperature for 2 hr, and then it was concentrated. The crude material was purified by flash chromatography (eluant: CH₂Cl₂/MeOH 30:1) to give a yellow solid (0.14 g, 0,46 mmol, 23% two step yield).

¹H-NMR (DMSOd₆), diagnostic signals (ppm): 11.8 (s, 1H), 8.35 (t, 1H), 8.24 (s, 1H), 7.8 (d, 1H), 7.4 (d, 1H), 3.1 (t, 2H), 3.0 (t, 2H), 2.6 (t, 2H), 2.15 (m, 2H), 1.85 (m, 1H), 0.9 (d, 6H).

M+H]⁺=285

### Preparation n

### N-(1-oxo-2,3,4,9-tetrahydro-1H-carbazol-7-yl)thiophene-2-carboxamide (VIj)

The compound was prepared as described for N-(1-oxo-2,3,4,9-tetrahydro-1H-carbazol-6-yl)acetamide, as a brownish solid in 74% yield after precipitation from ether. ¹H-NMR (DMSOd₆), diagnostic signals (ppm): 11.5 (s, 1H), 10.2 (s, 1H), 8.0 (m, 2H), 7.8 (d, 1H), 7.45 (dd, 1H), 7.35 (d, 1H), 7.2 (m, 1H), 2.9(t, 2H), 2.55 (t, 2H), 2.15 (m, 2H). [M+H]⁺=311

### Preparation o

### 6-[(4-Methylpiperazin-1-yl)carbonyl]-2,3,4,9-tetrahydro-1H-carbazol-1-one (VIl)

The product was obtained as described for N-isobutyl-1-oxo-2,3,4,9-tetrahydro-1H-carbazole-6-carboxamide, after flash chromatography (eluant: CH₂Cl₂/MeOH 20:1), as a yellowish solid in 45% yield.

¹H-NMR (DMSOd₆), diagnostic signals (ppm): 11.75 (s, 1H), 7.7 (s, 1H), 7.4 (d, 1H), 7.3 (d, 1H), 3.5 (bs, 4H), 2.95 (t, 2H), 2.55 (t, 2H), 2.3 (bs, 4H), 2.2 (s, 3H), 2.15 (m, 2H). [M+H]+=312

### Preparation p

### 8-Oxo-1-(toluene-4-sulfinyloxy)-6,7,8,9-tetrahydro-5H-carbazole-4-carboxylic acid (VIm)

4-toluenesulfonyloxy-3-[2-(2-oxocyclohexylidene)hydrazino]benzoic acid, (0.10 grams, 0.24 mmoles) was mixed with 1 mL of polyphosphoric acid (PPA). The mixture was heated to 80° C. with stirring for 1 hour. The black colored reaction mixture was diluted with water to 2 mL and stirred at 80° C. for an additional 30 minutes. The mixture was allowed to cool to room temperature and diluted again with 2 mL of water. Following extraction three times, each with 3 mL of ethyl acetate, the combined organic fractions were dried over sodium sulfate and the solvent removed under vacuum. After extraction and solvent evaporation, 0.073 grams (76%) of dark brown oil was obtained.

[M+H]⁺=400

### Preparation q

### 8-Oxo-1-(fluorobenzene-4-sulfinyloxy)-6,7,8,9-tetrahydro-5H-carbazole-4-carboxylic acid (VIn)

Prepared using the protocol indicated for 8-oxo-1-(toluene-4-sulfinyloxy)-6,7,8,9-tetrahydro-5H-carbazole-4-carboxylic acid.

After extraction and solvent evaporation, 0.18 grams (89%) of dark brown to black oil was obtained.

This product was purified by column chromatography over 5 grams of silica gel with hexane-ethyl acetate. A single fraction, containing predominantly desired product was isolated. Here, 0.062 grams (30%) of yellow to brown crystalline product was isolated with the correct structural identity.

[M+H]⁺=404

### Preparation r

### 9-(Tetrahydro-pyran-2-yl)-2,3,4,9-tetrahydro-carbazol-1-one (VIo)

To 15 ml of a solution of 2,3,4,9-tetrahydro-carbazol-1-one (1 g, 5.4 mmol) in dichloromethane 3,4-dihydro-2H-pyran (0.681 g, 8.1 mmol) was added. After the addition of 50 mg (0.25 mmol) of p-toluenesulphonic acid the solution was stirred at room temperature for six hours. After evaporation of the solvent the crude was purified by silica gel chromatography eluting with hexane-ethyl acetate 8-2, yielding 645 mg (50% yield) of product as a colourless solid.

[M+H]⁺=270

### Preparation s

### Methyl 1-oxo-2,3,4,9-tetrahydro-1H-carbazole-6-carboxylate (VIP)

A suspension of methyl 1-oxo-2,3,4,9-tetrahydro-1H-carbazole-6-carboxylic acid (8 g, 34.9 mmol) in methanol (800 mL) and sulforic acid (2 ml) was refluxed for 16 hours under vigorous stirring. The reaction mixture was cooled, and a solution of NaHCO₃ 10% in water (100 ml) was added, methanol was removed under reduced pressure and the aqueous phase was extracted with ethyl acetate (3×100 ml). The organic phase was dried over sodium sulphate and concentrated under reduced pressure to give a brown solid (7.7 g, 31 mmol, 90% yield).

[M+H]+=244

¹H-NMR (DMSOd6), diagnostic signals (ppm): 2.00 (s, 3H), 2.15 (m, 2H), 2.50 (t, 2H), 2.80 (t, 2H), 7.3 (m, 2H), 7.95 (a, 1H), 9.80 (s, 1H), 11.40 (s, 1H).

### Preparation t

### 1-Oxo-1,2,3,4-tetrahydro-9H-carbazole-5-carboxylic acid (VId)

0.060 grams (0.15 mmoles) of (8-fluorobenzenesulfonyloxy-1,2,3,4,-tetrahydro-9H-carbazole-5-carboxylic acid] were dissolved in 1 mL of degassed dimethylformamide. Under an argon atmosphere at room temperature were sequentially added Et3N (0.075 g, 0.75 mmoles), formic acid (0.034 g, 0.075 mmoles), 1,3-bis(diphenylphosphino)propane (DPPP) (018 g, 0.0075 mmoles) and Pd(AcO)₂ (0.0017 g, 0.0075 mmoles). The reaction temperature was raised to 90oC for 1 hour. After 1 hour, the reaction mixture was cooled to room temperature where 5 mL of DCM was added. The system was washed two times with 10 mL of 5% aqueous hydrochloric acid followed by one wash with water. The organic layer was passed through a 2 gram silica plug and washed with and additional 5 mL portion of DCM. Elution of the desired product from the column was accomplished with 20 mL of ethyl acetate. Upon solvent removal, 0.043g of a light brown solid was isolated.

[M+H]+=230

Compounds of General Formula VII

### Preparation u

### 2-[(Dimethylamino)methylene]-6-nitro-2,3,4,9-tetrahydro-1H-carbazol-1-one (VIIa)

6-Nitro-2,3,4,9-tetrahydro-1H-carbazol-1-one (VIa, 1 g, 4.34 mmol) and N,N-dimethylformamide diethylacetal (30 mL, 180 mmol) were stirred at 110-115° C., distilling off the ethanol formed. After 90′ the temperature was raised to reflux for 1 hr. After cooling the precipitate was filtered and washed with diethyl ether. Obtained an orange solid (0.9 g, 3.15 mmol, 72% yield).

¹H-NMR (DMSOd₆), diagnostic signals (ppm): 12.1 (a, 1H), 8.6 (s, 1H), 8.05 (d, 1H), 7.5 (d, 2H), 3.12 (s, 6H), 3.05 (t, 2H), 2.85 (t, 2H).

[M+H]⁺=286

### Preparation v

### 2-(Hydroxymethylene)-1-oxo-2,3,4,9-tetrahydro-1H-carbazole-6-carboxylic acid (VIIb)

To 1-oxo-2,3,4,9-tetrahydro-1H-carbazole-6-carboxylic acid (VIc, 0.23 g, 1 mmol), dissolved in anhydrous dimethylformamide (5 mL), 95% NaE (0.24 g, 10 mmol) was added and the reaction mixture stirred at room temperature for 15′. Ethyl formate (1 mL, 12.4 mmol) was added dropwise and slowly and the reaction mixture stirred at room temperature for 1 hr. The reaction mixture was poured onto ice-cooled 2N hydrochloric acid (7 mL) under stirring. The dark precipitate was filtered, washed with water, dried and used in the subsequent step.

[M+H]⁺=258

### Preparation w

### 2-(Hydroxymethylene)-1-oxo-2,3,4,9-tetrahydro-1H-carbazole-5-carboxylic acid (VIIc) and

### 2-(hydroxmethylene)-1-oxo-2,3,4,9-tetrahydro-1H-carbazole-7-carboxylic acid) (VIId)

The compounds were prepared as described above for 2-(hydroxymethylene)-1-oxo-2,3,4,9-tetrahydro-1H-carbazole-6-carboxylic acid. The precipitate was extracted (three times) with ethyl acetate, washed with water, dried over sodium sulphate and concentrated to yield a yellow solid, mixture of the two regioisomeric acids, that was used in the subsequent step without further purification.

[M+H]⁺=258

### Preparation y

### Ethyl 2-hydroxy(1-oxo-1,3,4,9-tetrahydro-2H-carbazol-2-ylidene)ethanoate (VIIe)

The compound was prepared as described above for 2-(hydroxymethylene)-1-oxo-2,3,4,9-tetrahydro-1H-carbazole-6-carboxylic acid, reacting diethyloxalate in place of ethylformate with commercially available 2,3,4,9-tetrahydro-1H-carbazol-1-one. Obtained a yellow solid in 77% yield.

¹H-NMR (DMSOd₆), diagnostic signals (ppm): 7.7 (d, 1H), 7.4 (m, 2H), 7.2 (m, 1H), 4.4 (q, 2H), 3.25 (t, 2H), 3.05 (t, 2H), 1.4 (t, 3H).

[M+H]⁺=286

### Preparation x

### N-{2-[(dimethylamino)methylene]-1-oxo-2,3,4,9-tetrahydro-1H-carbazol-6-yl}acetamide (VIIf)

The compound was prepared as described for 2-[(dimethylamino)methylene]-6-nitro-2,3,4,9-tetrahydro-1H-carbazol-1-one, obtaining a yellow solid in 60% yield.

[M+H]⁺=298

### Preparation z

### N-{2-[(Dimethylamino)methylene]-1-oxo-2,3,4,9-tetrahydro-1H-carbazol-6-yl}-3-methylbutanamide (VIIg)

The compound was prepared as described for 2-[(dimethylamino)methylene]-6-nitro-2,3,4,9-tetrahydro-1H-carbazol-1-one, obtaining a brownish solid in 60% yield.

[M+H]⁺=340

### Preparation aa

### N-{2-[(dimethylamino)methylene]-1-oxo-2,3,4,9-tetrahydro-1H-carbazol-6-yl)thiophene-2-carboxamide (VIIh)

The compound was prepared as described for 2-[(dimethylamino)methylene]-6-nitro-2,3,4,9-tetrahydro-1H-carbazol-1-one, obtaining a brownish solid that was used as such in the subsequent reaction.

[M+H]⁺=366

### Preparation ab

### 2-[(dimethylamino)methylene]-N-isobutyl-1-oxo-2,3,4,9-tetrahydro-1H-carbazole-6-carboxamide (VIIi)

The compound was prepared as described for 2-[(dimethylamino)methylene]-6-nitro-2,3,4,9-tetrahydro-1H-carbazol-1-one, obtaining a brownish solid that was used as such in the subsequent reaction.

[M+H]⁺=340

### Preparation ac

### 2-[(Dimethylamino)methylene]-6-[(4-methylpiperazin-1-yl)carbonyl]-2,3,4,9-tetrahydro-1H-carbazol-1-one (VIIj)

The compound was prepared as described for 2-[(dimethylamino)methylene]-6-nitro-2,3,4,9-tetrahydro-1H-carbazol-1-one, obtaining a brownish solid that was used as such in the subsequent reaction.

[M+H]⁺=367

### Preparation ad

### 2-(Hydroxymethylene)-1-oxo-2,3,4,9-tetrahydro-1H-carbazole-6-carboxylic acid methyl ester (VIIl)

To methyl 1-oxo-2,3,4,9-tetrahydro-1H-carbazole-6-carboxylate (1.345 g, 1 mmol), dissolved in anhydrous tetrahydrofuran (50 mL), 60% NaH (1.771 g, 8 mmol) was added and the reaction mixture stirred at room temperature for 15 minutes. Methyl formate (1 mL, 16.6 mmol) was slowly added dropwise and the reaction mixture stirred at room temperature for 1 hr. The reaction mixture was poured onto ice-cooled 2N hydrochloric acid (50 mL) under stirring. The organic solvent was removed under reduced pressure and the dark yellow precipitate was filtered, washed with water, dried and used in the subsequent step.

[M+H]^{+=272.}

### Preparation ae

### 2-(Bis-methylsulfanyl-methylene)-9-(tetrahydro-pyran-2-yl)-2,3,4,9-tetrahydro-carbazol-1-one (VIIm)

A mixture of 9-(tetrahydropyran-2-yl)-2,3,4,9-tetrahydro-carbazol-1-one (554 mg, 1.86 mmol) and potassium terbutoxide (414 mg, 3.70 mmol) was dissolved in anhydrous tetrahydrofuran (15 ml). Dimethyltrithiocarbonate (510 mg, 3.70 mmol) and methyl iodide (340 μl, 775 mg, 5.46 mmol) were then added. After stirring for one hour at room temperature, the mixture was poured into iced water (15 ml) and extracted with ethyl acetate (3×10 ml). The combined organic layer was dried over sodium sulphate and evaporated under vacuum. The oil material so obtained was taken up with petroleum ether to yield a yellow crystalline material.

¹H-NMR (DMSOd₆), diagnostic signals (ppm): 7.80 (d, 1H), 7.65 (d, 1H), 7.32 (dd, 1H), 7.12 (dd, 1H), 6.69 (m, 1H), 4.10 (m, 1H), 3.62 (m, 1H), 2.90-3.40 (m, 4H), 2.39 (s, 3H), 2.37 (s, 3H), 1.49-2.25 (m, 6H)

[M+H]⁺=374

Compounds of General Formula (I)

### Example 1

### 7-Nitro-2,4,5,10-tetrahydropyrazolo[3,4-a]carbazole (Ia)

To 2-[(dimethylamino)methylene]-6-nitro-2,3,4,9-tetrahydro-1H-carbazol-1-one (0.4 g, 1.4 mmol) in dimethylformamide (2 mL) and absolute ethanol (7 mL) was added 98% hydrazine hydrate (2 mL) and the mixture was warmed to 90-95° C. under stirring for 1 hr. The reaction mixture was cooled, the precipitate filtered and washed with ethanol. Obtained an orange solid (0.3 g, 1.18 mmol, 84% yield).

Melting Point (M.p.): >260° C.

¹H-NMR (DMSOd₆), diagnostic signals (ppm): 8.5 (m, 1H), 8.0 (dd, 1H), 7.6 (s, 1H), 7.45 (d, 1H), 3.0 (t, 2H), 2.9 (t, 2H). [M+H]+=255

### Example 2

### 2,4,5,10-Tetrahydropyrazolo[3,4-a]carbazole-7-carboxylic acid (Ib)

The compound was prepared as described above for 7-nitro-2,4,5,10-tetrahydropyrazolo[3,4-a]carbazole. After flash chromatography (eluant: CH₂Cl₂/MeOH 20:1, then CH₂Cl₂/MeOH 10:1) obtained a yellow solid in 25% yield two steps. M.p.: >260° C.

¹H-NMR (DMSOd₆), diagnostic signals (ppm): 11.7 (s, 1H), 8.1 (s, 1H), 7.65 (d,1H), 7.55 (s, 1H), 7.36 (d, 1H), 2.9(t, 2H), 2.6 (t, 2H).

[M+H]⁺=254

### Example 3

### 2,4,5,10-Tetrahydropyrazolo[3,4-a]carbazole-6-carboxylic acid (Ic)

The compound was prepared as described above for 7-nitro-2,4,5,10-tetrahydropyrazolo[3,4-a]carbazole. After flash chromatography (eluant: CH₂Cl₂/MeOH 10:1) obtained a yellow solid in 20% yield two steps.

M.p.: >260° C.

¹H-NMR (DMSOd₆), diagnostic signals (ppm): 11.7 (s, 1H), 8.1 (s, 1H), 7.65 (d,1H), 7.55 (s, 1H), 7.36 (d, 1H), 2.9(t, 2H), 2.6 (t, 2H).

[M+H]⁺=254

### Example 4

### 2,4,5,10-Tetrahydropyrazolo[3,4-a]carbazole-8-carboxylic acid (Id)

The compound was prepared as described above for 2,4,5,10-Tetrahydropyrazolo[3,4-a]carbazole-6-carboxylic acid. After flash chromatography (eluant: CH₂Cl₂/MeOH 10:1) obtained a yellow solid in 35% two step yield.

M.p.: >260° C.

¹H-NMR (DMSOd₆), diagnostic signals (ppm): 11.75 (s, 1H), 7.5 (m, 3H), 7.05 (t,1H), 3.15(t, 2H), 2.75 (t, 2H).

[M+H]⁺=254

### Example 5

### Ethyl 2,4,5,10-tetrahydropyrazolo[3,4-a]carbazole-3-carboxylate (Ie)

The compound was prepared as described above for 7-nitro-2,4,5,10-tetrahydropyrazolo[3,4-a]carbazole. After flash chromatography (eluant: CH₂Cl₂/MeOH 30:1) obtained a whitish solid in 35% yield.

M.p. 224-226° C.

¹H-NMR (DMSOd₆), diagnostic signals (ppm): 13.5 (s, 1H), 11.5 (s, 1H), 7.45 (d, 1H), 7.3 (d,1H), 7.0 (2m, 2H), 4.3 (q, 2H), 3.05(t, 2H), 2.95 (t, 2H), 1.35 (t, 3H).

[M+H]⁺⁼282

### Example 6

### N-(1,4,5,10-tetrahydropyrazolo[3,4-a]carbazol-7-yl)acetemide (If)

The compound was prepared as described above for 7-nitro-2,4,5,10-tetrahydropyrazolo[3,4-a]carbazole with no need for chromatography. It was obtained as an orange solid, 25% yield, M.p. 187-193° C.

¹H-NMR (DMSOd₆), diagnostic signals (ppm): 12.4 (s, 1H), 11.25 (s, 1H), 9.65 (s, 1H), 7.7 (s, 1H), 7.5 (s,1H), 7.15 (q, 2H), 2.8 (m,4H), 2.0 (s, 3H).

[M+H]⁺=267

### Example 7

### 3-Methyl-N-(1,4,5,10-tetrahydropyrazolo[3,4-a]carbazol-7-yl)butanamide (Ig)

The compound was prepared as described above for 7-nitro-2,4,5,10-tetrahydropyrazolo[3,4-a]carbazole. After flash chromatography (eluant: CH₂Cl₂/MeOH 15:1) obtained an orange solid in 17% yield.

M.p. 182-185° C.

¹H-NMR (DMSOd₆), diagnostic signals (ppm): 9.6 (s, 1H), 7.7 (s, 1H), 7.5 (s,1H), 7.3-7.1 (q, 2H), 2.8 (t,2H), 2.5 (t, 2H), 2.2-2.0 (m, 3H), 0.95 (d, 6H).

[M+H]⁺=309

### Example 8

### N-(1,4,5,10-tetrahydropyrazolo[3,4-a]carbazol-7-yl) thiophene-2-carboxamide (Ii)

The compound was prepared as described above for 7-nitro-2,4,5,10-tetrahydropyrazolo[3,4-a]carbazole. After flash chromatography (eluant: CH₂Cl₂/MeOH 20:1) obtained a yellow solid in 20% two step yield.

M.p. 176-180° C.

¹H-NMR (DMSOd₆), diagnostic signals (ppm): 12.5 (s, 1H), 11.3 (s, 1H), 10.0 (s, 1H), 8.0 (m, 1H), 7.8 (m,2H), 7.5 (s, 1H), 7.3 (m, 2H), 7.2 (m, 1H), 2.85 (m, 4H).

[M+H]⁺=335

### Example 9

### N-isobutyl-1,4,5,10-tetrahydropyrazolo[3,4-a]carbazole-7-carboxamide (Ij)

The compound was prepared as described above for 7-nitro-2,4,5,10-tetrahydropyrazolo[3,4-a]carbazole. The crude product was precipitated in ether, and obtained as yellow solid (0.015 g, 0.05 mmol, 23% two step yield).

M.p.: 186-190° C.

¹H-NMR (DMSOd₆), diagnostic signals (ppm): 12.5 (s, 1H), 11.6 (s, 1H), 8.25 (t, 1H), 8.05 (s, 1H), 7.6 (m,2H), 7.3 (d, 1H), 3.1 (t,2H), 2.9 (t, 2H), 2.85(t, 2H), 1.85 (m, 1H), 0.9 (d, 6H).

[M+H]⁺=309

### Example 10

### 7-[(4-Methylpiperazin-1-yl)carbonyl]-1,4,5,10-tetrahydropyrazolo[3,4-a]carbazole (Ik)

The compound was prepared as described above for 7-nitro-2,4,5,10-tetrahydropyrazolo[3,4-a]carbazole. The crude product was purified by flash chromatography (eluant: CH₂Cl₂/MeOH 15:1). A whitish solid was obtained in 36% yield.

M.p. 198-205° C.

¹H-NMR (DMSOd₆), diagnostic signals (ppm): 12.5 (s, 1H), 11.6 (s, 1H), 7.5 (d, 2H), 7.3 (d,1H), 7.05 (dd, 1H), 3.5 (bs, 4H), 2.9 (m, 2H), 2.8 (m, 2H), 2.3 (bs, 4H), 2.2 (s, 3H).

[M+H]⁺=336

### Example 11

### Ethyl 1,4,5,10-tetrahydropyrazolo[3,4-a]carbazole-7-carboxylate (Il)

A solution of 1,4,5,10-tetrahydropyrazolo[3,4-a]carbazole-7-carboxylic acid (0.28 g, 1 mmol) in absolute ethanol (30 mL) and two drops of sulphuric acid was refluxed for 10 hrs. After cooling the reaction mixture was cautiously poured into stirred 5% aqueous sodium hydrogencarbonate solution. The precipitate was collected by filtration, washed thoroughly with water and dried. After flash chromatography (eluant: CH₂Cl₂/MeOH 10:1), the title compound was obtained as a yellowish solid (0.23 g, 0.82 mmol, 82% yield).

M.p. 214-216° C.

¹H-NMR (DMSOd₆), diagnostic signals (ppm): 12.5 (a, 1H), 11.7 (s, 1H), 8.3 (s, 1H), 7.7-7.3 (m,3H), 4.6 (q, 2H), 2.8 (t,2H), 2.7 (t, 2H), 1.3 (t, 3H).

[M+H]⁺=282

### Example 12

### Methyl 1,4,5,10-tetrahydropyrazolo[3,4-a]carbazole-8-carboxylate (Im)

The product was prepared analogously to ethyl 2,4,5,10-tetrahydropyrazolo[3,4-a]carbazole-7-carboxylate. Obtained a yellowish solid in 75% yield.

M.p.: 240-243° C.

¹H-NMR (DMSOd₆), diagnostic signals (ppm): 12.65 (s, 1H), 11.85 (s, 1H), 8.0 (s, 1H), 7.7-7.5 (m,3H), 3.8 (s,3H), 2.9 (t, 2H), 2.8 (t, 2H).

[M+H]⁺=268

### Example 13

### 1,4,5,10-Tetrahydropyrazolo[3,4-a]carbazol-7-amine hydrochloride (In)

A mixture of 7-nitro-2,4,5,10-tetrahydropyrazolo[3,4-a]carbazole (see example 1, 0.1 g, 0.39 mmol), powdered iron (0.07 g, 1.2 mmol) and ammonium chloride (0-11 g, 2 mmol) in, methanol (3 mL) and water (1 mL) was refluxed under stirring. After 11 hrs the hot mixture was filtered through a pad of dicalite, the filtrate concentrated and charged on flash silica gel (eluant: CH₂Cl₂/MeOH 10:1). The fractions containing the desired compound were pooled, acidified with HCl in methanol (Congo red) and precipitated in ether. Obtained a tan solid (0.06 g, 0.24 mmol, 60% yield).

M.p. 237-241° C.

¹H-NMR (DMSOd₆), diagnostic signals (ppm): 11.7 (s, 1H), 9.9 (s, 2H), 7.55 (s, 1H), 7.4 (d,2H), 7.0 (d, 1H), 2.9-2.5 (2t, 4H).

[M+H]⁺=225

### Example 14

### 1,4,5,10-Tetrahydropyrazolo[3,4-a]carbazole-3-carboxylic acid hydrochloride (Io)

A solution of ethyl 2,4,5,10-tetrahydropyrazolo[3,4-a]carbazole-3-carboxylate (see example 5, 0.2 g, 0.71 mmol) in tetrahydrofuran (7 mL), ethanol (2 mL) and 2N NaOH (1 mL) was stirred at 75-80° C. for 3 hrs. After cooling the reaction mixture was concentrated, 2N NaOH solution was added and the basic aqueous phase was extracted with dichloromethane. After cooling to 0° C. it was acidified with 2N HCl and extracted with ethyl acetate. After drying over sodium sulphate and concentration under reduced pressure, a pale yellow solid was obtained (0.11 g, 0.44 mmol, 63% yield).

M.p. >260° C.

¹H-NMR (DMSOd₆), diagnostic signals (ppm): 11.4 (s, 1H), 7.5-6.9(m, 4H), 3.05 (m,2H), 2.9 (m, 2H).

[M+H]⁺=254

### Example 15

### 3-methylsulfanyl-1,4,5,10-Tetrahydropyrazolo[3,4-a]carbazole

2-(bis-methylsulfanyl-methylene)-9-(tetrahydro-pyran-2-yl)-2,3,4,9-tetrahydro-carbazol-1-one (50 mg, 0.134 mmol) was dissolved in 1,4-dioxane (1.5 ml). To this solution hydrazine hydrate (50 μl, excess) was added, and the mixture was refluxed overnight. The solvent was evaporated and the crude dissolved in 2 ml dichloromethane. To this solution, trifluoroacetic acid (o.5 ml) was added, and stirring at room temperature was continued for 15 minutes. The solvent was then evaporated to dryness, and the crude purified by silica gel chromatography, eluting with hexane-ethyl acetate 85-15. The solid material obtained was taken up with ethyl ether and the solution filtered off. 20 mg (58% yield) of colorless solid was so isolated.

¹H-NMR (DMSOd₆), diagnostic signals (ppm): 2.45 (s, 3H), 2.75-302 (m, 4H), 6.99 (dt, 1H), 7.06 (dt, 1H), 7.34 (d, 1H), 7.46 (d, 1H), 11.36 (s, 1H), 12.58 (br. S, 1H).

[M+H]⁺=256

Solid Phase Syntheses

### 1-resin-trityl-1,4,5,10-tetrahydropyrazolo[3,4-a]carbazole-7-carboxylate methyl ester

To commercially available chlorotrityl resin (1 eq., declared loading 1.35 mmol/g), a solution of methyl 2,4,5,10-tetrahydropyrazolo[3,4-a]carbazole-7-carboxylate (54 mg, 1.5 eq, 0.2 mmol) and diisopropylethyl amine (0.07 ml, 3 eq, 0.41 mmol) in DCM/dimethylformamde 10:1 (3 ml) was added. The final suspension was shaken for 3 hours and then the resin was filtered, washed with N,N-dimethylformamide, dichloromethane, methanol, dichloromethane (3 times) and dried under positive pressure of nitrogen. The loading was verified by micro cleavage of a resin sample.

### 1-resin-trityl-1,4,5,10-tetrahydropyrazolo[3,4-a]carbazole-7-carboxylic acid

A solution of LiOH*H₂O (42 mg, 5 eq, 1.0 mmol) in tetrahydrofuran/MeOH/H2O 8:1:1 (3 ml) was added to 1-resin-trityl-2,4,5,10-tetrahydropyrazolo[3,4-a]carbazole-7-carboxylate methyl ester (1 eq, 0.2 mmol), and the suspension was shaken for 48 hours at 60° C. The resin was filtered, washed with N,N-dimethylformamide, dichloromethane, methanol, dichloromethane (3 times) and dried under positive pressure of nitrogen. Hydrolysis was verified by means of a micro cleavage of a resin sample showing that the ester was no longer present.

### 1-resin-trityl-N-(furan-2-ylmethyl)-1,4,5,10-tetrahydropyrazolo[3,4-a]carbazole-7-carboxamide

A solution of PyBOP (520 mg, 5 eq, 1.0 mmol) and diisopropylethyl amine (0.171 ml, 5 eq, 1.0 mmol) in dimethylformamide (3 ml) was added to 1-resin-trityl-1,4,5,10-tetrahydropyrazolo[3,4-a]carbazole-7-carboxylic acid (0.2 mmol, 1 eq), and the resulting suspension was shaken. After 30 minutes furan-2-yl-methylamine was added (5 eq, 1.0 mmol), and shaking continued for 24 hours. The resin was washed and a second cycle with the same amount of reagents was performed. The resin was filtered, washed with N,N-dimethylformamide, dichloromethane, methanol, dichloromethane (three times).

### Example 16

### N-(furan-2-ylmethyl)-1,4,5,10-tetrahydropyrazolo[3,4-a]carbazole-7-carboxamide

1′-resin-trityl-N-(furan-2-ylmethyl)-1,4,5,10-tetrahydropyrazolo[3,4-a]carbazole-7-carboxamide was treated with a solution of TFA 5% in DCM the resulting suspension was gently stirred or shaken at 22° C. for 20 minutes. The solution was collected, and the resin washed with dichloromethane, methanol, dichloromethane. The collected organic solution was dried under vacuum to give a crude solid, the, as highlighted by MS-HPLC analysis contained the titled compound and the corresponding dehydro-derivative N-(furan-2-ylmethyl)-1,10-dihdropyrazolo[3,4-a]carbazole-7-carboxamide, in a 1 to 1 ratio. The two were separated by preparative HPLC.

N-(furan-2-ylmethyl)-1,4,5,10-tetrahydropyrazolo[3,4-a]carbazole-7-carboxamide. HPLC Rt=4.6 min; [M+H]⁺=333.

¹H-NMR (DMSO-d₆), diagnostic signals (ppm): 2.87 (t, 2H), 2.96 (t, 2H), 4.5 (d, 2H), 6.29 (m, 1H), 6.42 (m, 1H), 7.37 (d, 1H), 7.57 (s, 1H), 7.58 (d, 1H), 7.62 (d, 1H), 8.1 (s, 1H), 8.77 (t, 1H).

N-(furan-2-ylmethyl)-1,10-dihydropyrazolo[3,4-a]carbazole-7-carboxamide HPLC Rt=4.8 min; [M+H]⁺=331.

Repeating the last two steps with the appropriate amine all the following compounds have been prepared

N-(3-dimethylamino)propyl-1,4,5,10-tetrahydropyrazolo[3,4-a]carbazole-7-carboxamide HPLC Rt=2.6 min; [M+H]+=338.

N-[3-(dimethylamino)propyl]-1,10-dihydropyrazolo[3,4-a]carbazole-7-carboxamide HPLC Rt=2.8 min; [M+H]+=336.

N-(5-hydroxy-1H-pyrazol-3-yl)-1,4,5,10-tetrahydropyrazolo[3,4-a]carbazole-7-carboxamide HPLC Rt=3.4 min; [M+H]+=335.

N-(5-hydroxy-1H-pyrazol-3-yl)-1,10-dihydropyrazolo[3,4-a]carbazole-7-carboxamide HPLC Rt=3.5 min; [M+H]+=333.

N-(3-morpholin-4-yl-propyl)-1,4,5,10-tetrahydropyrazolo[3,4-a]carbazole-7-carboxamide HPLC Rt=2.9 min; [M+H]+=380.

¹H-NMR (DMSO-d₆), diagnostic signals (ppm): 1.7 (bm, 2H), 2.52 (dt, 6H), 2.85 (t, 2H), 2.95 (t, 2H), 3.62 (m, 4H), 7.35 (d, 1H), 7.57 (s, 1H), 7.6 (d, 1H), 8.06 (s, 1H), 8.36 (bs, 1H).

N-(3-morpholin-4-yl-propyl)-1,10-dihydropyrazolo[3,4-a]carbazole-7-carboxamide HPLC Rt=3.1 min; [M+H]+=378.

N-(2-phenylamino-ethyl)-1,4,5,10-tetrahydropyrazolo[3,4-a]carbazole-7-carboxamide HPLC Rt=5.2 min; [M+H]+=372.

N-(2-phenylamino-ethyl)-1,10-dihydropyrazolo[3,4-a]carbazole-7-carboxamide HPLC Rt=5.4 min; [M+H]+=370.

N-[2-(1H-imidazol-4-yl)-ethyl]-1,4,5,10tetrahydropyrazolo[3,4-a]carbazole-7-carboxamide HPLC Rt=2.8 min; [M+H]+=347.

¹H-NMR (DMSO-d6), diagnostic signals (ppm): 2.8 (t, 2H), 2.88 (t, 2H), 2.98 (t, 2H), 3.51 (m, 2H), 6.86 (s, 1H), 7.57 (s, 1H), 7.6 (m, 2H), 8.04 (s, 1H), 8.4 (t, 1H).

N-[2-(1H-imidazol-4-yl)-ethyl]-1,10-dihydropyrazolo[3,4-a]carbazole-7-carboxamide HPLC Rt=3. min; [M+H]+=345.

N-(4-hydroxy-butyl)-1,4,5,10-tetrahydropyrazolo[3,4-a]carbazole-7-carboxamide HPLC Rt=3.1 min; [M+H]+=325.

¹H-NMR (DMSO-d₆), diagnostic signals (ppm): 1.37-1.7 (bm, 4H), 2.77-3 (m, 4H), 3.17-3.5 (m, 4H), 7.3 (m, 1H), 7.53 (s, 1H), 7.56 (dd, 1H), 8 (bs, 1H), 8.25 (bt, 1H).

N-(4-hydroxy-butyl)-1,10-dihydropyrazolo[3,4-a]carbazole-7-carboxamide HPLC Rt=3.3 min; [M+H]+=323.

¹H-NMR (DMSO-d₆), diagnostic signals (ppm): 1.45-1.65 (bm, 4H), 3.23-3.51 (m, 4H), 7.3 (m, 1H), 7.66 (bs, 1H), 7.84 (m, 1H), 8.2 (bs, 1H), 8.35 (bt, 1H).

N-(2-hydroxymethyl-phenyl)-1,4,5,10-tetrahydropyrazolo[3,4-a]carbazole-7-carboxamide HPLC Rt=4.0 min; [M+H]+=307.

N-(2-hydroxymethyl-phenyl)-1,10-dihydropyrazolo[3,4-a]carbazole-7-carboxamide HPLC Rt=4.1 min; [M+H]+=305.

N-(pyridin-4-ylmethyl)-1,4,5,10-tetrahydropyrazolo[3,4-a]carbazole-7-carboxamide HPLC Rt=3.6 min; [M+H]+=344.

¹H-NMR (DMSO-d6), diagnostic signals (ppm): 2.9 (m, 4H), 4.54 (d, 2H), 7.33 (d, 2H), 7.38 (d, 1H), 7.57 (s, 1H), 7.66 (d, 1H), 8.13 (bs, 1H), 8.50 (d, 2H), 8.95 (bt, 1H).

N-(pyridin-4-ylmethyl)-1,10-dihydropyrazolo[3,4-a]carbazole-7-carboxamide HPLC Rt=3.8 min; [M+H]+=342.

N-[(methoxycarbonyl)methyl]-1,4,5,10-tetrahydropyrazolo[3,4-a]carbazole-7-carboxamide HPLC Rt=3.6 min; [M+H]+=325.

N-[(methoxycarbonyl)methyl]-1,10-dihydropyrazolo[3,4-a]carbazole-7-carboxamide HPLC Rt=3.8 min; [M+H]+=323.

[4-(2-ethoxyphenyl)piperazin-1-yl]-1,4,5,10-tetrahydropyrazolo[3,4-a]carbazole-7-carboxamide HPLC Rt=5.6 min; [M+H]+=442.

¹H-NMR (DMSO-d₆), diagnostic signals (ppm): 1.36 (t, 3H), 2.8 (t, 2H), 2.9 (t, 2H), 3 (m, 4H), 3.7 (m, 4H), 4.05 (m, 2H), 6.95 (m, 4H), 7.15 (d, 1H), 7.37 (d, 1H), 7.57 (m, 2H).

[4-(2-ethoxyphenyl)piperazin-1-yl]-1,10-dihydropyrazolo[3,4-a]carbazole-7-carboxamide HPLC Rt=5.8 min; [M+H]+=440.

(4-benzylpiperazin-1-yl)-1,4,5,10-tetrahydropyrazolo[3,4-a]carbazole-7-carboxamide HPLC Rt=4.8 min; [M+H]+=412.

¹H-NMR (DMSO-d₆), diagnostic signals (ppm): 2.4 (m, 4H), 2.8 (t, 2H), 2.9 (t, 2H), 3.7 (m, 6H), 7.09 (d, 1H), 7.35 (m, 6H), 7.57 (m, 2H).

(4-benzylpiperazin-1-yl)-1,10-dihydropyrazolo[3,4-a]carbazole-7-carboxamide HPLC Rt=4.9 min; [M+H]+=410.

[4-phenyl-1-piperidin-4-yl]ethanone-1,4,5,10-tetrahydropyrazolo[3,4-a]carbazole-7-carboxamide HPLC Rt=5.3 min; [M+H]+=439.

¹H-NMR (DMSO-d₆), diagnostic signals (ppm): 1.94 (s, 3H), 2.2 (m, 2H), 2.4 (m, 2H), 2.8 (t, 2H), 2.9 (t, 2H), 3.8 (m, 4H), 7.11 (d, 1H), 7.39 (m, 6H), 7.54 (m, 2H).

[4-phenyl-1-piperidin-4-yl]ethanone-1,10-tetrahydropyrazolo[3,4-a]carbazole-7-carboxamide HPLC Rt=5.4 min; [M+H]+=437

N-methyl-N-(1-naphthylmethyl)-1,4,5,10-tetrahydropyrazolo[3,4-a]carbazole-7-carboxamide HPLC Rt=5.9 min; [M+H]+=407

¹H-NMR (DMSO-d₆), diagnostic signals (ppm): 2.7 (m, 3H), 2.8 (t, 2H), 2.9 (t, 2H), 5.16 (s, 2H), 7.16 (d, 1H), 7.33 (d, 1H), 7.46-7.66 (m, 6H), 7.91 (m, 2H), 8 (m, 2H).

N-methyl-N-(1-naphthylmethyl)-1,10-tetrahydropyrazolo[3,4-a]carbazole-7-carboxamide HPLC Rt=6.0 min; [M+H]+=405

N-ethyl-1,4,5,10-tetrahydropyrazolo[3,4-a]carbazole-7-carboxamide HPLC Rt=4.2 min; [M+H]+=281

¹H-NMR (DMSO-d₆), diagnostic signals (ppm): 1.16 (t, 3H), 2.87 (t, 2H), 2.97 (t, 2H), 3.3 (m, 2H), 7.35 (d, 1H), 7.57 (s, 1H), 7.58 (d, 2H), 8.04 (s, 1H), 8.28 (t, 1H).

N-ethyl-1,4,5,10-tetrahydropyrazolo[3,4-a]carbazole-7-carboxamide HPLC Rt=4.4 min; [M+H]+=479

N-(1-ethylpiperidin-3-yl)-1,4,5,10-tetrahydropyrazolo[3,4-a]carbazole-7-carboxamide HPLC Rt=5.5 min; [M+H]+=364

¹H-NMR (DMSO-d₆), diagnostic signals (ppm): 1.26 (t, 3H), 2 (m, 4H), 2.7-3.7 (m, 10H), 7.39 (d, 1H), 7.58 (s, 1H), 7.60 (d, 1H), 8.06 (s, 1H), 8.39 (d, 1H).

N-(1-ethylpiperidin-3-yl)-1,4,5,10-tetrahydropyrazolo[3,4-a]carbazole-7-carboxamide HPLC Rt=5.7 min; [M+H]+=362

N-neopentyl-1,4,5,10-tetrahydropyrazolo[3,4-a]carbazole-7-carboxamide HPLC Rt=5.0 min; [M+H]+=323

¹H-NMR (DMSO-d₆), diagnostic signals (ppm): 0.93 (s, 9H), 2.87 (m, 2H), 2.98 (m, 2H), 3.15 (d, 2H), 7.36 (d, 1H), 7.57 (s, 1H), 7.60 (d, 1H), 8.06 (s, 1H), 8.15 (t, 1H).

N-neopentyl-1,10-dihydropyrazolo[3,4-a]carbazole-7-carboxamide HPLC Rt=5.2 min; [M+H]+=321

N-(4-methoxy-2-methylphenyl)-1,4,5,10-tetrahydropyrazolo[3,4-a]carbazole-7-carboxamide HPLC Rt=5.0 min; [M+H]+=373

¹H-NMR (DMSO-d₆), diagnostic signals (ppm): 2.23 (s, 3H), 2.88 (m, 2H), 2.9 (m, 2H), 3.77 (s, 3H), 6.81 (m, 1H), 6.86 (d, 1H), 7.25 (d, 1H), 7.42 (d, 1H), 7.58 (s, 1H), 7.70 (d, 1H), 8.20 (s, 1H), 9.61 (s, 1H).

N-(4-methoxy-2-methylphenyl)-1,10-dihydropyrazolo[3,4-a]carbazole-7-carboxamide HPLC Rt=5.2 min; [M+H]+=371

N-[3-(dimethylamino)propyl]-1,4,5,10-tetrahydropyrazolo[3,4-a]carbazole-7-carboxamide HPLC Rt=2.2 min; [M+H]+=338

¹H-NMR (DMSO-d₆), diagnostic signals (ppm): 1.91 (m, 2H), 2.8-3,5(m, 14H), 7.38 (d, 1H), 7.58 (s, 1H), 7.59 (d, 1H), 8.05 (s, 1H), 8.48 (t, 1H).

N-[3-(dimethylamino)propyl]-1,10-dihydropyrazolo[3,4-a]carbazole-7-carboxamide HPLC Rt=2.3 min; [M+H]+=336

N-(2,5-difluorobenzyl)-1,4,5,10-tetrahydropyrazolo[3,4-a]carbazole-7-carboxamide HPLC Rt=5.3 min; [M+H]+=379

¹H-NMR (DMSO-d₆), diagnostic signals (ppm): 2.23 (s, 3H), 2.88 (m, 2H), 2.9 (m, 2H), 3.77 (s, 3H), 6.81 (m, 1H), 6.86 (d, 1H), 7.25 (d, 1H), 7.42 (d, 1H), 7.58 (s, 1H), 7.70 (d, 1H), 8.20 (s, 1H), 9.61 (s, 1H).

N-(2,5-difluorobenzyl)-1,10-dihydropyrazolo[3,4-a]carbazole-7-carboxamide HPLC Rt=5.4 min; [M+H]+=377

N-(2-fluorobenzyl)-1,4,5,10-tetrahydropyrazolo[3,4-a]carbazole-7-carboxamide HPLC Rt=5.0 min; [M+H]+=361

¹H-NMR (DMSO-d₆), diagnostic signals (ppm): 2.88 (m, 2H), 2.9 (m, 2H), 4.55 (m, 2H), 7.15-7.35 (m, 5H), 7.58 (s, 1H), 7.64 (d, 1H), 8.5 (t, 1H).

N-(2-fluorobenzyl)-1,10-dihydropyrazolo[3,4-a]carbazole-7-carboxamide HPLC Rt=5.2 min; [M+H]+=359

N-cyclopentyl-1,4,5,10-tetrahydropyrazolo[3,4-a]carbazole-7-carboxamide HPLC Rt=4.75 min; [M+H]+=321

¹H-NMR (DMSO-d₆), diagnostic signals (ppm): 1.5-2.0 (m, 8H), 2.87 (m, 2H), 2.98 (m, 2H), 4.26 (m, 1H), 7.35 (d, 1H), 7.57 (s, 1H), 7.58 (d, 1H), 8.04 (s, 1H), 8.08 (d, 1H).

N-cyclopentyl-1,10-dihydropyrazolo[3,4-a]carbazole-7-carboxamide HPLC Rt=4.96 min; [M+H]+=319

N-(2-methoxyethyl)-1,4,5,10-tetrahydropyrazolo[3,4-a]carbazole-7-carboxamide HPLC Rt=4.5 min; [M+H]+=311

¹H-NMR (DMSO-d₆), diagnostic signals (ppm): 2.87 (m, 2H), 2.98 (m, 2H), 3.30 (s, 3H), 3.4-3.5 (m, 4H), 7.35 (d, 1H), 7.57 (s, 1H), 7.58 (d, 1H), 8.04 (s, 1H), 8.32 (t, 1H).

N-(2-methoxyethyl)-1,10-dihydropyrazolo[3,4-a]carbazole-7-carboxamide HPLC Rt=4.7 min; [M+H]+=309

N-(4-fluorobenzyl)-1,4,5,10-tetrahydropyrazolo[3,4-a]carbazole-7-carboxamide HPLC Rt=5.1 min; [M+H]+=361

¹H-NMR (DMSO-d₆), diagnostic signals (ppm): 2.87 (m, 2H), 2.98 (m, 2H), 4.48 (d, 2H), 7.16 (t, 2H), 7.38 (m, 3H), 7.57 (s, 1H), 7.63 (d, 1H), 8.11 (s, 1H), 8.88 (t, 1H).

N-(4-fluorobenzyl)-1,10-dihydropyrazolo[3,4-a]carbazole-7-carboxamide HPLC Rt=5.2 min; [M+H]+=360

N-benzyl-1,4,5,10-tetrahydropyrazolo[3,4-a]carbazole-7-carboxamide HPLC Rt=5.0 min; [M+H]+=343

¹H-NMR (DMSO-d₆), diagnostic signals (ppm): 2.87 (m, 2H), 2.98 (m, 2H), 4.52 (d, 2H), 7.20-7.40 (m, 6H), 7.57 (s, 1H), 7.67 (d, 1H), 8.12 (s, 1H), 8.87 (t, 1H).

N-benzyl-1,10-dihydropyrazolo[3,4-a]carbazole-7-carboxamide HPLC Rt=5.2 min; [M+H]+=341

N-(1,2,3,4-tetrahydronaphthalen-1-yl)-1,4,5,10-tetrahydropyrazolo[3,4-a]carbazole-7-carboxamide HPLC Rt=5.8 min; [M+H]+=383

¹H-NMR (DMSO-d₆), diagnostic signals (ppm): 1.6-2.15 (m, 4H), 2.87 (m, 2H), 2.98 (m, 2H), 5.30 (m, 1H), 7.1-7.27 (m, 4H), 7.37 (d, 1H), 7.57 (s, 1H), 7.68 (d, 1H), 8.16 (s, 1H), 8.56 (m, 1H).

N-(1,2,3,4-tetrahydronaphthalen-1-yl)-1,10-dihydropyrazolo[3,4-a]carbazole-7-carboxamide HPLC Rt=5.9 min; [M+H]+=381

N-(3-methoxypropyl)-1,4,5,10-tetrahydropyrazolo[3,4-a]carbazole-7-carboxamide HPLC Rt=3.57 min; [M+H]+=325

¹H-NMR (DMSO-d₆), diagnostic signals (ppm): 1.79 (m, 2H), 2.87 (m, 2H), 2.98 (m, 2H), 3.28 (s, 3H); 3.3-3.5 (m, 4H), 7.36 (d, 1H), 7.57 (s, 1H), 7.60 (d, 1H), 8.04 (s, 1H), 8.28 (t, 1H).

N-(3-methoxypropyl)-1,10-dihydropyrazolo[3,4-a]carbazole-7-carboxamide HPLC Rt=3.7 min; [M+H]+=323

N-[3-(benzyloxy)phenyl]-1,4,5,10-tetrahydropyrazolo[3,4-a]carbazole-7-carboxamide HPLC Rt=6.6 min; [M+H]+=435

¹H-NMR (DMSO-d₆), diagnostic signals (ppm): 2.98 (m, 2H), 3.01 (m, 2H), 5.12 (s, 2H); 6.77 (m, 1H), 7.24 (t,1H); 7.37 (m, 1H), 7.40-7.52 (m, 6H), 7.58 (d, 1H),7.63 (m, 1H); 7.71 (d, 1H); 8.18 (s, 1H), 10.08 (s, 1H).

N-[3-(benzyloxy)phenyl]-1,4,5,10-tetrahydropyrazolo[3,4-a]carbazole-7-carboxamide HPLC Rt=6.8 min; [M+H]+=433

N-cycloheptyl-1,4,5,10-tetrahydropyrazolo[3,4-a]carbazole-7-carboxamide HPLC Rt=5.49 min; [M+H]+=349

¹H-NMR (DMSO-d₆), diagnostic signals (ppm): 1.3-2.0 (m, 12H); 2.85 (m, 2H), 3.0 (m, 2H), 4.0 (m, 1H); 7.37 (m, 1H), 7.58 (d, 1H), 7.63 (m, 1H); 8.05 (m, 2H).

N-cycloheptyl-1,10-dihydropyrazolo[3,4-a]carbazole-7-carboxamide HPLC Rt=5.75 min; [M+H]+=347

N-prop-2-ynyl-1,4,5,10-tetrahydropyrazolo[3,4-a]carbazole-7-carboxamide HPLC Rt=3.77 min; [M+H]+=291

¹H-NMR (DMSO-d₆), diagnostic signals (ppm): 2.85 (m, 2H), 2.95 (m, 2H), 3.09 (m, 1H); 4.08 (m, 2H); 7.37 (m, 1H), 7.58 (d, 1H), 7.62 (m, 1H); 8.08 (s, 1H).

N-prop-2-ynyl-1,10-dihydropyrazolo[3,4-a]carbazole-7-carboxamide HPLC Rt=3.95 min; [M+H]+=289

Solid Phase Syntheses

### 1-resin-trityl-7-nitro-1,4,5,10-tetrahydropyrazolo[3,4-a]carbazole

To commercially available chlorotrityl resin (1 eq, declared loading 1.35 mmol/g), a solution of 7-nitro-2,4,5,10-tetrahydropyrazolo[3,4-a]carbazole (54 mg, 1.5 eq, 0.2 mmol) and diisopropylethyl amine (0.07 ml, 3 eq, 0.41 mmol) in dimethylformamide (3 ml) was added. The final suspension was shaken for 3 hours and then the resin was filtered, washed with N,N-dimethylformide, dichloromethane, methanol, dichloromethane (3 times) and dried under positive pressure of nitrogen. The loading was verified by micro cleavage of a resin sample.

### 1-resin-trityl-7-nitro-1,4,5,10-tetrahydropyrazolo[3,4-a]carbazole

A solution of SnCl₂*2H₂O 3 ml 2M in dimethylformamide was added to 1-resin-trityl-7-nitro-2,4,5,10-tetrahydropyrazolo[3,4-a]carbazole (1 eq, 0.2 mmol), and the suspension was shaken for 48 hours at room temperature. The resin was filtered, washed with N,N-dimethylformamide, dichloromethane, methanol, dichloromethane (3 times) and dried under positive pressure of nitrogen. Hydrolysis was verified by means of a micro cleavage of a resin sample showing that the nitro group was no longer present.

### 1-resin-trityl-3,4-dimethoxy-N-(1,4,5,10-tetrahydropyrazolo[3,4-a]carbazol-7-yl)benzamide

A solution of PyBOP (223 mg, 3 eq, 0.43 mmol) and diisopropylethyl amine (0.074 ml, 3 eq, 0.43 mmol) in dimethylformamide (3 ml) was added to 3,4-dimethoxybenzoic acid (78 mg, 0.43 mmol, 3 eq), and the resulting suspension was shaken. After 30 minutes 1-resin-trytil-2,4,5,10-tetrahydropyrazolo[3,4-a]carbazol-7-amine was added (1 eq, 0.15 mmol), and shaking continued for 24 hours. The resin was filtered, washed with N,N-dimethylformamide, dichloromethane, methanol, dichloromethane (three times).

### Example 17

3,4-dimethoxy-N-(1,4,5,10-tetrahydropyrazolo[3,4-a]carbazol-7-yl)benzamide was treated with a solution of TFA 5% in DCM the resulting suspension was gently stirred or shaken at 22° C. for 20 minutes. The solution was collected, and the resin washed with dichloromethane, methanol, dichloromethane. The collected organic solution was dried under vacuum to give a crude solid, the, as highlighted by MS-HPLC analysis contained the titled compound and the corresponding dehydro-derivative 3,4-dimethoxy-N-(1,10-dihydropyrazolo[3,4-a]carbazol-7-yl)benzamide, in a 2 to 1 ratio. The two were separated by preparative HPLC.

3,4-dimethoxy-N-(1,4,5,10-tetrahydropyrazolo[3,4-a]carbazol-7-yl)benzamide Rt=4.63 min; [M+H]+=389

¹H-NMR (DMSO-d₆), diagnostic signals (ppm): 2.89 (m, 4H); 3.87 (s, 6H); 7.10 (d, 1H); 7.31 (m, 1H); 7.34 (m, 1H), 7.55 (bs, 1H); 7.60 (s, 1H); 7.65 (m, 1H); 7.86 (bs, 1H); 9.91 (s, 1H); 11.35 (s, 1H); 12.50 (s, 1H).

3,4-dimethoxy-N-(1,10-dihydropyrazolo[3,4-a]carbazol-7-yl)benzamide Rt=4.74 min; [M+H]+=387

Repeating the last two steps with the appropriate amine all the following compounds have been prepared

3-(2-methoxyphenyl)-N-(1,4,5,10-tetrahydropyrazolo[3,4-a]carbazol-7-yl)propanamide Rt 5.3 min; [M+H]+=387

¹H-NMR (DMSO-d₆), diagnostic signals (ppm): 2.55 (m, 2H); 2.8-3.0 (m, 6H); 3.82 (s, 3H); 7.10-7.28 (m, 5H); 7.55 (bs, 1H); 7.60 (s, 1H); 7.77 (m, 1H); 9.95 (s, 1H); 11.28 (s, 1H); 12.50 (s, 1H).

3-(2-methoxyphenyl)-N-(1,10-dihydropyrazolo[3,4-a]carbazol-7-yl)-propanamide Rt=5.5 min; [M+H]+=385

4-(4-methylphenyl)-4-ozo-N-(1,4,5,10-tetrahydropyrazolo[3,4-a]carbazol-7-yl)butanamide Rt=5.31 min; [M+H]+=399

¹H-NMR (DMSO-d₆), diagnostic signals (ppm): 2.40 (s, 3H); 2.71 (t, 2H); 2.84 (m, 4H); 3.33 (m, 2H); 7.17 (d, 1H); 7.23 (m, 1H); 7.37 (d, 2H); 7.53 (bs, 1H); 7.78 (m, 1H); 7.91 (d, 2H); 9.79 (s, 1H); 11.26 (s, 1H); 12.48 (s, 1H).

4-(4-methylphenyl)-4-oxo-N-(1,10-dihydropyrazolo[3,4-a]carbazol-7-yl)butanamide Rt=5.45 min; [M+H]+=397

N-2-((benzylamino)carbonyl]-N˜1˜-(1,4,5,10-tetrahydropyrazolo[3,4-a]carbazol-7-yl)glycinamide Rt=5.6 min; [M+H]+=415

¹H-NMR (DMSO-d₆), diagnostic signals (ppm): 2.86 (m, 4H); 3.88 (d, 2H); 3.88 (d, 2H); 4.25 (d, 1H); 6.72 (bt, 2H); 7.17 (m, 1H); 7.2-7.37 (m, 6H); 7.54 (bs, 1H); 7.75 (s, 1H); 9.71 (s, 1H); 11.32 (s, 1H); 12.48 (s, 1H).

N-2-[(benzylamino)carbonyl]-N˜1˜-(1,10-dihydropyrazolo[3,4-a]carbazol-7-yl)glycinamide Rt=5.8 min; [M+H]+=413

(2E)-3-(1H-imidazol-4-yl)-N-(1,4,5,10-tetrahydropyrazolo[3,4-a]carbazol-7-yl)prop-2-enamide Rt=3.14 min; [M+H]+=345

¹H-NMR (DMSO-d₆), diagnostic signals (ppm): 2.88 (m, 4H); 6.6 (m, 1H); 6.9 (s, 1H); 7.10 (s, 1H); 7.25 (s, 1H); 7.55 (bs, 1H); 7.60 (s, 1H); 7.77 (m, 1H); 9.95 (s, 1H); 11.28 (s, 1H); 12.50 (s, 1H).

(2E)-3-(1-imidazol-4-yl)-N-(1,10-dihydropyrazolo[3,4-a]carbazol-7-yl)prop-2-enamide Rt=3.4 min; [M+H]+=343

2-methyl-N-(1,4,5,10-tetrahydropyrazolo[3,4-a]carbazol-7-yl)nicotinamide Rt=3.5 min; [M+H]+=344

¹H-NMR (DMSO-d₆), diagnostic signals (ppm): 2.60 (s, 3H); 2.86 (m, 4H); 7.30 (s, 2H); 7.35 (m, 1H); 7.55 (3, 1H); 7.86 (d, 1H); 7.93 (s, 1H); 8.85 (m, 1H); 10.22 (s, 1H); 11.38 (s, 1H); 12.50 (s, 1H).

2-methyl-N-(1,10-dihydropyrazolo[3,4-a]carbazol-7-yl)nicotinamide Rt=3.81 min; [M+H]+=342

N-(1,4,5,10-tetrahydropyrazolo[3,4-a]carbazol-7-yl)-2-thien-3-ylacetamide Rt=4.79 min; [M+H]+=349

¹H-NMR (DMSO-d₆), diagnostic signals (ppm): 2.86 (m, 4H); 3.65 (s, 2H); 7.14 (m, 1H); 7.18 (m, 1H); 7.34 (m, 1H); 7.51 (m, 1H); 7.54 (s, 1H); 7.78 (s, 1H); 9.90 (s, 1H); 11.31 (s, 1H); 12.50 (s, 1H).

N-(1,10-dihydropyrazolo[3,4-a]carbazol-7-yl)-2-thien-3-ylacetamide Rt=5.0 min; [M+H]+=347

3-methyl-N-(1,4,5,10-tetrahydropyrazolo[3,4-a]carbazol-7-yl)but-2-enamide Rt=4.7 min; [M+H]+=307

¹H-NMR (DMSO-d₆), diagnostic signals (ppm): 2.18 (s, 6H); 2.84 (m, 4H); 5.89 (s, 1H); 7.18 (d, 1H); 7.22 (m, 1H); 7.54 (s, 1H); 7.85 (s, 1H); 9.60 (s, 1H); 11.28 (s, 1H); 12.48 (s, 1H).

3-methyl-N-(1,10-dihydropyrazolo[3,4-a]carbazol-7-yl)but-2-enamide Rt=4.9 min; [M+H]+=305

5-oxo-N-(1,4,5,10-tetrahydropyrazolo[3,4-a]carbazol-7-yl)hexanamide Rt=3.5 min; [M+H]+=337

¹H-NMR (DMSO-d₆), diagnostic signals (ppm): 1.80 (m, 2H); 2.29 (m, 4H); 2.84 (m, 4H); 7.16 (d, 1H); 7.22 (m, 1H); 7.54 (s, 1H); 7.77 (s, 1H); 9.65 (s, 1H); 11.28 (s, 1H); 12.48 (s, 1H).

5-ozo-N-(1,10-dihydropyrazolo[3,4-a]carbazol-7-yl)hexanamide Rt=3.7 min; [M+H]+=335

2,5-dimethyl-N-(1,4,5,10-tetrahydropyrazolo[3,4-a]carbazol-7-yl)-3-furamide Rt=5.2 min; [M+H]+=347

¹H-NMR (DMSO-d₆), diagnostic signals (ppm): 2.55 (s, 6H); 2.88 (m, 4H); 6.69 (s, 1H); 7.28 (m, 2H); 7.54 (s, 1H); 7.82 (s, 1H); 9.42 (s, 1H); 11.33 (s, 1H); 12.49 (s, 1H).

2,5-dimethyl-N-(1,10-dihydropyrazolo[3,4-a]carbazol-7-yl)-3-furamide Rt=5.5 min; [M+H]+=345

1. A method for treating a disease caused by and/or associated with an altered protein kinase activity which comprises administering to a mammal in need thereof an effective amount of a tetracyclic pyrazole derivative represented by formula (I): 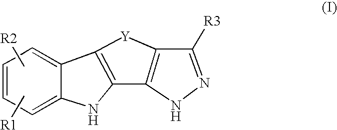
wherein
R1 and R2, being the same or different, are independently hydrogen or halogen atom, nitro, cyano, hydroxy, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl arylaminocarbonyl, heteroarylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl; aryl C₁-C₆ alkyl group, aryl C₁-C₆ alkyloxy group, aryloxy, heteroaryl, heteroaryl C₁-C₆ alkyl group, a straight or branched C₁-C₆ alkylthio, C₁-C₆ alkylsulphinyl, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆ alkylaminosulfonyl and arylaminosulfonyl group;
Y is a —(CH₂)ₙ— group wherein n is 1, 2 or 3, or a carbon-carbon double bond (—CH₂═CH₂—);
R3 is hydrogen atom, cyano, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C8 alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, an aryl, an aryl C₁-C₆ alkyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl C₁-C₆ alkyloxy group, aryloxy, a straight or branched C₁-C₆ alkylthio, arylC₁-C₆ alkylthio, C₁-C₆ alkylsulphinyl group, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl Cₗ-C₆ alkyloxycarbonylamino, aryloxycarbonylamino, an ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆ alkylaminosulfonyl, and arylaminosulfonyl group, or a pharmaceutically acceptable salt thereof.

2. The method of claim 1 wherein the disease caused by and/or associated with an altered protein kinase activity is selected from the group consisting of cancer, cell proliferative disorders, Alzheimer's disease, viral infections, auto-immune diseases and neurodegenerative disorders.

3. The method of claim 2 wherein the cancer is selected from carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderoma pigmentosum, keratoxanthoma, thyroid follicular cancer and Kaposi's sarcoma.

4. The method of claim 2 wherein the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

5. The method of claim 1 which provides tumor angiogenesis and metastasis inhibition.

6. The method of claim 1 further comprising subjecting the mammal in need thereof to a radiation therapy or chemotherapy regimen in combination with at least one cytostatic or cytotoxic agent.

7. The method of claim 1 wherein the mammal in need thereof is a human.

8. A method for inhibiting protein kinase activity which comprises contacting the said kinase with an effective amount of a compound of formula (I) of claim 1.

9. A tetracyclic pyrazole derivative represented by formula (I): 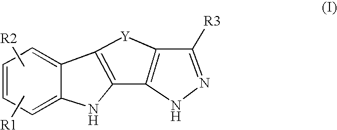
wherein
R1 and R2, being the same or different, are independently hydrogen or halogen atom, nitro, cyano, hydroxy, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl arylaminocarbonyl, heteroarylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl, aryl C₁-C₆ alkyl group, aryl C₁-C₆ alkyloxy group, aryloxy, heteroaryl, heteroaryl C₁-C₆ alkyl group, a straight or branched C₁-C₆ alkylthio, C₁-C₆ alkylsulphinyl, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆ alkylaminosulfonyl and arylaminosulfonyl group;
Y is a —(CH₂)ₙ— group wherein n is 1, 2 or 3, or a carbon-carbon double bond (—CH₂═CH₂—);
R3 is hydrogen atom, cyano, carboxy, hydroxyaminocarbonyl group, or an optionally substituted group selected from aminocarbonyl, amino or sulfonamido group, a straight or branched C₁-C₈ alkyl group, a perfluorinated C₁-C₈ alkyl, a straight or branched C₁-C₈ alkoxy C₁-C₆ alkyl group, a saturated or unsaturated C₃-C₇ cycloalkyl, a saturated or unsaturated C₃-C₇ cycloalkyl C₁-C₆ alkyl, a straight or branched C₂-C₈ alkenyl group, an aryl, an aryl C₁-C₆ alkyl group, a straight or branched C₁-C₈ alkyloxy group, a saturated or unsaturated C₃-C₆ cycloalkyloxy, a straight or branched C₁-C₈ alkyloxy C₁-C₆ alkyloxy group, C₁-C₆ alkyloxycarbonyl, aryloxycarbonyl, aryl C₁-C₆ alkyloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkyloxyaminocarbonyl, aryloxyaminocarbonyl, C₁-C₆ alkylcarbonyloxy, arylcarbonyloxy, C₁-C₆ alkylcarbonyl, arylcarbonyl, heterocyclylcarbonyl, aryl C₁-C₆ alkyloxy group, aryloxy, a straight or branched C₁-C₆ alkylthio, arylC₁-C₆ alkylthio, C₁-C₆ alkylsulphinyl group, C₁-C₆ alkylsulphonyl, arylthio, arylsulphinyl, arylsulphonyl, C₁-C₆ alkylamino, di C₁-C₆ alkylamino, arylamino, aryl C₁-C₆ alkylamino, heteroarylamino, heteroaryl C₁-C₆ alkylamino, C₁-C₆ alkylcarbonylamino, arylcarbonylamino, C₁-C₆ alkyloxycarbonylamino, aryl C₁-C₆ alkyloxycarbonylamino, aryloxycarbonylamino, an ureido, thioureido group, C₁-C₆ alkylaminocarbonylamino, C₁-C₆ dialkylaminocarbonylamino, arylaminocarbonylamino, C₁-C₆ alkylaminothiocarbonylamino, C₁-C₆ dialkylaminothiocarbonylamino, arylaminothiocarbonylamino, C₁-C₆ alkylsulfonylamino, arylsulfonylamino, C₁-C₆ alkylaminosulfonyl, and arylaminosulfonyl group, with the proviso that when R2 and R3 are both hydrogen atoms and Y is a —CH₂—CH₂— group, then R1 is not hydrogen or 7-chloro, 7-bromo atom, 7-cyclohexyl or 7-methyl group, or a pharmaceutically acceptable salt thereof.

10. A 1,4,5,10-tetrahydropyrazolo[3,4-a]carbazole derivative according to claim 9 represented by formula (IA): 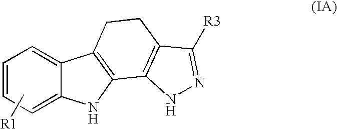
wherein R1 is halogen atom, cyano, nitro, hydroxy, carboxy, aminocarbonyl, hydroxyaminocarbonyl, amino or sulfonamido group, or an optionally substituted group selected from a straight or branched C₁-C₈ alkyl group, a perfluorinated C₁-C₈ alkyl, a saturated or unsaturated C₃-C₇ cycloalkyl, a straight or branched C₁-C₈ alkoxy group, alkyloxycarbonyl, aryloxycarbonyl, heteroaryloxycarbonyl, heteroaryl C₁-C₆ alkyloxycarbonyl, C₁-C₆ alkylaminocarbonyl, C₁-C₆ dialkylaminocarbonyl, arylaminocarbonyl, C₁-C₆ alkoxyaminocarbonyl, aryloxyaminocarbonyl, C₁-C₆ alkylcarbonyloxy, arylcarbonyloxy, an C₁-C₆ alkylamino, arylamino, aryl C₁-C₆ alkylamino, C₁-C₆ alkylcarbonylamino, arylcarbonylamino, aryloxycarbonylamino, C₁-C₆ alkylaminocarbonylamino, C₁-C₆ dialkylaminocarbonylamino, aryl C₁-C₆ alkylaminocarbonylamino, arylaminocarbonylamino, C₁-C₆ alkylsulfonylamino, arylsulfonylamino, C₁-C₆ alkylaminosulfonyl and arylaminosulfonyl;
R3 is hydrogen atom, a carboxy group or an optionally substituted group selected from C₁-C₆ straight or branched alkyl, C₁-C₆ alkyloxycarbonyl, aryl C₁-C₆ alkyloxycarbonyl, C₁-C₆ alkylaminocarbonyl, C₁-C₆ dialkylaminocarbonyl, arylaminocarbonyl and aryl C₁-C₆ alkylaminocarbonyl, with the proviso that when R3 is hydrogen atom, then R1 is not hydrogen or 7-chloro, 7-bromo atom, 7-cyclohexyl or 7-methyl group, or a pharmaceutically acceptable salt thereof.

11. A compound of formula (I), optionally in the form of a pharmaceutically acceptable salt, selected from the group consisting of:
6-fluoro-2,4,5,10-tetrahydropyrazolo[3,4-a]carbazole;
7-fluoro-2,4,5,10-tetrahydropyrazolo[3,4-a]carbazole;
8-fluoro-2,4,5,10-tetrahydropyrazolo[3,4-a]carbazole;
6-chloro-2,4,5,10-tetrahydropyrazolo[3,4-a]carbazole;
8-chloro-2,4,5,10-tetrahydropyrazolo[3,4-a]carbazole;
6-bromo-2,4,5,10-tetrahydropyrazolo[3,4-a]carbazole;
8-bromo-2,4,5,10-tetrahydropyrazolo[3,4-a]carbazole;
6-cyano-2,4,5,10-tetrahydropyrazolo[3,4-a]carbazole;
7-cyano-2,4,5,10-tetrahydropyrazolo[3,4-a]carbazole;
8-cyano-2,4,5,10-tetrahydropyrazolo[3,4-a]carbazole;
6-nitro-2,4,5,10-tetrahydropyrazolo[3,4-a]carbazole;
7-nitro-2,4,5,1-tetrahydropyrazolo[3,4-a]carbazole;
8-nitro-2,4,5,10-tetrahydropyrazolo[3,4-a]carbazole;
6-methyl-2,4,5,10-tetrahydropyrazolo[3,4-a]carbazole;
8-methyl-2,4,5,10-tetrahydropyrazolo[3,4-a]carbazole;
6-trifluoromethyl-2,4,5,10-tetrahydropyrazolo[3,4-a]carbazole;
7-trifluoromethyl-2,4,5,10-tetrahydropyrazolo[3,4-a]carbazole;
8-trifluoromethyl-2,4,5,10-tetrahydropyrazolo[3,4-a]carbazole;
6-methoxy-2,4,5,10-tetrahydropyrazolo[3,4-a]carbazole;
7-methoxy-2,4,5,10-tetrahydropyrazolo[3,4-a]carbazole;
8-methoxy-2,4,5,10-tetrahydropyrazolo[3,4-a]carbazole;
6-hydroxy-2,4,5,10-tetrahydropyrazolo[3,4-a]carbazole;
7-hydroxy-2,4,5,10-tetrahydropyrazolo[3,4-a]carbazole;
8-hydroxy-2,4,5,10-tetrahydropyrazolo[3,4-a]carbazole;
2,4,5,10-tetrahydropyrazolo[3,4-a]carbazole-6-carboxylic acid;
2,4,5,10-tetrahydropyrazolo[3,4-a]carbazole-7-carboxylic acid;
2,4,5,10-tetrahydropyrazolo[3,4-a]carbazole-8-carboxylic acid;
methyl2,4,5,10-tetrahydropyrazolo[3,4-a]carbazole-6-carboxylate;
methyl2,4,5,10-tetrahydropyrazolo[3,4-a]carbazole-7-carboxylate;
methyl2,4,5,10-tetrahydropyrazolo[3,4-a]carbazole-8-carboxylate;
ethyl2,4,5,10-tetrahydropyrazolo[3,4-a]carbazole-6-carboxylate;
ethyl2,4,5,10-tetrahydropyrazolo[3,4-a]carbazole-7-carboxylate;
ethyl2,4,5,10-tetrahydropyrazolo[3,4-a]carbazole-8-carboxylate;
i-butyl2,4,5,10-tetrahydropyrazolo[3,4-a]carbazole-6-carboxylate;
i-butyl2,4,5,10-tetrahydropyrazolo[3,4-a]carbazole-7-carboxylate;
i-butyl2,4,5,10-tetrahydropyrazolo[3,4-a]carbazole-8-carboxylate;
1,4,5,10-tetrahydropyrazolo[3,4-a]carbazole-3-carboxylic acid;
methyl2,4,5,10-tetrahydropyrazolo[3,4-a]carbazole-3-carboxylate;
ethyl2,4,5,10-tetrahydropyrazolo[3,4-a]carbazole-3-carboxylate;
propyl2,4,5,10-tetrahydropyrazolo[3,4-a]carbazole-3-carboxylate;
i-propyl2,4,5,10-tetrahydropyrazolo[3,4-a]carbazole-3-carboxylate;
butyl2,4,5,10-tetrahydropyrazolo[3,4-a]carbazole-3-carboxylate;
i-butyl2,4,5,10-tetrahydropyrazolo[3,4-a]carbazole-3-carboxylate;
1,4,5,10-tetrahydropyrazolo[3,4-a]carbazole-6-carboxamide;
1,4,5,10-tetrahydropyrazolo[3,4-a]carbazole-7-carboxamide;
1,4,5,10-tetrahydropyrazolo[3,4-a]carbazole-8-carboxamide;
N-methyl-1,4,5,10-tetrahydropyrazolo[3,4-a]carbazole-6-carboxamide;
N-methyl-1,4,5,10-tetrahydropyrazolo[3,4-a]carbazole-7-carboxamide;
N-methyl-1,4,5,10-tetrahydropyrazolo[3,4-a]carbazole-8-carboxamide;
N-ethyl-1,4,5,10-tetrahydropyrazolo[3,4-a]carbazole-6-carboxamide;
N-ethyl-1,4,5,10-tetrahydropyrazolo[3,4-a]carbazole-7-carboxamide;
N-ethyl-1,4,5,10-tetrahydropyrazolo[3,4-a]carbazole-8-carboxamide;
N-propyl-1,4,5,10-tetrahydropyrazolo[3,4-a]carbazole-6-carboxamide;
N-propyl-1,4,5,10-tetrahydropyrazolo[3,4-a]carbazole-7-carboxamide;
N-propyl-1,4,5,10-tetrahydropyrazolo[3,4-a]carbazole-8-carboxamide;
N-isopropyl-1,4,5,10-tetrahydropyrazolo[3,4-a]carbazole-6-carboxamide;
N-isopropyl-1,4,5,10-tetrahydropyrazolo[3,4-a]carbazole-7-carboxamide;
N-isopropyl-1,4,5,10-tetrahydropyrazolo[3,4-a]carbazole-8-carboxamide;
N-butyl-1,4,5,10-tetrahydropyrazolo[3,4-a]carbazole-6-carboxamide;
N-butyl-1,4,5,10-tetrahydropyrazolo[3,4-a]carbazole-7-carboxamide;
N-butyl-1,4,5,10-tetrahydropyrazolo[3,4-a]carbazole-8-carboxamide;
N-isobutyl-1,4,5,10-tetrahydropyrazolo[3,4-a]carbazole-6-carboxamide;
N-isobutyl-1,4,5,10-tetrahydropyrazolo[3,4-a]carbazole-7-carboxamide;
N-isobutyl-1,4,5,10-tetrahydropyrazolo[3,4-a]carbazole-8-carboxamide;
N-terbutyl-1,4,5,10-tetrahydropyrazolo[3,4-a]carbazole-6-carboxamide;
N-terbutyl-1,4,5,10-tetrahydropyrazolo[3,4-a]carbazole-7-carboxamide;
N-terbutyl-1,4,5,10-tetrahydropyrazolo[3,4-a]carbazole-8-carboxamide;
N-phenyl-1,4,5,10-tetrahydropyrazolo[3,4-a]carbazole-6-carboxamide;
N-phenyl-1,4,5,10-tetrahydropyrazolo[3,4-a]carbazole-7-carboxamide;
-N-phenyl-1,4,5,10-tetrahydropyrazolo[3,4-a]carbazole-8-carboxamide;
N-benzyl-1,4,5,10-tetrahydropyrazolo[3,4-a]carbazole-6-carboxamide;
N-benzyl-1,4,5,10-tetrahydropyrazolo[3,4-a]carbazole-7-carboxamide;
N-benzyl-1,4,5,10-tetrahydropyrazolo[3,4-a]carbazole-8-carboxamide;
N-(3-dimethylamino)propyl-1,4,5,10-tetrahydropyrazolo[3,4-a]carbazole-6-carboxamide;
N-(3-dimethylamino)propyl-1,4,5,10-tetrahydropyrazolo[3,4-a]carbazole-7-carboxamide;
N-(3-dimethylamino)propyl-1,4,5,10-tetrahydropyrazolo[3,4-a]carbazole-8-carboxamide;
N-(3-dimethylamino)propyl-1,10-dihydropyrazolo[3,4-a]carbazole-6-carboxamide;
N-(3-dimethylamino)propyl-1,10-dihydropyrazolo[3,4-a]carbazole-7-carboxamide;
N-(3-dimethylamino)propyl-1,10-dihydropyrazolo[3,4-a]carbazole-8-carboxamide;
N-(5-hydroxy-1H-pyrazol-3-yl)-1,4,5,10-tetrahydropyrazolo[3,4-a]carbazole-6-carboxamide;
N-(5-hydroxy-1H-pyrazol-3-yl)-1,4,5,10-tetrahydropyrazolo[3,4-a]carbazole-7-carboxamide;
N-(5-hydroxy-1H-pyrazol-3-yl)-1,4,5,10-tetrahydropyrazolo[3,4-a]carbazole-8-carboxamide;
N-(5-hydroxy-1H-pyrazol-3-yl)-1,10-dihydropyrazolo[3,4-a]carbazole-6-carboxamide;
N-(5-hydroxy-1H-pyrazol-3-yl)-1,10-dihydropyrazolo[3,4-a]carbazole-7-carboxamide;
N-(5-hydroxy-1H-pyrazol-3-yl)-1,10-dihydropyrazolo[3,4-a]carbazole-8-carboxamide;
N-(3-morpholin-4-yl-propyl)-1,4,5,10-tetrahydropyrazolo[3,4-a]carbazole-6-carboxamide;
N-(3-morpholin-4-yl-propyl)-1,4,5,10-tetrahydropyrazolo[3,4-a]carbazole-7-carboxamide;
N-(3-morpholin-4-yl-propyl)-1,4,5,10-tetrahydropyrazolo[3,4-a]carbazole-8-carboxamide;
N-(3-morpholin-4-yl-propyl)-1,10-dihydropyrazolo[3,4-a]carbazole-6-carboxamide;
N-(3-morpholin-4-yl-propyl)-1,10-dihydropyrazolo[3,4-a]carbazole-7-carboxamide;
N-(3-morpholin-4-yl-propyl)-1,10-dihydropyrazolo[3,4-a]carbazole-8-carboxamide;
N-(2-phenylamino-ethyl)-1,4,5,10-tetrahydropyrazolo[3,4-a]carbazole-6-carboxamide;
N-(2-phenylamino-ethyl)-1,4,5,10-tetrahydropyrazolo[3,4-a]carbazole-7-carboxamide;
N-(2-phenylamino-ethyl)-1,4,5,10-tetrahydropyrazolo[3,4-a]carbazole-8-carboxamide;
N-(2-phenylamino-ethyl)-1,10-dihydropyrazolo[3,4-a]carbazole-6-carboxamide;
N-(2-phenylamino-ethyl)-1,10-dihydropyrazolo[3,4-a]carbazole-7-carboxamide;
N-(2-phenylamino-ethyl)-1,10-dihydropyrazolo[3,4-a]carbazole-8-carboxamide;
N-[2-(1H-imidazol-4-yl)-ethyl]-1,4,5,10-tetrahydropyrazolo[3,4-a]carbazole-6-carboxamide;
N-[2-(1H-imidazol-4-yl)-ethyl]-1,4,5,10-tetrahydropyrazolo[3,4-a]carbazole-7-carboxamide;
N-[2-(1H-imidazol-4-yl)-ethyl]-1,4,5,10-tetrahydropyrazolo[3,4-a]carbazole-8-carboxamide;
N-[2-(1H-imidazol-4-yl)-ethyl]-1,10-dihydropyrazolo[3,4-a]carbazole-6-carboxamide;
N-[2-(1H-imidazol-4-yl)-ethyl]-1,10-dihydropyrazolo[3,4-a]carbazole-7-carboxamide;
N-[2-(1H-imidazol-4-yl)-ethyl]-1,10-dihydropyrazolo[3,4-a]carbazole-8-carboxamide;
N-(4-hydroxy-butyl)-1,4,5,10-tetrahydropyrazolo[3,4-a]carbazole-6-carboxamide;
N-(4-hydroxy-butyl)-1,4,5,10-tetrahydropyrazolo[3,4-a]carbazole-7-carboxamide;
N-(4-hydroxy-butyl)-1,4,5,10-tetrahydropyrazolo[3,4-a]carbazole-8-carboxamide;
N-(4-hydroxy-butyl)-1,10-dihydropyrazolo[3,4-a]carbazole-6-carboxamide;
N-(4-hydroxy-butyl)-1,10-dihydropyrazolo[3,4-a]carbazole-7-carboxamide;
N-(4-hydroxy-butyl)-1,10-dihydropyrazolo[3,4-a]carbazole-8-carboxamide;
N-(2-hydroxymethyl-phenyl)-1,4,5,10-tetrahydropyrazolo[3,4-a]carbazole-6-carboxamide;
N-(2-hydroxymethyl-phenyl)-1,4,5,10-tetrahydropyrazolo[3,4-a]carbazole-7-carboxamide;
N-(2-hydroxymethyl-phenyl)-1,4,5,10-tetrahydropyrazolo[3,4-a]carbazole-8-carboxamide;
N-(2-hydroxymethyl-phenyl)-1,10-dihydropyrazolo[3,4-a]carbazole-6-carboxamide;
N-(2-hydroxymethyl-phenyl)-1,10-dihydropyrazolo[3,4-a]carbazole-7-carboxamide;
N-(2-hydroxymethyl-phenyl)-1,10-dihydropyrazolo[3,4-a]carbazole-8-carboxamide;
N-(furan-2-ylmethyl)-1,4,5,10-tetrahydropyrazolo[3,4-a]carbazole-6-carboxamide;
N-(furan-2-ylmethyl)-1,4,5,10-tetrahydropyrazolo[3,4-a]carbazole-7-carboxamide;
N-(furan-2-ylmethyl)-1,4,5,10-tetrahydropyrazolo[3,4-a]carbazole-8-carboxamide;
N-(furan-2-ylmethyl)-1,10-dihydropyrazolo[3,4-a]carbazole-6-carboxamide;
N-(furan-2-ylmethyl)-1,10-dihydropyrazolo[3,4-a]carbazole-7-carboxamide;
N-(furan-2-ylmethyl)-1,10-dihydropyrazolo[3,4-a]carbazole-8-carboxamide;
N-(pyridin-4-ylmethyl)-1,4,5,10-tetrahydropyrazolo[3,4-a]carbazole-6-carboxamide;
N-(pyridin-4-ylmethyl)-1,4,5,10-tetrahydropyrazolo[3,4-a]carbazole-7-carboxamide;
N-(pyridin-4-ylmethyl)-1,4,5,10-tetrahydropyrazolo[3,4-a]carbazole-8-carboxamide;
N-(pyridin-4-ylmethyl)-1,10-dihydropyrazolo[3,4-a]carbazole-6-carboxamide;
N-(pyridin-4-ylmethyl)-1,10-dihydropyrazolo[3,4-a]carbazole-7-carboxamide;
N-(pyridin-4-ylmethyl)-1,10-dihydropyrazolo[3,4-a]carbazole-8-carboxamide;
N-[(methoxycarbonyl)methyl]-1,4,5,10-tetrahydropyrazolo[3,4-a]carbazole-6-carboxamide;
N-[(methoxycarbonyl)methyl]-1,4,5,10-tetrahydropyrazolo[3,4-a]carbazole-7-carboxamide;
N-[(methoxycarbonyl)methyl]-1,4,5,10-tetrahydropyrazolo[3,4-a]carbazole-8-carboxamide;
N-[(methoxycarbonyl)methyl]-1,10-dihydropyrazolo[3,4-a]carbazole-6-carboxamide;
N-[(methoxycarbonyl)methyl]-1,10-dihydropyrazolo[3,4-a]carbazole-7-carboxamide;
N-[(methoxycarbonyl)methyl]-1,10-dihydropyrazolo[3,4-a]carbazole-8-carboxamide;
N-(ethane-2-sulfonic acid)-1,4,5,10-tetrahydropyrazolo[3,4-a]carbazole-6-carboxamide;
N-(ethane-2-sulfonic acid)-1,4,5,10-tetrahydropyrazolo[3,4-a]carbazole-7-carboxamide;
N-(ethane-2-sulfonic acid)-1,4,5,10-tetrahydropyrazolo[3,4-a]carbazole-8-carboxamide;
7-[(4-methylpiperazin-1-yl)carbonyl]-1,4,5,10-tetrahydropyrazolo[3,4-a]carbazole;
1,4,5,10-tetrahydropyrazolo[3,4-a]carbazol-6-amine;
1,4,5,10-tetrahydropyrazolo[3,4-a]carbazol-7-amine;
1,4,5,10-tetrahydropyrazolo[3,4-a]carbazol-8-amine;
N-(1,4,5,10-tetrahydropyrazolo[3,4-a]carbazol-6-yl) acetamide;
N-(1,4,5,10-tetrahydropyrazolo[3,4-a]carbazol-7-yl) acetamide;
N-(1,4,5,10-tetrahydropyrazolo[3,4-a]carbazol-8-yl) acetamide;
N-(1,4,5,10-tetrahydropyrazolo[3,4-a]carbazol-6-yl) propanamide;
N-(1,4,5,10-tetrahydropyrazolo[3,4-a]carbazol-7-yl) propanamide;
N-(1,4,5,10-tetrahydropyrazolo[3,4-a]carbazol-8-yl) propanamide;
2-methyl-N-(1,4,5,10-tetrahydropyrazolo[3,4-a]carbazol-6-yl)propanamide;
2-methyl-N-(1,4,5,10-tetrahydropyrazolo[3,4-a]carbazol-7-yl)propanamide;
2-methyl-N-(1,4,5,10-tetrahydropyrazolo[3,4-a]carbazol-8-yl)propanamide;
N-(1,4,5,10-tetrahydropyrazolo[3,4-a]carbazol-6-yl) butanamide;
N-(1,4,5,10-tetrahydropyrazolo[3,4-a]carbazol-7-yl) butanamide;
N-(1,4,5,10-tetrahydropyrazolo[3,4-a]carbazol-8-yl) butanamide;
N-(1,4,5,10-tetrahydropyrazolo[3,4-a]carbazol-6-yl) benzamide;
N-(1,4,5,10-tetrahydropyrazolo[3,4-a]carbazol-7-yl) benzamide;
N-(1,4,5,10-tetrahydropyrazolo[3,4-a]carbazol-8-yl) benzamide;
N-(1,4,5,10-tetrahydropyrazolo[3,4-a]carbazol-6-yl) phenylacetamide;
N-(1,4,5,10-tetrahydropyrazolo[3,4-a]carbazol-7-yl) phenylacetamide;
N-(1,4,5,10-tetrahydropyrazolo[3,4-a]carbazol-8-yl) phenylacetamide;
3-methyl-N-(1,4,5,10-tetrahydropyrazolo[3,4-a]carbazol-6-yl)butanamide;
3-methyl-N-(1,4,5,10-tetrahydropyrazolo[3,4-a]carbazol-7-yl)butanamide;
3-methyl-N-(1,4,5,10-tetrahydropyrazolo[3,4-a]carbazol-8-yl)butanamide;
N-(2,4,5,10-tetrahydropyrazolo[3,4-a]carbazol-7-yl) thiophene-2-carboxamide;
N-methyl-N′-(1,4,5,10-tetrahydropyrazolo[3,4-a]carbazol-7-yl)urea;
N-propyl-N′-(1,4,5,10-tetrahydropyrazolo[3,4-a]carbazol-7-yl)urea;
N-benzyl-N′-(1,4,5,10-tetrahydropyrazolo[3,4-a]carbazol-7-yl)urea;
N-phenyl-N′-(1,4,5,10-tetrahydropyrazolo[3,4-a]carbazol-7-yl)urea;
4,9-dihydro-1H-pyrazolo[4′,3′:4,5]cyclopenta[1,2-b]indole-6-carboxamide;
N-(4,9-dihydro-1H-pyrazolo[4′,3′:4,5]cyclopenta[1,2-b]indol-6-yl)acetamide;
N-(4,9-dihydro-1H-pyrazolo[4′,3′:4,5]cyclopenta[1,2-b]indol-6-yl)-3-methylbutanamide;
N-(4,9-dihydro-1H-pyrazolo[4′,3′:4,5]cyclopenta[1,2-b]indol-6-yl)-2-phenylacetamide;
6-chloro-4,9-dihydro-1H-pyrazolo[4′,3′:4,5]cyclopenta [1,2-b]indole;
N-isobutyl-4,9-dihydro-1H-pyrazolo[4′,3′:4,5]cyclopenta [1,2-b]indole-6-carboxamide;
N-benzyl-4,9-dihydro-1H-pyrazolo[4′,3′:4,5]cyclopenta[1,2-b]indole-6-carboxamide;
ethyl 4,9-dihydro-1H-pyrazolo[4′,3′:4,5]cyclopenta[1,2-b]indole-3-carboxylate;
4,5,6,11-tetrahydro-1H-pyrazolo[4′,3′:6,7]cyclohepta[1,2-b]indole-8-carboxamide;
3-methyl-N-(4,5,6,11-tetrahydro-1H-pyrazolo[4′,3′:6,7]cyclohepta[1,2-b]indol-8-yl)butanamide;
8-chloro-4,5,6,11-tetrahydro-1H-pyrazolo[4′,3′:6,7]cyclohepta[1,2-b]indole;
N-benzyl-4,5,6,11-tetrahydro-1H-pyrazolo[4′,3′:6,7]cyclohepta[1,2-b]indole-8-carboxamide;
N-isobutyl-4,5,6,11-tetrahydro-1H-pyrazolo[4′,3′:6,7]cyclohepta[1,2-b]indole-8-carboxamide;
ethyl4,5,6,11-tetrahydro-1H-pyrazolo[4′,3′:6,7]cyclohepta[1,2-b]indole-3-carboxylate;
N-(4,5,6,11-tetrahydro-1H-pyrazolo[4′,3′:6,7]cyclohepta[1,2-b]indol-8-yl)acetamide;
2-phenyl-N-(4,5,6,11-tetrahydro-1H-pyrazolo[4′,3′:6,7]cyclohepta[1,2-b]indol-8-yl)acetamide and
3-methylsulfanyl-1,4,5,10-tetrahydropyrazolo[3,4-a]carbazole.

12. A process for preparing a compound of formula (I) as defined in claim 9, or a pharmaceutically acceptable salt thereof, which process comprises:
i) treating a compound of formula (VII) 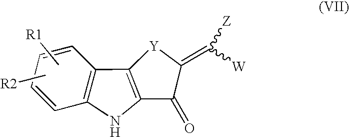
wherein Y is —(CH₂)ₙ—; n, R1 and R2 are as defined in claim 9; W and Z have, respectively, one the following couple of meanings:
e) W is a dialkylamino group, and Z is a hydrogen atom;
f) W is a hydroxy group, and Z is a hydrogen atom, a C₁-C₄ alkoxycarbonyl group or a methyl group;
c) Z is a C₁-C₆ alkylthio or arylC₁-C₆ alkylthio group, and W is:
i) a methylthio group,
ii) a substituted or disubstituted amino group;
iii) a group of general formula —CH(J)(X) where J and X are, the same or different, electron withdrawing groups;
iv) an alkyl or aryl group;
v) an alkyl- or aryl-carbonyl group;
vi) a cyano group or
d) both Z and W are substituted or disubstituted amino groups;
with hydrazine in a suitable solvent to give a compound of general formula (I) wherein Y is a —(CH₂)ₙ— group, n, R1 and R2 are as described above, and R3 is C₁-C₆ alkylthio or arylC₁-C₆ alkylthio group, a substituted or disubstituted amino group; a group of the formula —CH(J)(X) wherein X and J are, the same or different, electron withdrawing groups; a C₁-C₆ alkyl or aryl group; a C₁-C₆ alkyl- or aryl-carbonyl group; a cyano group and
ii) optionally converting a compound of general formula (I) into a different compound of formula (I). if necessary separating a mixture of a compound of formula (I) wherein Y is a —CH₂—CH₂— group and a compound of formula (I) wherein Y is a —CH═CH— group and, if desired, converting a compound of formula (I) into a pharmaceutically acceptable salt thereof or converting a salt into the free compound (I).

13. A process for preparing a compound of formula (I) as defined in claim 9, wherein Y is a carbon-carbon double bond —C═C—, or a pharmaceutically acceptable salt thereof, which process comprises:
i) treating with hydrazine a compound of formula (VIIa) 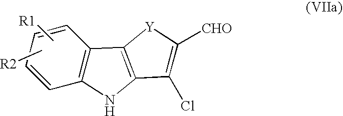
wherein Y is a carbon-carbon double bond —CH═CH—, R1 and R2 are as defined in claim 9, to give a compound of general formula (I) wherein Y is a carbon-carbon double bond and R1, R2 are as described above, and R3 is hydrogen atom, and
ii) optionally converting a compound of general formula (I) into a different compound of formula (I) and, if desired, converting a compound of formula (I) into a pharmaceutically acceptable salt thereof or converting a salt into the free compound (I).

14. A compound of formula VII or VIIa as defined in claims 12 or 13 with the proviso that when R2 is a hydrogen atom, and
i) W is dimethylamino and Z is a hydrogen atom, then R1 is not 7-chloro, hydrogen, 7-bromo atom, 7-cyclohexyl or 7-methyl group, or
ii) W is hydroxy and Z is a hydrogen atom, then R1 is not hydrogen, 7-methoxy group, 7-benzyloxy, or
iii) W is hydroxy and Z is ethyloxycarbonyl group, then R1 is not hydrogen.

15. A process for preparing a compound of the formula (VII) or (VIIa) as defined in claim 14, which process comprises: either i) reacting a compound of formula (VI): 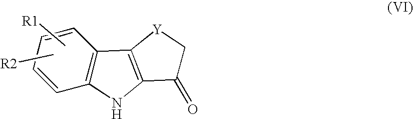
wherein Y, R1 and R2 are as above defined and the indole nitrogen is optionally protected with an appropriate protecting group, with any of the following:
a dialkylacetale of dimethylformammide;
a carboxylic ester;
dimethyl trithiocarbonate and an alkyl iodide or bromide,
to give a compound of general formula (VII) wherein Y is —(CH₂)ₙ—; n, R1 and R2 are as above defined; W and Z have, respectively, one of the following couple of meanings:
a) W is a dialkylamino group, and Z is a hydrogen atom;
b) W is a hydroxy group, and Z is a hydrogen atom, a C₁-C₄ alkoxycarbonyl group or a methyl group;
c) Z is a C₁-C₆ alkylthio or arylC₁-C₆ alkylthio group, and W is a methylthio group;
and iia) optionally reacting a compound of general formula (VII) where R1, R2 and Y are as described above and W and Z are as defined under c) with any of the following:
a′) an aliphatic or aromatic primary or secondary amine;
b′) a compound of general formula W(CH₂)X where W and X are, the same or different, electron withdrawing groups;
c′) an organometallic compound of general formula RM, where R is either an aliphatic or aromatic group, and M represents lithium or magnesium halide;
d′) an organometallic compound of general formula of (CH₃)₂CuLi₂B, where B is a suitable anion species;
e′) an inorganic cyanide;
to give a different compound of general formula (VII) where R1, R2 and Y are as defined above, while Z is a C₁-C₆ alkylthio or arylC₁-C₆ alkylthio group and W is
i) a substituted or disubstituted amino group;
ii) a group of general formula —CH(J) (X) where J and X are, the same or different, electron withdrawing groups;
iii) an alkyl or aryl group;
iv) an alkyl- or aryl-carbonyl group;
v) a cyano group or
d) both Z and W are substituted or disubstituted amino groups,
or ii) reacting another compound of formula (VIa): 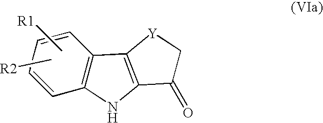
wherein Y is —(CH₂)₂—, R1 and R2 are as above defined, with POCl₃ in dimethylformamide, to give a compound of general formula (VIIa) as defined above.

16. A process according to claims 12 or 13 characterized in that the optional conversion of a compound of formula (I) into a different compound of formula I is carried out by reacting a compound of formula (I) as defined in claim 9 with a suitable activated solid support, then making the desired functionality modifications, and cleaving the resultant compound so as to eliminate the solid support obtaining the desired compound of formula (I).

17. A library of two or more compounds of formula (I) as defined in claim 9, which can be obtained by converting one or more compound of formula (I) supported onto a solid support of the formula (I) as described in claim 16.

18. A pharmaceutical composition comprising an effective amount of pyrazolo[3,4-a]carbazole derivative of formula (I) as defined in claim 9, and at least one pharmaceutically acceptable excipient, carrier or diluent.

19. A pharmaceutical composition according to claim 18 further comprising one or more chemotherapeutic agents, as a combined preparation for simultaneous, separate or sequential use in anticancer therapy.

20. A product or kit comprising a compound of formula (I) as defined in claim 1 or a pharmaceutical composition thereof as defined in claim 18, and one or more chemotherapeutic agents, as a combined preparation for simultaneous, separate or sequential use in anticancer therapy.

21. A compound of formula (I) as defined in claim 1 for use as a medicament.

22. Use of a compound of formula (I) as defined in claim 1 for the preparation of a medicament for the treatment of tumors or cell proliferative disorders.
